(19) United States
(12) Patent Application Publication
(10) Pub. No.: US 2024/0390360 A1
(43) Pub. Date: Nov. 28, 2024

(54) Therapeutic Combinations Of Orally Administered Paclitaxel, A P-gp Inhibitor, And A Checkpoint Inhibitor For The Treatment Of Solid Tumors

(71) Applicant: Athenex, Inc., Buffalo, NY (US)

(72) Inventors: Johnson Yiu-Nam LAU, Houston, TX (US); Min-Fun Rudolf KWAN, Summit, NJ (US); David Lawrence CUTLER, Moorestown, NJ (US); Michael P. SMOLINSKI, Amherst, NY (US)

(21) Appl. No.: 18/689,344

(22) PCT Filed: Sep. 12, 2022
(86) PCT No.: PCT/US2022/076269

§ 371 Date: Mar. 5, 2024

Related U.S. Application Data
(60) Provisional application No. 63/244,673, filed on Sep. 15, 2021.
(60) Provisional application No. 63/242,600, filed on Sep. 10, 2021.

(51) Int. Cl. A61K 31/4725; A61K 9/00; A61K 31/337; A61K 39/00; A61K 39/395; A61P 35/00

(52) U.S. Cl. CPC A61K 31/4725; A61K 9/0053; A61K 31/337; A61K 39/3955; A61P 35/00; A61K 2039/545

(57) ABSTRACT

The application pertains to pharmaceutical combinations of orally administered paclitaxel, a P-gp inhibitor, and a checkpoint inhibitor. The pharmaceutical combinations are suitable for the treatment of a cancer (e.g., lung cancer) in a subject.

### RELATED APPLICATIONS

This application claims priority to, and the benefit of, U.S. Provisional Application No. 63/242,600, filed on Sep. 10, 2021, and U.S. Provisional Application No. 63/244,673, filed on Sep. 15, 2021, the contents of which are incorporated herein by reference in their entirety.

### BACKGROUND

Chemotherapy can kill cancer cells by causing them to elicit an immune response or alternatively, by increasing their susceptibility to an immune attack. In addition, chemotherapy can stimulate anticancer immune effectors either in a direct fashion or by subverting immunosuppressive mechanisms. Encequidar at 15 mg QD plus oral paclitaxel at 205 mg/m² QD×3 days every week has shown to be safe and demonstrated efficacy in treating metastatic breast cancer. Encequidar is a minimally absorbed, oral P-gp inhibitor which prevents intestinal oral paclitaxel efflux. In addition, the combination of IV paclitaxel and pembrolizumab has demonstrated preliminary antitumor activity in two non-small cell lung cancer phase I studies. Accordingly, combining oral paclitaxel and Encequidar (i.e., Compound A) with an immune checkpoint inhibitor may enhance immune activation and tumour cell death.

Current FDA-approved therapies for relapsed/refractory lung cancer patients do not provide a greater than 20% response rate (e.g., monotherapy Keytruda, Opdivo, Taxotere, and Alimta). Thus, there is a need for therapies which utilize the combination of oral paclitaxel and Encequidar with pembrolizumab. The present application addresses the need.

### SUMMARY

The application relates to methods, compositions, medicaments, therapeutic combinations, combinational therapies, and uses for treating a solid tumor in a subject in need thereof.

The application pertains to a combination for use in the treatment of a cancer in a subject in need thereof, wherein the subject is administered paclitaxel orally; Compound A; and a checkpoint inhibitor.

The application pertains to a combination for use in the treatment of a cancer in a subject in need thereof, wherein the subject is administered paclitaxel orally at an amount of about 100 mg to about 400 mg once a day and for 1-7 times a week;

Compound A once a day and for 1-7 times a week; and
a checkpoint inhibitor once a week and for every 1-6 weeks,
wherein Compound A is administered simultaneously with or prior to the paclitaxel.

The application also pertains to a method for treating a cancer in a subject in need thereof, comprising:
a. oral administration of paclitaxel at an amount of about 100 mg to about 400 mg to the subject once a day and for 1-7 times a week;
b. oral administration of Compound A to the subject once a day and for 1-7 times a week; and
c. intravenous administration of a checkpoint inhibitor once a week and for every 1-6 weeks,
wherein Compound A is administered simultaneously with or prior to the paclitaxel.

In some embodiments, the checkpoint inhibitor is pembrolizumab.

The application also pertains to paclitaxel for oral administration for use in combination with Compound A and a checkpoint inhibitor, as described herein, in treating a cancer in a subject in need thereof.

This application also pertains to paclitaxel for oral administration for use with Compound A and a checkpoint inhibitor, as described herein, in a combinational therapy for treating a cancer in a subject in need thereof.

The application also pertains to the use of paclitaxel for oral administration in combination with Compound A and a checkpoint inhibitor, as described herein, in the manufacture of a medicament for treating a cancer in a subject in need thereof.

This application also pertains to Compound A for use in combination with orally administered paclitaxel and a checkpoint inhibitor, as described herein, in treating a cancer in a subject in need thereof.

This application also pertains to Compound A for use with orally administered paclitaxel and a checkpoint inhibitor, as described herein, in a combinational therapy for treating a cancer in a subject in need thereof.

This application also pertains to the use of Compound A in combination with orally administered paclitaxel and a checkpoint inhibitor, as described herein, in the manufacture of a medicament for treating a cancer in a subject in need thereof.

This application also pertains to a checkpoint inhibitor for use in combination with orally administered paclitaxel and Compound A, as described herein, in treating a cancer in a subject in need thereof.

This application also pertains to a checkpoint inhibitor for use with orally administered paclitaxel and Compound A, as described herein, in a combinational therapy for treating a cancer in a subject in need thereof.

This application also pertains to the use of a checkpoint inhibitor in combination with orally administered paclitaxel and Compound A, as described herein, in the manufacture of a medicament for treating a cancer in a subject in need thereof.

This application also pertains to orally administered paclitaxel, Compound A, and a checkpoint inhibitor for use in combination, as described herein, in treating a cancer in a subject in need thereof.

This application also pertains to orally administered paclitaxel, Compound A, and a checkpoint inhibitor for use in a combinational therapy, as described herein, for treating a cancer in a subject in need thereof.

This application also pertains to the use of orally administered paclitaxel, Compound A, and a checkpoint inhibitor in combination, as described herein, in the manufacture of a medicament for treating a cancer in a subject in need thereof.

In some embodiments, the checkpoint inhibitor is pembrolizumab.

### BRIEF DESCRIPTION OF THE DRAWINGS

FIG. 1 is a representative image of the study overview.

FIG. 2 is a graph showing tumour response in evaluable subjects by cohort.

FIG. 3 is a graph showing tumour response in evaluable non-small cell lung cancer subjects.

### DETAILED DESCRIPTION

The application pertains, at least in part, to methods for treating a solid tumor in a subject in need thereof.

In some embodiments, the application pertains to a method for treating a cancer in a subject in need thereof, comprising:
a. oral administration of paclitaxel at an amount of about 100 mg to about 400 mg to the subject once a day and for 1-7 times a week;
b. oral administration of Compound A:

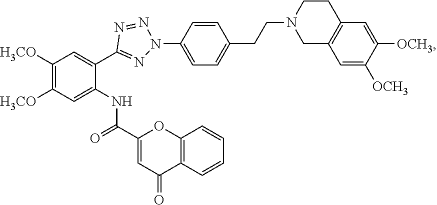 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

to the subject once a day and for 1-7 times a week; and
c. intravenous administration of a checkpoint inhibitor once a week for every 1-6 weeks, wherein Compound A is administered simultaneously with or prior to the paclitaxel.

In some embodiments, the application pertains to a method for treating a cancer in a subject in need thereof, comprising:
a. oral administration of paclitaxel at an amount of about 100 mg to about 400 mg to the subject once a day and for 3 times a week;
b. oral administration of Compound A to the subject once a day and for 3 times a week; and
c. intravenous administration of a checkpoint inhibitor at an amount of about 100 mg to 500 mg once a week on every third or sixth week,
wherein Compound A is administered simultaneously with or prior to the paclitaxel.

In some embodiments, the application pertains to a method for treating a cancer in a subject in need thereof, comprising:
a. oral administration of paclitaxel at an amount of about 270 mg to the subject once a day and for 3 times a week;
b. oral administration of Compound A at an amount of about 15 mg to the subject once a day and for 3 times a week; and
c. intravenous administration of Compound A at an amount of 200 mg to the subject once a week every three weeks,
wherein Compound A is administered simultaneously or prior to the paclitaxel.

In some embodiments, the application pertains to a method for treating a cancer in a subject in need thereof, comprising.
a. oral administration of paclitaxel at an amount of about 270 mg to the subject once a day and for 3 times a week;
b. oral administration of Compound A at an amount of about 15 mg to the subject once a day and for 3 times a week; and
c. intravenous administration of Compound A at an amount of 400 mg to the subject once a week every six weeks,
wherein Compound A is administered simultaneously or prior to the paclitaxel.

The application pertains, at least in part, to Compound A for use in combination with orally administered paclitaxel and a checkpoint inhibitor in treating a cancer in a subject in need thereof.

In some embodiments, the application pertains to Compound A for use in combination with orally administered paclitaxel and a checkpoint inhibitor in treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to the use of Compound A in combination with orally administered paclitaxel and a checkpoint inhibitor in the manufacture of a medicament for the treatment of a cancer in a subject in need thereof.

In some embodiments, the application pertains to the use of Compound A in combination with orally administered paclitaxel and a checkpoint inhibitor, in the manufacture of a medicament for treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to Compound A for use with orally administered paclitaxel and a checkpoint inhibitor in a combinational therapy for treating a cancer in a subject in need thereof.

In some embodiments, the application pertains to Compound A for use with orally administered paclitaxel and a checkpoint inhibitor in a combinational therapy for treating a cancer in a subject in need thereof, wherein Compound A is administered to the subject once a day and for 1-7 times a week;
wherein the subject is also administered paclitaxel orally at an amount of about 100 mg/m² to about 400 mg/m² once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to a medicament for use in a combinational therapy for treating a cancer in a subject in need thereof, wherein the medicament comprises Compound A.

In some embodiments, the application pertains to a medicament for use in a combinational therapy for treating a cancer in a subject in need thereof, wherein the medicament comprises Compound A in combination with orally administered paclitaxel and a checkpoint inhibitor, as described herein, wherein Compound A is administered to the subject once a day and for 1-7 times a week, wherein the subject is administered paclitaxel orally at an amount of about 100 mg/m² to about 400 mg/m² once a day and for 1-7 times a week; wherein the subject is administered a checkpoint inhibitor once a week for every 1-6 weeks, and wherein Compound A is administered simultaneously with or prior to the paclitaxel.

The application pertains, at least in part, to paclitaxel for oral administration for use in combination with Compound A and a checkpoint inhibitor in treating a cancer in a subject in need thereof.

In some embodiments, the application pertains to paclitaxel for oral administration for use in combination with Compound A and a checkpoint inhibitor in treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to the use of paclitaxel for oral administration in combination with Compound A and a checkpoint inhibitor in the manufacture of a medicament for treating a cancer in a subject in need thereof.

In some embodiments, the application pertains to the use of paclitaxel for oral administration in combination with Compound A and a checkpoint inhibitor in the manufacture of a medicament for treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to paclitaxel for oral administration for use with Compound A and a checkpoint inhibitor in a combinational therapy for treating a cancer in a subject in need thereof.

In some embodiments, the application pertains to paclitaxel for oral administration for use with Compound A and a checkpoint inhibitor in a combinational therapy for treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to a medicament for use in a combinational therapy for treating a cancer in a subject in need thereof, wherein the medicament comprises paclitaxel for oral administration.

In some embodiments, the application pertains to a medicament for use in a combinational therapy for treating a cancer in a subject in need thereof, wherein the medicament comprises paclitaxel for oral administration in combination with Compound A and a checkpoint inhibitor, as described herein, wherein paclitaxel is administered to the subject at an amount of about 100 mg/m² to about 400 mg/m² once a day and for 1-7 times a week; wherein the subject is administered Compound A once a day and for 1-7 times a week; wherein the subject is administered a checkpoint inhibitor once a week for every 1-6 weeks, wherein Compound A is administered simultaneously with or prior to the paclitaxel.

The application pertains, at least in part, to a checkpoint inhibitor for use in combination with orally administered paclitaxel and Compound A in the treatment of a cancer in a subject in need thereof.

In some embodiments, the application pertains to a checkpoint inhibitor for use in combination with orally administered paclitaxel and Compound A in the treatment of a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to the use of a checkpoint inhibitor in combination with orally administered paclitaxel and Compound A in the manufacture of a medicament for the treatment of a cancer in a subject in need thereof.

In some embodiments, the application pertains to the use of a checkpoint inhibitor in combination with orally administered paclitaxel and Compound A in the manufacture of a medicament for treating a cancer in a subject in need thereof, wherein the subject is administered paclitaxel orally at an amount of about 100 mg/m² to about 400 mg/m² once a day and for 1-7 times a week;
wherein Compound A is administered to the subject once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to a checkpoint inhibitor for use with orally administered paclitaxel and Compound A in a combinational therapy for treating a cancer in a subject in need thereof.

In some embodiments, the application pertains to a checkpoint inhibitor for use with orally administered paclitaxel and Compound A in a combinational therapy for treating a cancer in a subject in need thereof, wherein Compound A is administered to the subject once a day and for 1-7 times a week:
wherein the subject is also administered paclitaxel orally at an amount of about 100 mg/m² to about 400 mg/m² once a day and for 1-7 times a week; and
wherein a checkpoint inhibitor is administered to a subject once a week for every 1-6 weeks,
wherein Compound A is administered simultaneously with or prior to the paclitaxel.

The application pertains, at least in part, to a medicament for use in a combinational therapy for treating a cancer in a subject in need thereof, wherein the medicament comprises a checkpoint inhibitor.

In some embodiments, the application pertains to a medicament for use in a combinational therapy for treating a cancer in a subject in need thereof, wherein the medicament comprises a checkpoint inhibitor in combination with orally administered paclitaxel and Compound A, as described herein, wherein the checkpoint inhibitor is administered to the subject once a week for every 1-6 weeks, wherein the subject is administered paclitaxel orally at an amount of about 100 mg/m² to about 400 mg/m² once a day and for 1-7 times a week; wherein the subject is administered Compound A once a day and for 1-7 times a week, and wherein Compound A is administered simultaneously with or prior to the paclitaxel.

Unless otherwise indicated, the paclitaxel and Compound A of the present application are administered orally, i.e., through oral administration.

Unless otherwise indicated, the checkpoint inhibitor of the present application is administered intravenously, i.e., through intravenous administration.

For the methods, compositions, medicaments, therapeutic combinations, combinational therapies, and uses described herein (e.g., Compound A for use, use of Compound A in the manufacture of a medicament, Compound A for use with orally administered paclitaxel and a checkpoint inhibitor in a combinational therapy, Compound A for use in combination with orally administered paclitaxel and a checkpoint inhibitor, medicament for use in a combinational therapy, paclitaxel for oral administration for use in combination with Compound A and a checkpoint inhibitor, paclitaxel for oral administration for use with Compound A and a checkpoint inhibitor in a combinational therapy, use of paclitaxel for oral administration in combination with Compound A and a checkpoint inhibitor in the manufacture of a medicament, a checkpoint inhibitor for use, use of a checkpoint inhibitor in the manufacture of a medicament, a checkpoint inhibitor for use with orally administered paclitaxel and Compound A in a combinational therapy, or a checkpoint inhibitor for use in combination with orally administered paclitaxel and Compound A), various embodiments are described below.

### Paclitaxel

In some embodiments, paclitaxel in the present application is formulated for oral administration at the various doses in the methods, compositions, medicaments, therapeutic combinations, combinational therapies, and uses, described herein.

In some embodiments, the paclitaxel is administered at an amount of about 100 mg to about 400 mg.

In some embodiments, the paclitaxel is administered at an amount of about 100 mg to about 350 mg.

In some embodiments, the paclitaxel is administered at an amount of about 100 mg to about 300 mg.

In some embodiments, the paclitaxel is administered at an amount of about 150 mg to about 400 mg.

In some embodiments, the paclitaxel is administered at an amount of about 150 mg to about 390 mg.

In some embodiments, the paclitaxel is administered at an amount of about 150 mg to about 380 mg.

In some embodiments, the paclitaxel is administered at an amount of about 150 mg to about 370 mg.

In some embodiments, the paclitaxel is administered at an amount of about 150 mg to about 360 mg.

In some embodiments, the paclitaxel is administered at an amount of about 150 mg to about 350 mg.

In some embodiments, the paclitaxel is administered at an amount of about 150 mg to about 340 mg.

In some embodiments, the paclitaxel is administered at an amount of about 150 mg to about 330 mg.

In some embodiments, the paclitaxel is administered at an amount of about 150 mg to about 320 mg.

In some embodiments, the paclitaxel is administered at an amount of about 150 mg to about 310 mg.

In some embodiments, the paclitaxel is administered at an amount of about 150 mg to about 300 mg.

In some embodiments, the paclitaxel is administered at an amount of about 150 mg to about 295 mg.

In some embodiments, the paclitaxel is administered at an amount of about 150 mg to about 290 mg.

In some embodiments, the paclitaxel is administered at an amount of about 150 mg to about 285 mg.

In some embodiments, the paclitaxel is administered at an amount of about 150 mg to about 280 mg.

In some embodiments, the paclitaxel is administered at an amount of about 150 mg to about 275 mg.

In some embodiments, the paclitaxel is administered at an amount of about 150 mg to about 270 mg.

In some embodiments, the paclitaxel is administered at an amount of about 150 mg to about 265 mg.

In some embodiments, the paclitaxel is administered at an amount of about 150 mg to about 260 mg.

In some embodiments, the paclitaxel is administered at an amount of about 150 mg to about 255 mg.

In some embodiments, the paclitaxel is administered at an amount of about 150 mg to about 250 mg.

In some embodiments, the paclitaxel is administered at an amount of about 200 mg to about 400 mg.

In some embodiments, the paclitaxel is administered at an amount of about 200 mg to about 390 mg.

In some embodiments, the paclitaxel is administered at an amount of about 200 mg to about 380 mg.

In some embodiments, the paclitaxel is administered at an amount of about 200 mg to about 370 mg.

In some embodiments, the paclitaxel is administered at an amount of about 200 mg to about 360 mg.

In some embodiments, the paclitaxel is administered at an amount of about 200 mg to about 350 mg.

In some embodiments, the paclitaxel is administered at an amount of about 200 mg to about 340 mg.

In some embodiments, the paclitaxel is administered at an amount of about 200 mg to about 330 mg.

In some embodiments, the paclitaxel is administered at an amount of about 200 mg to about 320 mg.

In some embodiments, the paclitaxel is administered at an amount of about 200 mg to about 310 mg.

In some embodiments, the paclitaxel is administered at an amount of about 200 mg to about 300 mg.

In some embodiments, the paclitaxel is administered at an amount of about 200 mg to about 295 mg.

In some embodiments, the paclitaxel is administered at an amount of about 200 mg to about 290 mg.

In some embodiments, the paclitaxel is administered at an amount of about 200 mg to about 285 mg.

In some embodiments, the paclitaxel is administered at an amount of about 200 mg to about 280 mg.

In some embodiments, the paclitaxel is administered at an amount of about 200 mg to about 275 mg.

In some embodiments, the paclitaxel is administered at an amount of about 200 mg to about 270 mg.

In some embodiments, the paclitaxel is administered at an amount of about 200 mg to about 265 mg.

In some embodiments, the paclitaxel is administered at an amount of about 200 mg to about 260 mg.

In some embodiments, the paclitaxel is administered at an amount of about 200 mg to about 255 mg.

In some embodiments, the paclitaxel is administered at an amount of about 200 mg to about 250 mg.

In some embodiments, the paclitaxel is administered at an amount of about 250 mg to about 400 mg.

In some embodiments, the paclitaxel is administered at an amount of about 250 mg to about 390 mg.

In some embodiments, the paclitaxel is administered at an amount of about 250 mg to about 380 mg.

In some embodiments, the paclitaxel is administered at an amount of about 250 mg to about 370 mg.

In some embodiments, the paclitaxel is administered at an amount of about 250 mg to about 360 mg.

In some embodiments, the paclitaxel is administered at an amount of about 250 mg to about 350 mg.

In some embodiments, the paclitaxel is administered at an amount of about 250 mg to about 340 mg.

In some embodiments, the paclitaxel is administered at an amount of about 250 mg to about 330 mg.

In some embodiments, the paclitaxel is administered at an amount of about 250 mg to about 320 mg.

In some embodiments, the paclitaxel is administered at an amount of about 250 mg to about 310 mg.

In some embodiments, the paclitaxel is administered at an amount of about 250 mg to about 300 mg.

In some embodiments, the paclitaxel is administered at an amount of about 250 mg to about 290 mg.

In some embodiments, the paclitaxel is administered at an amount of about 250 mg to about 280 mg.

In some embodiments, the paclitaxel is administered at an amount of about 250 mg to about 270 mg.

In some embodiments, the paclitaxel is administered at an amount of about 250 mg to about 260 mg.

In some embodiments, the paclitaxel is administered at an amount of about 270 mg to about 400 mg.

In some embodiments, the paclitaxel is administered at an amount of about 270 mg to about 390 mg.

In some embodiments, the paclitaxel is administered at an amount of about 270 mg to about 380 mg.

In some embodiments, the paclitaxel is administered at an amount of about 270 mg to about 370 mg.

In some embodiments, the paclitaxel is administered at an amount of about 270 mg to about 360 mg.

In some embodiments, the paclitaxel is administered at an amount of about 270 mg to about 350 mg.

In some embodiments, the paclitaxel is administered at an amount of about 270 mg to about 340 mg.

In some embodiments, the paclitaxel is administered at an amount of about 270 mg to about 330 mg.

In some embodiments, the paclitaxel is administered at an amount of about 270 mg to about 320 mg.

In some embodiments, the paclitaxel is administered at an amount of about 270 mg to about 310 mg.

In some embodiments, the paclitaxel is administered at an amount of about 270 mg to about 300 mg.

In some embodiments, the paclitaxel is administered at an amount of about 270 mg to about 290 mg.

In some embodiments, the paclitaxel is administered at an amount of about 270 mg to about 280 mg.

In some embodiments, the paclitaxel is administered at an amount of about 100 mg.

In some embodiments, the paclitaxel is administered at an amount of about 105 mg.

In some embodiments, the paclitaxel is administered at an amount of about 110 mg.

In some embodiments, the paclitaxel is administered at an amount of about 115 mg.

In some embodiments, the paclitaxel is administered at an amount of about 120 mg.

In some embodiments, the paclitaxel is administered at an amount of about 125 mg.

In some embodiments, the paclitaxel is administered at an amount of about 130 mg.

In some embodiments, the paclitaxel is administered at an amount of about 135 mg.

In some embodiments, the paclitaxel is administered at an amount of about 140 mg.

In some embodiments, the paclitaxel is administered at an amount of about 145 mg.

In some embodiments, the paclitaxel is administered at an amount of about 150 mg.

In some embodiments, the paclitaxel is administered at an amount of about 155 mg.

In some embodiments, the paclitaxel is administered at an amount of about 160 mg.

In some embodiments, the paclitaxel is administered at an amount of about 165 mg.

In some embodiments, the paclitaxel is administered at an amount of about 170 mg.

In some embodiments, the paclitaxel is administered at an amount of about 175 mg.

In some embodiments, the paclitaxel is administered at an amount of about 180 mg.

In some embodiments, the paclitaxel is administered at an amount of about 185 mg.

In some embodiments, the paclitaxel is administered at an amount of about 190 mg.

In some embodiments, the paclitaxel is administered at an amount of about 195 mg.

In some embodiments, the paclitaxel is administered at an amount of about 200 mg.

In some embodiments, the paclitaxel is administered at an amount of about 205 mg.

In some embodiments, the paclitaxel is administered at an amount of about 210 mg.

In some embodiments, the paclitaxel is administered at an amount of about 215 mg.

In some embodiments, the paclitaxel is administered at an amount of about 220 mg.

In some embodiments, the paclitaxel is administered at an amount of about 225 mg.

In some embodiments, the paclitaxel is administered at an amount of about 230 mg.

In some embodiments, the paclitaxel is administered at an amount of about 235 mg.

In some embodiments, the paclitaxel is administered at an amount of about 240 mg.

In some embodiments, the paclitaxel is administered at an amount of about 245 mg.

In some embodiments, the paclitaxel is administered at an amount of about 250 mg.

In some embodiments, the paclitaxel is administered at an amount of about 255 mg.

In some embodiments, the paclitaxel is administered at an amount of about 260 mg.

In some embodiments, the paclitaxel is administered at an amount of about 265 mg.

In some embodiments, the paclitaxel is administered at an amount of about 270 mg.

In some embodiments, the paclitaxel is administered at an amount of about 275 mg.

In some embodiments, the paclitaxel is administered at an amount of about 280 mg.

In some embodiments, the paclitaxel is administered at an amount of about 285 mg.

In some embodiments, the paclitaxel is administered at an amount of about 290 mg.

In some embodiments, the paclitaxel is administered at an amount of about 295 mg.

In some embodiments, the paclitaxel is administered at an amount of about 300 mg.

In some embodiments, the paclitaxel is administered at an amount of about 305 mg.

In some embodiments, the paclitaxel is administered at an amount of about 310 mg.

In some embodiments, the paclitaxel is administered at an amount of about 315 mg.

In some embodiments, the paclitaxel is administered at an amount of about 320 mg.

In some embodiments, the paclitaxel is administered at an amount of about 325 mg.

In some embodiments, the paclitaxel is administered at an amount of about 330 mg.

In some embodiments, the paclitaxel is administered at an amount of about 335 mg.

In some embodiments, the paclitaxel is administered at an amount of about 340 mg.

In some embodiments, the paclitaxel is administered at an amount of about 345 mg.

In some embodiments, the paclitaxel is administered at an amount of about 350 mg.

In some embodiments, the paclitaxel is administered at an amount of about 355 mg.

In some embodiments, the paclitaxel is administered at an amount of about 360 mg.

In some embodiments, the paclitaxel is administered at an amount of about 365 mg.

In some embodiments, the paclitaxel is administered at an amount of about 370 mg.

In some embodiments, the paclitaxel is administered at an amount of about 375 mg.

In some embodiments, the paclitaxel is administered at an amount of about 380 mg.

In some embodiments, the paclitaxel is administered at an amount of about 385 mg.

In some embodiments, the paclitaxel is administered at an amount of about 390 mg.

In some embodiments, the paclitaxel is administered at an amount of about 395 mg.

In some embodiments, the paclitaxel is administered at an amount of about 400 mg.

In some embodiments, the paclitaxel is administered at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, the paclitaxel is administered at an amount of about 250 mg, about 255 mg, about 260 mg, about 265 mg, about 270 mg, about 275 mg, about 280 mg, about 285 mg, about 290 mg, about 295 mg, or about 300 mg.

In some embodiments, paclitaxel in the present application is formulated for oral administration at the various doses described herein with the various dosing frequencies in the methods, compositions, medicaments, therapeutic combinations, combinational therapies, and uses, described herein.

In some embodiments, the paclitaxel is administered 1-7 times per week. In some embodiments, the paclitaxel is administered on consecutive days.

In some embodiments, the paclitaxel is administered 2-7 times per week. In some embodiments, the paclitaxel is administered on consecutive days.

In some embodiments, the paclitaxel is administered 3-7 times per week. In some embodiments, the paclitaxel is administered on consecutive days.

In some embodiments, the paclitaxel is administered 4-7 times per week. In some embodiments, the paclitaxel is administered on consecutive days.

In some embodiments, the paclitaxel is administered 5-7 times per week. In some embodiments, the paclitaxel is administered on consecutive days.

In some embodiments, the paclitaxel is administered 6-7 times per week. In some embodiments, the paclitaxel is administered on consecutive days.

In some embodiments, the paclitaxel is administered 2-6 times per week. In some embodiments, the paclitaxel is administered 2-5 times per week. In some embodiments, the paclitaxel is administered 2-4 times per week. In some embodiments, the paclitaxel is administered 2-3 times per week. In some embodiments, the paclitaxel is administered on consecutive days.

In some embodiments, the paclitaxel is administered 3-6 times per week. In some embodiments, the paclitaxel is administered 3-5 times per week. In some embodiments, the paclitaxel is administered 3-4 times per week. In some embodiments, the paclitaxel is administered on consecutive days.

In some embodiments, the paclitaxel is administered 4-6 times per week. In some embodiments, the paclitaxel is administered 4-5 times per week. In some embodiments, the paclitaxel is administered on consecutive days.

In some embodiments, the paclitaxel is administered 5-6 times per week. In some embodiments, the paclitaxel is administered on consecutive days.

In some embodiments, the paclitaxel is administered at least 1 time per week. In some embodiments, the paclitaxel is administered on consecutive days.

In some embodiments, the paclitaxel is administered at least 2 times per week. In some embodiments, the paclitaxel is administered on consecutive days.

In some embodiments, the paclitaxel is administered at least 3 times per week. In some embodiments, the paclitaxel is administered on consecutive days.

In some embodiments, the paclitaxel is administered at least 4 times per week. In some embodiments, the paclitaxel is administered on consecutive days.

In some embodiments, the paclitaxel is administered at least 5 times per week. In some embodiments, the paclitaxel is administered on consecutive days.

In some embodiments, the paclitaxel is administered at least 6 times per week. In some embodiments, the paclitaxel is administered on consecutive days.

In some embodiments, the paclitaxel is administered daily per week.

In some embodiments, the paclitaxel is administered at least 2 times per week at an amount of about 150 mg to about 400 mg.

In some embodiments, the paclitaxel is administered at least 2 times per week at an amount of about 200 mg to about 400 mg.

In some embodiments, the paclitaxel is administered at least 2 times per week at an amount of about 250 mg to about 350 mg.

In some embodiments, the paclitaxel is administered at least 2 times per week at an amount of about 270 mg to about 330 mg.

In some embodiments, the paclitaxel is administered at least 2 times per week at an amount of about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 2 times per week at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 2 times per week at an amount of about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, the paclitaxel is administered at least 3 times per week at an amount of about 150 mg to about 400 mg.

In some embodiments, the paclitaxel is administered at least 3 times per week at an amount of about 200 mg to about 400 mg.

In some embodiments, the paclitaxel is administered at least 3 times per week at an amount of about 250 mg to about 350 mg.

In some embodiments, the paclitaxel is administered at least 3 times per week at an amount of about 270 mg to about 330 mg.

In some embodiments, the paclitaxel is administered at least 3 times per week at an amount of about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 3 times per week at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 3 times per week at an amount of about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, the paclitaxel is administered at least 4 times per week at an amount of about 150 mg to about 400 mg.

In some embodiments, the paclitaxel is administered at least 4 times per week at an amount of about 200 mg to about 400 mg.

In some embodiments, the paclitaxel is administered at least 4 times per week at an amount of about 250 mg to about 350 mg.

In some embodiments, the paclitaxel is administered at least 4 times per week at an amount of about 270 mg to about 330 mg.

In some embodiments, the paclitaxel is administered at least 4 times per week at an amount of about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 4 times per week at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 4 times per week at an amount of about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, the paclitaxel is administered at least 5 times per week at an amount of about 150 mg to about 400 mg.

In some embodiments, the paclitaxel is administered at least 5 times per week at an amount of about 200 mg to about 400 mg.

In some embodiments, the paclitaxel is administered at least 5 times per week at an amount of about 250 mg to about 350 mg.

In some embodiments, the paclitaxel is administered at least 5 times per week at an amount of about 270 mg to about 330 mg.

In some embodiments, the paclitaxel is administered at least 5 times per week at an amount of about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 5 times per week at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 5 times per week at an amount of about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, the paclitaxel is administered at least 6 times per week at an amount of about 150 mg to about 400 mg.

In some embodiments, the paclitaxel is administered at least 6 times per week at an amount of about 200 mg to about 400 mg.

In some embodiments, the paclitaxel is administered at least 6 times per week at an amount of about 250 mg to about 350 mg.

In some embodiments, the paclitaxel is administered at least 6 times per week at an amount of about 270 mg to about 330 mg.

In some embodiments, the paclitaxel is administered at least 6 times per week at an amount of about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 6 times per week at an amount of about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, or about 400 mg. In some embodiments, the paclitaxel is administered at least 6 times per week at an amount of about 270 mg, about 275 mg, about 280 mg, about 285 mg, about 290 mg, about 295 mg, about 300 mg, about 305 mg, about 310 mg, about 315 mg, about 320 mg, about 325 mg, or about 330 mg.

In some embodiments, paclitaxel is administered for a three week cycle.

In some embodiments, paclitaxel is administered for one week of a three week cycle.

In some embodiments, paclitaxel is administered at least once a week for one week of a three week cycle.

In some embodiments, paclitaxel is administered at least twice a week for one week of a three week cycle.

In some embodiments, paclitaxel is administered at least three times a week for one week of a three week cycle.

In some embodiments, paclitaxel is administered at least four times a week for one week of a three week cycle.

In some embodiments, paclitaxel is administered at least five times a week for one week of a three week cycle.

In some embodiments, paclitaxel is administered at least six times a week for one week of a three week cycle.

In some embodiments, paclitaxel is administered at least daily for one week of a three week cycle.

In some embodiments, paclitaxel is administered at least once a week for one week of a three week cycle from about 100 mg to about 400 mg.

In some embodiments, paclitaxel is administered at least twice a week for one week of a three week cycle from about 100 mg to about 400 mg.

In some embodiments, paclitaxel is administered at least three times a week for one week of a three week cycle from about 100 mg to about 400 mg.

In some embodiments, paclitaxel is administered at least four times a week for one week of a three week cycle from about 100 mg to about 400 mg.

In some embodiments, paclitaxel is administered at least five times a week for one week of a three week cycle from about 100 mg to about 400 mg.

In some embodiments, paclitaxel is administered at least six times a week for one week of a three week cycle from about 100 mg to about 400 mg.

In some embodiments, paclitaxel is administered at least daily for one week of a three week cycle from about 100 mg to about 400 mg.

In some embodiments, paclitaxel is administered at least once a week for one week of a three week cycle from about 250 mg to about 400 mg.

In some embodiments, paclitaxel is administered at least twice a week for one week of a three week cycle from about 250 mg to about 400 mg.

In some embodiments, paclitaxel is administered at least three times a week for one week of a three week cycle from about 250 mg to about 400 mg.

In some embodiments, paclitaxel is administered at least four times a week for one week of a three week cycle from about 250 mg to about 400 mg.

In some embodiments, paclitaxel is administered at least five times a week for one week of a three week cycle from about 250 mg to about 400 mg.

In some embodiments, paclitaxel is administered at least six times a week for one week of a three week cycle from about 250 mg to about 400 mg.

In some embodiments, paclitaxel is administered at least daily for one week of a three week cycle from about 250 mg to about 400 mg.

In some embodiments, paclitaxel is administered at least once a week for one week of a three week cycle at about 270 mg.

In some embodiments, paclitaxel is administered at least twice a week for one week of a three week cycle at about 270 mg.

In some embodiments, paclitaxel is administered at least three times a week for one week of a three week cycle at about 270 mg.

In some embodiments, paclitaxel is administered at least four times a week for one week of a three week cycle at about 270 mg.

In some embodiments, paclitaxel is administered at least five times a week for one week of a three week cycle at about 270 mg.

In some embodiments, paclitaxel is administered at least six times a week for one week of a three week cycle at about 270 mg.

In some embodiments, paclitaxel is administered at least daily for one week of a three week cycle from at about 270 mg.

In some embodiments, paclitaxel is administered at least once a week for one week of a three week cycle at about 300 mg.

In some embodiments, paclitaxel is administered at least twice a week for one week of a three week cycle at about 300 mg.

In some embodiments, paclitaxel is administered at least three times a week for one week of a three week cycle at about 300 mg.

In some embodiments, paclitaxel is administered at least four times a week for one week of a three week cycle at about 300 mg.

In some embodiments, paclitaxel is administered at least five times a week for one week of a three week cycle at about 300 mg.

In some embodiments, paclitaxel is administered at least six times a week for one week of a three week cycle at about 300 mg.

In some embodiments, paclitaxel is administered at least daily for one week of a three week cycle from at about 300 mg.

In some embodiments, paclitaxel is administered at least once a week for one week of a three week cycle at about 330 mg.

In some embodiments, paclitaxel is administered at least twice a week for one week of a three week cycle at about 330 mg.

In some embodiments, paclitaxel is administered at least three times a week for one week of a three week cycle at about 330 mg.

In some embodiments, paclitaxel is administered at least four times a week for one week of a three week cycle at about 330 mg.

In some embodiments, paclitaxel is administered at least five times a week for one week of a three week cycle at about 330 mg.

In some embodiments, paclitaxel is administered at least six times a week for one week of a three week cycle at about 330 mg.

In some embodiments, paclitaxel is administered at least daily for one week of a three week cycle from at about 330 mg.

In some embodiments, paclitaxel is administered for two weeks of a three week cycle.

In some embodiments, paclitaxel is administered at least once a week for two weeks of a three week cycle.

In some embodiments, paclitaxel is administered at least twice a week for two weeks of a three week cycle.

In some embodiments, paclitaxel is administered at least three times a week for two weeks of a three week cycle.

In some embodiments, paclitaxel is administered at least four times a week for two weeks of a three week cycle.

In some embodiments, paclitaxel is administered at least five times a week for two weeks of a three week cycle.

In some embodiments, paclitaxel is administered at least six times a week for two weeks of a three week cycle.

In some embodiments, paclitaxel is administered at least daily for two weeks of a three week cycle.

In some embodiments, paclitaxel is administered at least once a week for two weeks of a three week cycle from about 100 mg to about 400 mg.

In some embodiments, paclitaxel is administered at least twice a week for two weeks of a three week cycle from about 100 mg to about 400 mg.

In some embodiments, paclitaxel is administered at least three times a week for two weeks of a three week cycle from about 100 mg to about 400 mg.

In some embodiments, paclitaxel is administered at least four times a week for two weeks of a three week cycle from about 100 mg to about 400 mg.

In some embodiments, paclitaxel is administered at least five times a week for two weeks of a three week cycle from about 100 mg to about 400 mg.

In some embodiments, paclitaxel is administered at least six times a week for two weeks of a three week cycle from about 100 mg to about 400 mg.

In some embodiments, paclitaxel is administered at least daily for two weeks of a three week cycle from about 100 mg to about 400 mg.

In some embodiments, paclitaxel is administered at least once a week for two weeks of a three week cycle from about 250 mg to about 400 mg.

In some embodiments, paclitaxel is administered at least twice a week for two weeks of a three week cycle from about 250 mg to about 400 mg.

In some embodiments, paclitaxel is administered at least three times a week for two weeks of a three week cycle from about 250 mg to about 400 mg.

In some embodiments, paclitaxel is administered at least four times a week for two weeks of a three week cycle from about 250 mg to about 400 mg.

In some embodiments, paclitaxel is administered at least five times a week for two weeks of a three week cycle from about 250 mg to about 400 mg.

In some embodiments, paclitaxel is administered at least six times a week for two weeks of a three week cycle from about 250 mg to about 400 mg.

In some embodiments, paclitaxel is administered at least daily for two weeks of a three week cycle from about 250 mg to about 400 mg.

In some embodiments, paclitaxel is administered at least once a week for two weeks of a three week cycle at about 270 mg.

In some embodiments, paclitaxel is administered at least twice a week for two weeks of a three week cycle at about 270 mg.

In some embodiments, paclitaxel is administered at least three times a week for two weeks of a three week cycle at about 270 mg.

In some embodiments, paclitaxel is administered at least four times a week for two weeks of a three week cycle at about 270 mg.

In some embodiments, paclitaxel is administered at least five times a week for two weeks of a three week cycle at about 270 mg.

In some embodiments, paclitaxel is administered at least six times a week for two weeks of a three week cycle at about 270 mg.

In some embodiments, paclitaxel is administered at least daily for two weeks of a three week cycle at about 270 mg.

In some embodiments, paclitaxel is administered at least once a week for two weeks of a three week cycle at about 300 mg.

In some embodiments, paclitaxel is administered at least twice a week for two weeks of a three week cycle at about 300 mg.

In some embodiments, paclitaxel is administered at least three times a week for two weeks of a three week cycle at about 300 mg.

In some embodiments, paclitaxel is administered at least four times a week for two weeks of a three week cycle at about 300 mg.

In some embodiments, paclitaxel is administered at least five times a week for two weeks of a three week cycle at about 300 mg.

In some embodiments, paclitaxel is administered at least six times a week for two weeks of a three week cycle at about 300 mg.

In some embodiments, paclitaxel is administered at least daily for two weeks of a three week cycle at about 300 mg.

In some embodiments, paclitaxel is administered at least once a week for two weeks of a three week cycle at about 330 mg.

In some embodiments, paclitaxel is administered at least twice a week for two weeks of a three week cycle at about 330 mg.

In some embodiments, paclitaxel is administered at least three times a week for two weeks of a three week cycle at about 330 mg.

In some embodiments, paclitaxel is administered at least four times a week for two weeks of a three week cycle at about 330 mg.

In some embodiments, paclitaxel is administered at least five times a week for two weeks of a three week cycle at about 330 mg.

In some embodiments, paclitaxel is administered at least six times a week for two weeks of a three week cycle at about 330 mg.

In some embodiments, paclitaxel is administered at least daily for two weeks of a three week cycle at about 330 mg.

In some embodiments, paclitaxel is administered for three weeks of a three week cycle.

In some embodiments, paclitaxel is administered at least once a week for three weeks of a three week cycle.

In some embodiments, paclitaxel is administered at least twice a week for three weeks of a three week cycle.

In some embodiments, paclitaxel is administered at least three times a week for three weeks of a three week cycle.

In some embodiments, paclitaxel is administered at least four times a week for three weeks of a three week cycle.

In some embodiments, paclitaxel is administered at least five times a week for three weeks of a three week cycle.

In some embodiments, paclitaxel is administered at least six times a week for three weeks of a three week cycle.

In some embodiments, paclitaxel is administered at least daily for three weeks of a three week cycle.

In some embodiments, paclitaxel is administered at least once a week for three weeks of a three week cycle from about 100 mg to about 400 mg.

In some embodiments, paclitaxel is administered at least twice a week for three weeks of a three week cycle from about 100 mg to about 400 mg.

In some embodiments, paclitaxel is administered at least three times a week for three weeks of a three week cycle from about 100 mg to about 400 mg.

In some embodiments, paclitaxel is administered at least four times a week for three weeks of a three week cycle from about 100 mg to about 400 mg.

In some embodiments, paclitaxel is administered at least five times a week for three weeks of a three week cycle from about 100 mg to about 400 mg.

In some embodiments, paclitaxel is administered at least six times a week for three weeks of a three week cycle from about 100 mg to about 400 mg.

In some embodiments, paclitaxel is administered at least daily for three weeks of a three week cycle from about 100 mg to about 400 mg.

In some embodiments, paclitaxel is administered at least once a week for three weeks of a three week cycle from about 250 mg to about 400 mg.

In some embodiments, paclitaxel is administered at least twice a week for three weeks of a three week cycle from about 250 mg to about 400 mg.

In some embodiments, paclitaxel is administered at least three times a week for three weeks of a three week cycle from about 250 mg to about 400 mg.

In some embodiments, paclitaxel is administered at least four times a week for three weeks of a three week cycle from about 250 mg to about 400 mg.

In some embodiments, paclitaxel is administered at least five times a week for three weeks of a three week cycle from about 250 mg to about 400 mg.

In some embodiments, paclitaxel is administered at least six times a week for three weeks of a three week cycle from about 250 mg to about 400 mg.

In some embodiments, paclitaxel is administered at least daily for three weeks of a three week cycle from about 250 mg to about 400 mg.

In some embodiments, paclitaxel is administered at least once a week for three weeks of a three week cycle at about 270 mg.

In some embodiments, paclitaxel is administered at least twice a week for three weeks of a three week cycle at about 270 mg.

In some embodiments, paclitaxel is administered at least three times a week for three weeks of a three week cycle at about 270 mg.

In some embodiments, paclitaxel is administered at least four times a week for three weeks of a three week cycle at about 270 mg.

In some embodiments, paclitaxel is administered at least five times a week for three weeks of a three week cycle at about 270 mg.

In some embodiments, paclitaxel is administered at least six times a week for three weeks of a three week cycle at about 270 mg.

In some embodiments, paclitaxel is administered at least daily for three weeks of a three week cycle at about 270 mg.

In some embodiments, paclitaxel is administered at least once a week for three weeks of a three week cycle at about 300 mg.

In some embodiments, paclitaxel is administered at least twice a week for three weeks of a three week cycle at about 300 mg.

In some embodiments, paclitaxel is administered at least three times a week for three weeks of a three week cycle at about 300 mg.

In some embodiments, paclitaxel is administered at least four times a week for three weeks of a three week cycle at about 300 mg.

In some embodiments, paclitaxel is administered at least five times a week for three weeks of a three week cycle at about 300 mg.

In some embodiments, paclitaxel is administered at least six times a week for three weeks of a three week cycle at about 300 mg.

In some embodiments, paclitaxel is administered at least daily for three weeks of a three week cycle at about 300 mg.

In some embodiments, paclitaxel is administered at least once a week for three weeks of a three week cycle at about 330 mg.

In some embodiments, paclitaxel is administered at least twice a week for three weeks of a three week cycle at about 330 mg.

In some embodiments, paclitaxel is administered at least three times a week for three weeks of a three week cycle at about 330 mg.

In some embodiments, paclitaxel is administered at least four times a week for three weeks of a three week cycle at about 330 mg.

In some embodiments, paclitaxel is administered at least five times a week for three weeks of a three week cycle at about 330 mg.

In some embodiments, paclitaxel is administered at least six times a week for three weeks of a three week cycle at about 330 mg.

In some embodiments, paclitaxel is administered at least daily for three weeks of a three week cycle at about 330 mg.

### Compound A

In some embodiments, Compound A or a salt thereof (e.g., methanesulfonate salt) in the present application is formulated for oral administration at the various doses in the methods, compositions, medicaments, therapeutic combinations, combinational therapies, and uses, described herein.

In some embodiments, Compound A is administered as a salt (e.g., methanesulfonate salt), at the amount and/or dosing frequencies described herein. In some embodiments, when Compound A is administered as a salt (e.g., methanesulfonate salt), the amount of the Compound A salt can be adjusted based on the amount of Compound A and the molecule weights of Compound A and the Compound A salt, such that the same mole amount is administered.

In some embodiments, Compound A is administered at an amount of about 1 mg to about 500 mg.

In some embodiments, Compound A is administered at about 1 mg to about 400 mg.

In some embodiments, Compound A is administered at about 1 mg to about 300 mg.

In some embodiments, Compound A is administered at about 5 mg to about 200 mg.

In some embodiments, Compound A is administered at about 10 mg to about 100 mg.

In some embodiments, Compound A is administered at about 10 mg to about 95 mg.

In some embodiments, Compound A is administered at about 10 mg to about 90 mg.

In some embodiments, Compound A is administered at about 10 mg to about 85 mg.

In some embodiments, Compound A is administered at about 10 mg to about 80 mg.

In some embodiments, Compound A is administered at about 10 mg to about 75 mg.

In some embodiments, Compound A is administered at about 10 mg to about 70 mg.

In some embodiments, Compound A is administered at about 10 mg to about 65 mg.

In some embodiments, Compound A is administered at about 10 mg to about 60 mg.

In some embodiments, Compound A is administered at about 10 mg to about 55 mg.

In some embodiments, Compound A is administered at about 10 mg to about 50 mg.

In some embodiments, Compound A is administered at about 10 mg to about 45 mg.

In some embodiments, Compound A is administered at about 10 mg to about 40 mg.

In some embodiments, Compound A is administered at about 10 mg to about 35 mg.

In some embodiments, Compound A is administered at about 10 mg to about 30 mg.

In some embodiments, Compound A is administered at about 10 mg to about 25 mg.

In some embodiments, Compound A is administered at about 10 mg to about 20 mg.

In some embodiments, Compound A is administered at about 10 mg to about 15 mg.

In some embodiments, Compound A is administered at about 15 mg to about 50 mg.

In some embodiments, Compound A is administered at about 15 mg to about 45 mg.

In some embodiments, Compound A is administered at about 15 mg to about 40 mg.

In some embodiments, Compound A is administered at about 15 mg to about 35 mg.

In some embodiments, Compound A is administered at about 15 mg to about 30 mg.

In some embodiments, Compound A is administered at about 15 mg to about 25 mg.

In some embodiments, Compound A is administered at about 15 mg to about 20 mg.

In some embodiments, Compound A is administered at about 5 mg.

In some embodiments, Compound A is administered at about 6 mg.

In some embodiments, Compound A is administered at about 7 mg.

In some embodiments, Compound A is administered at about 8 mg.

In some embodiments, Compound A is administered at about 9 mg.

In some embodiments, Compound A is administered at about 10 mg.

In some embodiments, Compound A is administered at about 11 mg.

In some embodiments, Compound A is administered at about 12 mg.

In some embodiments, Compound A is administered at about 12.7 mg.

In some embodiments, Compound A is administered at about 13 mg.

In some embodiments, Compound A is administered at about 14 mg.

In some embodiments, Compound A is administered at about 15 mg.

In some embodiments, Compound A is administered at about 20 mg.

In some embodiments, Compound A is administered at about 25 mg.

In some embodiments, Compound A is administered at about 30 mg

In some embodiments, Compound A is administered at about 35 mg.

In some embodiments, Compound A is administered at about 40 mg.

In some embodiments, Compound A is administered at about 45 mg.

In some embodiments, Compound A is administered at about 50 mg.

In some embodiments, Compound A in the present application is formulated for oral administration at the various doses described herein with the various dosing frequencies in the methods, compositions, medicaments, therapeutic combinations, combinational therapies, and uses, described herein.

In some embodiments, Compound A is administered on consecutive days.

In some embodiments, Compound A is administered 1-7 times per week.

In some embodiments, Compound A is administered 2-7 times per week.

In some embodiments, Compound A is administered 3-7 times per week.

In some embodiments, Compound A is administered 4-7 times per week.

In some embodiments, Compound A is administered 5-7 times per week.

In some embodiments, Compound A is administered 6-7 times per week.

In some embodiments, Compound A is administered 1-6 times per week. In some embodiments, Compound A is administered 1-5 times per week. In some embodiments, Compound A is administered 1-4 times per week. In some embodiments, Compound A is administered 1-3 times per week. In some embodiments, Compound A is administered 1-2 times per week.

In some embodiments, Compound A is administered 2-6 times per week. In some embodiments, Compound A is administered 2-5 times per week. In some embodiments, Compound A is administered 2-4 times per week. In some embodiments, Compound A is administered 2-3 times per week.

In some embodiments, Compound A is administered 3-6 times per week. In some embodiments, Compound A is administered 3-5 times per week. In some embodiments, Compound A is administered 3-4 times per week.

In some embodiments, Compound A is administered 4-6 times per week. In some embodiments, Compound A is administered 4-5 times per week.

In some embodiments, Compound A is administered 5-6 times per week.

In some embodiments, Compound A is administered at least 1 time per week.

In some embodiments, Compound A is administered at least 2 times per week. In some embodiments, Compound A is administered on consecutive days.

In some embodiments, Compound A is administered at least 3 times per week.

In some embodiments, Compound A is administered at least 4 times per week.

In some embodiments, Compound A is administered at least 5 times per week.

In some embodiments, Compound A is administered at least 6 times per week.

In some embodiments, Compound A is administered daily per week.

In some embodiments, Compound A is administered at least 1 time per week at an amount of about 1 mg to about 500 mg.

In some embodiments, Compound A is administered at least 1 time per week at an amount of about 1 mg to about 400 mg.

In some embodiments, Compound A is administered at least 1 time per week at an amount of about 1 mg to about 300 mg.

In some embodiments, Compound A is administered at least 1 time per week at an amount of about 5 mg to about 200 mg.

In some embodiments, Compound A is administered at least 1 time per week at an amount of about 10 mg to about 100 mg.

In some embodiments, Compound A is administered at least 1 time per week at an amount of about 10 mg to about 95 mg.

In some embodiments, Compound A is administered at least 1 time per week at an amount of about 10 mg to about 90 mg.

In some embodiments, Compound A is administered at least 1 time per week at an amount of about 10 mg to about 85 mg.

In some embodiments, Compound A is administered at least 1 time per week at an amount of about 10 mg to about 80 mg.

In some embodiments, Compound A is administered at least 1 time per week at an amount of about 10 mg to about 75 mg.

In some embodiments, Compound A is administered at least 1 time per week at an amount of about 10 mg to about 70 mg.

In some embodiments, Compound A is administered at least 1 time per week at an amount of about 10 mg to about 65 mg.

In some embodiments, Compound A is administered at least 1 time per week at an amount of about 10 mg to about 60 mg.

In some embodiments, Compound A is administered at least 1 time per week at an amount of about 10 mg to about 55 mg.

In some embodiments, Compound A is administered at least 1 time per week at an amount of about 10 mg to about 50 mg.

In some embodiments, Compound A is administered at least 1 time per week at an amount of about 10 mg to about 45 mg.

In some embodiments, Compound A is administered at least 1 time per week at an amount of about 10 mg to about 40 mg.

In some embodiments, Compound A is administered at least 1 time per week at an amount of about 10 mg to about 35 mg.

In some embodiments, Compound A is administered at least 1 time per week at an amount of about 10 mg to about 30 mg.

In some embodiments, Compound A is administered at least 1 time per week at an amount of about 10 mg to about 25 mg.

In some embodiments, Compound A is administered at least 1 time per week at an amount of about 10 mg to about 20 mg.

In some embodiments, Compound A is administered at least 1 time per week at an amount of about 10 mg to about 15 mg.

In some embodiments, Compound A is administered at least 1 time per week at an amount of about 15 mg to about 50 mg.

In some embodiments, Compound A is administered at least 1 time per week at an amount of about 15 mg to about 45 mg.

In some embodiments, Compound A is administered at least 1 time per week at an amount of about 15 mg to about 40 mg.

In some embodiments, Compound A is administered at least 1 time per week at an amount of about 15 mg to about 35 mg.

In some embodiments, Compound A is administered at least 1 time per week at an amount of about 15 mg to about 30 mg.

In some embodiments, Compound A is administered at least 1 time per week at an amount of about 15 mg to about 25 mg.

In some embodiments, Compound A is administered at least 1 time per week at an amount of about 15 mg to about 20 mg.

In some embodiments, Compound A is administered at least 1 time per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 1 time per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 1 time per week at an amount of about 55 mg, about 60 mg, about 65 mg, about 70 mg, or about 75 mg. In some embodiments, Compound A is administered at least 1 time per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 1 time per week at an amount of about 5 mg, about 10 mg, about 15 mg, about 20 mg, or about 25 mg. In some embodiments, Compound A is administered at least 1 time per week at an amount of about 30 mg, about 35 mg, about 40 mg, about 45 mg, or about 50 mg. In some embodiments, Compound A is administered at least 1 time per week at an amount of about 10 mg, about 15 mg, or about 20 mg. In some embodiments, Compound A is administered at least 1 time per week at an amount of about 15 mg.

In some embodiments, Compound A is administered at least 2 times per week at an amount of about 1 mg to about 500 mg.

In some embodiments, Compound A is administered at least 2 times per week at an amount of about 1 mg to about 400 mg.

In some embodiments, Compound A is administered at least 2 times per week at an amount of about 1 mg to about 300 mg.

In some embodiments, Compound A is administered at least 2 times per week at an amount of about 5 mg to about 200 mg.

In some embodiments, Compound A is administered at least 2 times per week at an amount of about 10 mg to about 100 mg.

In some embodiments, Compound A is administered at least 2 times per week at an amount of about 10 mg to about 95 mg.

In some embodiments, Compound A is administered at least 2 times per week at an amount of about 10 mg to about 90 mg.

In some embodiments, Compound A is administered at least 2 times per week at an amount of about 10 mg to about 85 mg.

In some embodiments, Compound A is administered at least 2 times per week at an amount of about 10 mg to about 80 mg.

In some embodiments, Compound A is administered at least 2 times per week at an amount of about 10 mg to about 75 mg.

In some embodiments, Compound A is administered at least 2 times per week at an amount of about 10 mg to about 70 mg.

In some embodiments, Compound A is administered at least 2 times per week at an amount of about 10 mg to about 65 mg.

In some embodiments, Compound A is administered at least 2 times per week at an amount of about 10 mg to about 60 mg.

In some embodiments, Compound A is administered at least 2 times per week at an amount of about 10 mg to about 55 mg.

In some embodiments, Compound A is administered at least 2 times per week at an amount of about 10 mg to about 50 mg.

In some embodiments, Compound A is administered at least 2 times per week at an amount of about 10 mg to about 45 mg.

In some embodiments, Compound A is administered at least 2 times per week at an amount of about 10 mg to about 40 mg.

In some embodiments, Compound A is administered at least 2 times per week at an amount of about 10 mg to about 35 mg.

In some embodiments, Compound A is administered at least 2 times per week at an amount of about 10 mg to about 30 mg.

In some embodiments, Compound A is administered at least 2 times per week at an amount of about 10 mg to about 25 mg.

In some embodiments, Compound A is administered at least 2 times per week at an amount of about 10 mg to about 20 mg.

In some embodiments, Compound A is administered at least 2 times per week at an amount of about 10 mg to about 15 mg.

In some embodiments, Compound A is administered at least 2 times per week at an amount of about 15 mg to about 50 mg.

In some embodiments, Compound A is administered at least 2 times per week at an amount of about 15 mg to about 45 mg.

In some embodiments, Compound A is administered at least 2 times per week at an amount of about 15 mg to about 40 mg.

In some embodiments, Compound A is administered at least 2 times per week at an amount of about 15 mg to about 35 mg.

In some embodiments, Compound A is administered at least 2 times per week at an amount of about 15 mg to about 30 mg.

In some embodiments, Compound A is administered at least 2 times per week at an amount of about 15 mg to about 25 mg.

In some embodiments, Compound A is administered at least 2 times per week at an amount of about 15 mg to about 20 mg.

In some embodiments, Compound A is administered at least 2 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 2 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 2 times per week at an amount of about 55 mg, about 60 mg, about 65 mg, about 70 mg, or about 75 mg. In some embodiments, Compound A is administered at least 2 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 2 times per week at an amount of about 5 mg, about 10 mg, about 15 mg, about 20 mg, or about 25 mg. In some embodiments, Compound A is administered at least 2 times per week at an amount of about 30 mg, about 35 mg, about 40 mg, about 45 mg, or about 50 mg. In some embodiments, Compound A is administered at least 2 times per week at an amount of about 10 mg, about 15 mg, or about 20 mg. In some embodiments, Compound A is administered at least 2 times per week at an amount of about 15 mg.

In some embodiments, Compound A is administered at least 3 times per week at an amount of about 1 mg to about 500 mg.

In some embodiments, Compound A is administered at least 3 times per week at an amount of about 1 mg to about 400 mg.

In some embodiments, Compound A is administered at least 3 times per week at an amount of about 1 mg to about 300 mg.

In some embodiments, Compound A is administered at least 3 times per week at an amount of about 5 mg to about 200 mg.

In some embodiments, Compound A is administered at least 3 times per week at an amount of about 10 mg to about 100 mg.

In some embodiments, Compound A is administered at least 3 times per week at an amount of about 10 mg to about 95 mg.

In some embodiments, Compound A is administered at least 3 times per week at an amount of about 10 mg to about 90 mg.

In some embodiments, Compound A is administered at least 3 times per week at an amount of about 10 mg to about 85 mg.

In some embodiments, Compound A is administered at least 3 times per week at an amount of about 10 mg to about 80 mg.

In some embodiments, Compound A is administered at least 3 times per week at an amount of about 10 mg to about 75 mg.

In some embodiments, Compound A is administered at least 3 times per week at an amount of about 10 mg to about 70 mg.

In some embodiments, Compound A is administered at least 3 times per week at an amount of about 10 mg to about 65 mg.

In some embodiments, Compound A is administered at least 3 times per week at an amount of about 10 mg to about 60 mg.

In some embodiments, Compound A is administered at least 3 times per week at an amount of about 10 mg to about 55 mg.

In some embodiments, Compound A is administered at least 3 times per week at an amount of about 10 mg to about 50 mg.

In some embodiments, Compound A is administered at least 3 times per week at an amount of about 10 mg to about 45 mg.

In some embodiments, Compound A is administered at least 3 times per week at an amount of about 10 mg to about 40 mg.

In some embodiments, Compound A is administered at least 3 times per week at an amount of about 10 mg to about 35 mg.

In some embodiments, Compound A is administered at least 3 times per week at an amount of about 10 mg to about 30 mg.

In some embodiments, Compound A is administered at least 3 times per week at an amount of about 10 mg to about 25 mg.

In some embodiments, Compound A is administered at least 3 times per week at an amount of about 10 mg to about 20 mg.

In some embodiments, Compound A is administered at least 3 times per week at an amount of about 10 mg to about 15 mg.

In some embodiments, Compound A is administered at least 3 times per week at an amount of about 15 mg to about 50 mg.

In some embodiments, Compound A is administered at least 3 times per week at an amount of about 15 mg to about 45 mg.

In some embodiments, Compound A is administered at least 3 times per week at an amount of about 15 mg to about 40 mg.

In some embodiments, Compound A is administered at least 3 times per week at an amount of about 15 mg to about 35 mg.

In some embodiments, Compound A is administered at least 3 times per week at an amount of about 15 mg to about 30 mg.

In some embodiments, Compound A is administered at least 3 times per week at an amount of about 15 mg to about 25 mg.

In some embodiments, Compound A is administered at least 3 times per week at an amount of about 15 mg to about 20 mg.

In some embodiments, Compound A is administered at least 3 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 3 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 3 times per week at an amount of about 55 mg, about 60 mg, about 65 mg, about 70 mg, or about 75 mg. In some embodiments, Compound A is administered at least 3 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 3 times per week at an amount of about 5 mg, about 10 mg, about 15 mg, about 20 mg, or about 25 mg. In some embodiments, Compound A is administered at least 3 times per week at an amount of about 30 mg, about 35 mg, about 40 mg, about 45 mg, or about 50 mg. In some embodiments, Compound A is administered at least 3 times per week at an amount of about 10 mg, about 15 mg, or about 20 mg. In some embodiments, Compound A is administered at least 3 times per week at an amount of about 15 mg.

In some embodiments, Compound A is administered at least 4 times per week at an amount of about 1 mg to about 500 mg.

In some embodiments, Compound A is administered at least 4 times per week at an amount of about 1 mg to about 400 mg.

In some embodiments, Compound A is administered at least 4 times per week at an amount of about 1 mg to about 300 mg.

In some embodiments, Compound A is administered at least 4 times per week at an amount of about 5 mg to about 200 mg.

In some embodiments, Compound A is administered at least 4 times per week at an amount of about 10 mg to about 100 mg.

In some embodiments, Compound A is administered at least 4 times per week at an amount of about 10 mg to about 95 mg.

In some embodiments, Compound A is administered at least 4 times per week at an amount of about 10 mg to about 90 mg.

In some embodiments, Compound A is administered at least 4 times per week at an amount of about 10 mg to about 85 mg.

In some embodiments, Compound A is administered at least 4 times per week at an amount of about 10 mg to about 80 mg.

In some embodiments, Compound A is administered at least 4 times per week at an amount of about 10 mg to about 75 mg.

In some embodiments, Compound A is administered at least 4 times per week at an amount of about 10 mg to about 70 mg.

In some embodiments, Compound A is administered at least 4 times per week at an amount of about 10 mg to about 65 mg.

In some embodiments, Compound A is administered at least 4 times per week at an amount of about 10 mg to about 60 mg.

In some embodiments, Compound A is administered at least 4 times per week at an amount of about 10 mg to about 55 mg.

In some embodiments, Compound A is administered at least 4 times per week at an amount of about 10 mg to about 50 mg.

In some embodiments, Compound A is administered at least 4 times per week at an amount of about 10 mg to about 45 mg.

In some embodiments, Compound A is administered at least 4 times per week at an amount of about 10 mg to about 40 mg.

In some embodiments, Compound A is administered at least 4 times per week at an amount of about 10 mg to about 35 mg.

In some embodiments, Compound A is administered at least 4 times per week at an amount of about 10 mg to about 30 mg.

In some embodiments, Compound A is administered at least 4 times per week at an amount of about 10 mg to about 25 mg.

In some embodiments, Compound A is administered at least 4 times per week at an amount of about 10 mg to about 20 mg.

In some embodiments, Compound A is administered at least 4 times per week at an amount of about 10 mg to about 15 mg.

In some embodiments, Compound A is administered at least 4 times per week at an amount of about 15 mg to about 50 mg.

In some embodiments, Compound A is administered at least 4 times per week at an amount of about 15 mg to about 45 mg.

In some embodiments, Compound A is administered at least 4 times per week at an amount of about 15 mg to about 40 mg.

In some embodiments, Compound A is administered at least 4 times per week at an amount of about 15 mg to about 35 mg.

In some embodiments, Compound A is administered at least 4 times per week at an amount of about 15 mg to about 30 mg.

In some embodiments, Compound A is administered at least 4 times per week at an amount of about 15 mg to about 25 mg.

In some embodiments, Compound A is administered at least 4 times per week at an amount of about 15 mg to about 20 mg.

In some embodiments, Compound A is administered at least 4 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 4 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 4 times per week at an amount of about 55 mg, about 60 mg, about 65 mg, about 70 mg, or about 75 mg. In some embodiments, Compound A is administered at least 4 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 4 times per week at an amount of about 5 mg, about 10 mg, about 15 mg, about 20 mg, or about 25 mg. In some embodiments, Compound A is administered at least 4 times per week at an amount of about 30 mg, about 35 mg, about 40 mg, about 45 mg, or about 50 mg. In some embodiments, Compound A is administered at least 4 times per week at an amount of about 10 mg, about 15 mg, or about 20 mg. In some embodiments, Compound A is administered at least 4 times per week at an amount of about 15 mg.

In some embodiments, Compound A is administered at least 5 times per week at an amount of about 1 mg to about 500 mg.

In some embodiments, Compound A is administered at least 5 times per week at an amount of about 1 mg to about 400 mg.

In some embodiments, Compound A is administered at least 5 times per week at an amount of about 1 mg to about 300 mg.

In some embodiments, Compound A is administered at least 5 times per week at an amount of about 5 mg to about 200 mg.

In some embodiments, Compound A is administered at least 5 times per week at an amount of about 10 mg to about 100 mg.

In some embodiments, Compound A is administered at least 5 times per week at an amount of about 10 mg to about 95 mg.

In some embodiments, Compound A is administered at least 5 times per week at an amount of about 10 mg to about 90 mg.

In some embodiments, Compound A is administered at least 5 times per week at an amount of about 10 mg to about 85 mg.

In some embodiments, Compound A is administered at least 5 times per week at an amount of about 10 mg to about 80 mg.

In some embodiments, Compound A is administered at least 5 times per week at an amount of about 10 mg to about 75 mg.

In some embodiments, Compound A is administered at least 5 times per week at an amount of about 10 mg to about 70 mg.

In some embodiments, Compound A is administered at least 5 times per week at an amount of about 10 mg to about 65 mg.

In some embodiments, Compound A is administered at least 5 times per week at an amount of about 10 mg to about 60 mg.

In some embodiments, Compound A is administered at least 5 times per week at an amount of about 10 mg to about 55 mg.

In some embodiments, Compound A is administered at least 5 times per week at an amount of about 10 mg to about 50 mg.

In some embodiments, Compound A is administered at least 5 times per week at an amount of about 10 mg to about 45 mg.

In some embodiments, Compound A is administered at least 5 times per week at an amount of about 10 mg to about 40 mg.

In some embodiments, Compound A is administered at least 5 times per week at an amount of about 10 mg to about 35 mg.

In some embodiments, Compound A is administered at least 5 times per week at an amount of about 10 mg to about 30 mg.

In some embodiments, Compound A is administered at least 5 times per week at an amount of about 10 mg to about 25 mg.

In some embodiments, Compound A is administered at least 5 times per week at an amount of about 10 mg to about 20 mg.

In some embodiments, Compound A is administered at least 5 times per week at an amount of about 10 mg to about 15 mg.

In some embodiments, Compound A is administered at least 5 times per week at an amount of about 15 mg to about 50 mg.

In some embodiments, Compound A is administered at least 5 times per week at an amount of about 15 mg to about 45 mg.

In some embodiments, Compound A is administered at least 5 times per week at an amount of about 15 mg to about 40 mg.

In some embodiments, Compound A is administered at least 5 times per week at an amount of about 15 mg to about 35 mg.

In some embodiments, Compound A is administered at least 5 times per week at an amount of about 15 mg to about 30 mg.

In some embodiments, Compound A is administered at least 5 times per week at an amount of about 15 mg to about 25 mg.

In some embodiments, Compound A is administered at least 5 times per week at an amount of about 15 mg to about 20 mg.

In some embodiments, Compound A is administered at least 5 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 5 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 5 times per week at an amount of about 55 mg, about 60 mg, about 65 mg, about 70 mg, or about 75 mg. In some embodiments, Compound A is administered at least 5 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 5 times per week at an amount of about 5 mg, about 10 mg, about 15 mg, about 20 mg, or about 25 mg. In some embodiments, Compound A is administered at least 5 times per week at an amount of about 30 mg, about 35 mg, about 40 mg, about 45 mg, or about 50 mg. In some embodiments, Compound A is administered at least 5 times per week at an amount of about 10 mg, about 15 mg, or about 20 mg. In some embodiments, Compound A is administered at least 5 times per week at an amount of about 15 mg.

In some embodiments, Compound A is administered at least 6 times per week at an amount of about 1 mg to about 500 mg.

In some embodiments, Compound A is administered at least 6 times per week at an amount of about 1 mg to about 400 mg.

In some embodiments, Compound A is administered at least 6 times per week at an amount of about 1 mg to about 300 mg.

In some embodiments, Compound A is administered at least 6 times per week at an amount of about 5 mg to about 200 mg.

In some embodiments, Compound A is administered at least 6 times per week at an amount of about 10 mg to about 100 mg.

In some embodiments, Compound A is administered at least 6 times per week at an amount of about 10 mg to about 95 mg.

In some embodiments, Compound A is administered at least 6 times per week at an amount of about 10 mg to about 90 mg.

In some embodiments, Compound A is administered at least 6 times per week at an amount of about 10 mg to about 85 mg.

In some embodiments, Compound A is administered at least 6 times per week at an amount of about 10 mg to about 80 mg.

In some embodiments, Compound A is administered at least 6 times per week at an amount of about 10 mg to about 75 mg.

In some embodiments, Compound A is administered at least 6 times per week at an amount of about 10 mg to about 70 mg.

In some embodiments, Compound A is administered at least 6 times per week at an amount of about 10 mg to about 65 mg.

In some embodiments, Compound A is administered at least 6 times per week at an amount of about 10 mg to about 60 mg.

In some embodiments, Compound A is administered at least 6 times per week at an amount of about 10 mg to about 55 mg.

In some embodiments, Compound A is administered at least 6 times per week at an amount of about 10 mg to about 50 mg.

In some embodiments, Compound A is administered at least 6 times per week at an amount of about 10 mg to about 45 mg.

In some embodiments, Compound A is administered at least 6 times per week at an amount of about 10 mg to about 40 mg.

In some embodiments, Compound A is administered at least 6 times per week at an amount of about 10 mg to about 35 mg.

In some embodiments, Compound A is administered at least 6 times per week at an amount of about 10 mg to about 30 mg.

In some embodiments, Compound A is administered at least 6 times per week at an amount of about 10 mg to about 25 mg.

In some embodiments, Compound A is administered at least 6 times per week at an amount of about 10 mg to about 20 mg.

In some embodiments, Compound A is administered at least 6 times per week at an amount of about 10 mg to about 15 mg.

In some embodiments, Compound A is administered at least 6 times per week at an amount of about 15 mg to about 50 mg.

In some embodiments, Compound A is administered at least 6 times per week at an amount of about 15 mg to about 45 mg.

In some embodiments, Compound A is administered at least 6 times per week at an amount of about 15 mg to about 40 mg.

In some embodiments, Compound A is administered at least 6 times per week at an amount of about 15 mg to about 35 mg.

In some embodiments, Compound A is administered at least 6 times per week at an amount of about 15 mg to about 30 mg.

In some embodiments, Compound A is administered at least 6 times per week at an amount of about 15 mg to about 25 mg.

In some embodiments, Compound A is administered at least 6 times per week at an amount of about 15 mg to about 20 mg.

In some embodiments, Compound A is administered at least 6 times per week at an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, Compound A is administered at least 6 times per week at an amount of about 55 mg, about 60 mg, about 65 mg, about 70 mg, about 75 mg, about 80 mg, about 85 mg, about 90 mg, about 95 mg, or about 100 mg. In some embodiments, Compound A is administered at least 6 times per week at an amount of about 55 mg, about 60 mg, about 65 mg, about 70 mg, or about 75 mg. In some embodiments, Compound A is administered at least 6 times per week at an amount of about 5 mg, about 10 mg, about 15 mg, about 20 mg, about 25 mg, about 30 mg, about 35 mg, about 40 mg, about 45 mg, or about 50 mg. In some embodiments, Compound A is administered at least 6 times per week at an amount of about 5 mg, about 10 mg, about 15 mg, about 20 mg, or about 25 mg. In some embodiments, Compound A is administered at least 6 times per week at an amount of about 30 mg, about 35 mg, about 40 mg, about 45 mg, or about 50 mg. In some embodiments, Compound A is administered at least 6 times per week at an amount of about 10 mg, about 15 mg, or about 20 mg. In some embodiments, Compound A is administered at least 6 times per week at an amount of about 15 mg.

In some embodiments, Compound A is administered for a three week cycle.

In some embodiments, Compound A is administered for one week of a three week cycle.

In some embodiments, Compound A is administered at least once a week for one week of a three week cycle.

In some embodiments, Compound A is administered at least twice a week for one week of a three week cycle.

In some embodiments, Compound A is administered at least three times a week for one week of a three week cycle.

In some embodiments, Compound A is administered at least four times a week for one week of a three week cycle.

In some embodiments, Compound A is administered at least five times a week for one week of a three week cycle.

In some embodiments, Compound A is administered at least six times a week for one week of a three week cycle.

In some embodiments, Compound A is administered at least daily for one week of a three week cycle.

In some embodiments, Compound A is administered at least once a week for one week of a three week cycle from about 1 mg to about 500 mg.

In some embodiments, Compound A is administered at least twice a week for one week of a three week cycle from about 1 mg to about 500 mg.

In some embodiments, Compound A is administered at least three times a week for one week of a three week cycle from about 1 mg to about 500 mg.

In some embodiments, Compound A is administered at least four times a week for one week of a three week cycle from about 1 mg to about 500 mg.

In some embodiments, Compound A is administered at least five times a week for one week of a three week cycle from about 1 mg to about 500 mg.

In some embodiments, Compound A is administered at least six times a week for one week of a three week cycle from about 1 mg to about 500 mg.

In some embodiments, Compound A is administered at least daily for one week of a three week cycle from about 1 mg to about 500 mg.

In some embodiments, Compound A is administered at least once a week for one week of a three week cycle from about 10 mg to about 50 mg.

In some embodiments, Compound A is administered at least twice a week for one week of a three week cycle from about 10 mg to about 50 mg.

In some embodiments, Compound A is administered at least three times a week for one week of a three week cycle from about 10 mg to about 50 mg.

In some embodiments, Compound A is administered at least four times a week for one week of a three week cycle from about 10 mg to about 50 mg.

In some embodiments, Compound A is administered at least five times a week for one week of a three week cycle from about 10 mg to about 50 mg.

In some embodiments, Compound A is administered at least six times a week for one week of a three week cycle from about 10 mg to about 50 mg.

In some embodiments, Compound A is administered at least daily for one week of a three week cycle from about 10 mg to about 400 mg.

In some embodiments, Compound A is administered at least once a week for one week of a three week cycle at about 15 mg.

In some embodiments, Compound A is administered at least twice a week for one week of a three week cycle at about 15 mg.

In some embodiments, Compound A is administered at least three times a week for one week of a three week cycle at about 15 mg.

In some embodiments, Compound A is administered at least four times a week for one week of a three week cycle at about 15 mg.

In some embodiments, Compound A is administered at least five times a week for one week of a three week cycle at about 15 mg.

In some embodiments, Compound A is administered at least six times a week for one week of a three week cycle at about 15 mg.

In some embodiments, Compound A is administered at least daily for one week of a three week cycle from at about 15 mg.

In some embodiments, Compound A is administered for two weeks of a three week cycle.

In some embodiments, Compound A is administered at least once a week for two weeks of a three week cycle.

In some embodiments, Compound A is administered at least twice a week for two weeks of a three week cycle.

In some embodiments, Compound A is administered at least three times a week for two weeks of a three week cycle.

In some embodiments, Compound A is administered at least four times a week for two weeks of a three week cycle.

In some embodiments, Compound A is administered at least five times a week for two weeks of a three week cycle.

In some embodiments, Compound A is administered at least six times a week for two weeks of a three week cycle.

In some embodiments, Compound A is administered at least daily for two weeks of a three week cycle.

In some embodiments, Compound A is administered at least once a week for two weeks of a three week cycle from about 1 mg to about 500 mg.

In some embodiments, Compound A is administered at least twice a week for two weeks of a three week cycle from about 1 mg to about 500 mg.

In some embodiments, Compound A is administered at least three times a week for two weeks of a three week cycle from about 1 mg to about 500 mg.

In some embodiments, Compound A is administered at least four times a week for two weeks of a three week cycle from about 1 mg to about 500 mg.

In some embodiments, Compound A is administered at least five times a week for two weeks of a three week cycle from about 1 mg to about 500 mg.

In some embodiments, Compound A is administered at least six times a week for two weeks of a three week cycle from about 1 mg to about 500 mg.

In some embodiments, Compound A is administered at least daily for two weeks of a three week cycle from about 1 mg to about 500 mg.

In some embodiments, Compound A is administered at least once a week for two weeks of a three week cycle from about 10 mg to about 50 mg.

In some embodiments, Compound A is administered at least twice a week for two weeks of a three week cycle from about 10 mg to about 50 mg.

In some embodiments, Compound A is administered at least three times a week for two weeks of a three week cycle from about 10 mg to about 50 mg.

In some embodiments, Compound A is administered at least four times a week for two weeks of a three week cycle from about 10 mg to about 50 mg.

In some embodiments, Compound A is administered at least five times a week for two weeks of a three week cycle from about 10 mg to about 50 mg.

In some embodiments, Compound A is administered at least six times a week for two weeks of a three week cycle from about 10 mg to about 50 mg.

In some embodiments, Compound A is administered at least daily for two weeks of a three week cycle from about 10 mg to about 50 mg.

In some embodiments, Compound A is administered at least once a week for two weeks of a three week cycle at about 15 mg.

In some embodiments, Compound A is administered at least twice a week for two weeks of a three week cycle at about 15 mg.

In some embodiments, Compound A is administered at least three times a week for two weeks of a three week cycle at about 15 mg.

In some embodiments, Compound A is administered at least four times a week for two weeks of a three week cycle at about 15 mg.

In some embodiments, Compound A is administered at least five times a week for two weeks of a three week cycle at about 15 mg.

In some embodiments, Compound A is administered at least six times a week for two weeks of a three week cycle at about 15 mg.

In some embodiments, Compound A is administered at least daily for two weeks of a three week cycle at about 15 mg.

In some embodiments, Compound A is administered for three weeks of a three week cycle.

In some embodiments, Compound A is administered at least once a week for three weeks of a three week cycle.

In some embodiments, Compound A is administered at least twice a week for three weeks of a three week cycle.

In some embodiments, Compound A is administered at least three times a week for three weeks of a three week cycle.

In some embodiments, Compound A is administered at least four times a week for three weeks of a three week cycle.

In some embodiments, Compound A is administered at least five times a week for three weeks of a three week cycle.

In some embodiments, Compound A is administered at least six times a week for three weeks of a three week cycle.

In some embodiments, Compound A is administered at least daily for three weeks of a three week cycle.

In some embodiments, Compound A is administered at least once a week for three weeks of a three week cycle from about 1 mg to about 500 mg.

In some embodiments, Compound A is administered at least twice a week for three weeks of a three week cycle from about 1 mg to about 500 mg.

In some embodiments, Compound A is administered at least three times a week for three weeks of a three week cycle from about 1 mg to about 500 mg.

In some embodiments, Compound A is administered at least four times a week for three weeks of a three week cycle from about 1 mg to about 500 mg.

In some embodiments, Compound A is administered at least five times a week for three weeks of a three week cycle from about 1 mg to about 500 mg.

In some embodiments, Compound A is administered at least six times a week for three weeks of a three week cycle from about 1 mg to about 500 mg.

In some embodiments, Compound A is administered at least daily for three weeks of a three week cycle from about 1 mg to about 500 mg.

In some embodiments, Compound A is administered at least once a week for three weeks of a three week cycle from about 10 mg to about 50 mg.

In some embodiments, Compound A is administered at least twice a week for three weeks of a three week cycle from about 10 mg to about 50 mg.

In some embodiments, Compound A is administered at least three times a week for three weeks of a three week cycle from about 10 mg to about 50 mg.

In some embodiments, Compound A is administered at least four times a week for three weeks of a three week cycle from about 10 mg to about 50 mg.

In some embodiments, Compound A is administered at least five times a week for three weeks of a three week cycle from about 10 mg to about 50 mg.

In some embodiments, Compound A is administered at least six times a week for three weeks of a three week cycle from about 10 mg to about 50 mg.

In some embodiments, Compound A is administered at least daily for three weeks of a three week cycle from about 10 mg to about 50 mg.

In some embodiments, Compound A is administered at least once a week for three weeks of a three week cycle at about 15 mg.

In some embodiments, Compound A is administered at least twice a week for three weeks of a three week cycle at about 15 mg.

In some embodiments, Compound A is administered at least three times a week for three weeks of a three week cycle at about 15 mg.

In some embodiments, Compound A is administered at least four times a week for three weeks of a three week cycle at about 15 mg.

In some embodiments, Compound A is administered at least five times a week for three weeks of a three week cycle at about 15 mg.

In some embodiments, Compound A is administered at least six times a week for three weeks of a three week cycle at about 15 mg.

In some embodiments, Compound A is administered at least daily for three weeks of a three week cycle at about 15 mg.

### Checkpoint Inhibitor

In some embodiments, the checkpoint inhibitor in the present application is formulated for intravenous administration at the various doses in the methods, compositions, medicaments, therapeutic combinations, combinational therapies, and uses, described herein.

In some embodiments, the checkpoint inhibitor is administered at an amount of about 1 mg to about 500 mg.

In some embodiments, the checkpoint inhibitor is administered at an amount of about 1 mg to about 400 mg.

In some embodiments, the checkpoint inhibitor is administered at an amount of about 1 mg to about 300 mg.

In some embodiments, the checkpoint inhibitor is administered at an amount of about 1 mg to about 200 mg.

In some embodiments, the checkpoint inhibitor is administered at an amount of about 100 mg to about 500 mg.

In some embodiments, the checkpoint inhibitor is administered at an amount of about 100 mg to about 450 mg.

In some embodiments, the checkpoint inhibitor is administered at an amount of about 100 mg to about 400 mg.

In some embodiments, the checkpoint inhibitor is administered at an amount of about 100 mg to about 350 mg.

In some embodiments, the checkpoint inhibitor is administered at an amount of about 100 mg to about 300 mg.

In some embodiments, the checkpoint inhibitor is administered at an amount of about 100 mg to about 290 mg.

In some embodiments, the checkpoint inhibitor is administered at an amount of about 100 mg to about 280 mg.

In some embodiments, the checkpoint inhibitor is administered at an amount of about 100 mg to about 270 mg.

In some embodiments, the checkpoint inhibitor is administered at an amount of about 100 mg to about 260 mg.

In some embodiments, the checkpoint inhibitor is administered at an amount of about 100 mg to about 250 mg.

In some embodiments, the checkpoint inhibitor is administered at an amount of about 100 mg to about 240 mg.

In some embodiments, the checkpoint inhibitor is administered at an amount of about 100 mg to about 230 mg.

In some embodiments, the checkpoint inhibitor is administered at an amount of about 100 mg to about 220 mg.

In some embodiments, the checkpoint inhibitor is administered at an amount of about 100 mg to about 210 mg.

In some embodiments, the checkpoint inhibitor is administered at an amount of about 100 mg to about 200 mg.

In some embodiments, the checkpoint inhibitor is administered at an amount of about 100 mg to about 190 mg.

In some embodiments, the checkpoint inhibitor is administered at an amount of about 100 mg to about 180 mg.

In some embodiments, the checkpoint inhibitor is administered at an amount of about 100 mg to about 170 mg.

In some embodiments, the checkpoint inhibitor is administered at an amount of about 100 mg to about 160 mg.

In some embodiments, the checkpoint inhibitor is administered at an amount of about 100 mg to about 150 mg.

In some embodiments, the checkpoint inhibitor is administered at an amount of about 100 mg to about 140 mg.

In some embodiments, the checkpoint inhibitor is administered at an amount of about 100 mg to about 130 mg.

In some embodiments, the checkpoint inhibitor is administered at an amount of about 100 mg to about 120 mg.

In some embodiments, the checkpoint inhibitor is administered at an amount of about 100 mg to about 110 mg.

In some embodiments, the checkpoint inhibitor is administered at an amount of about 150 mg to about 500 mg.

In some embodiments, the checkpoint inhibitor is administered at an amount of about 150 mg to about 450 mg.

In some embodiments, the checkpoint inhibitor is administered at an amount of about 150 mg to about 400 mg.

In some embodiments, the checkpoint inhibitor is administered at an amount of about 150 mg to about 350 mg.

In some embodiments, the checkpoint inhibitor is administered at an amount of about 150 mg to about 300 mg.

In some embodiments, the checkpoint inhibitor is administered at an amount of about 150 mg to about 290 mg.

In some embodiments, the checkpoint inhibitor is administered at an amount of about 150 mg to about 280 mg.

In some embodiments, the checkpoint inhibitor is administered at an amount of about 150 mg to about 270 mg.

In some embodiments, the checkpoint inhibitor is administered at an amount of about 150 mg to about 260 mg.

In some embodiments, the checkpoint inhibitor is administered at an amount of about 150 mg to about 250 mg.

In some embodiments, the checkpoint inhibitor is administered at an amount of about 150 mg to about 240 mg.

In some embodiments, the checkpoint inhibitor is administered at an amount of about 150 mg to about 230 mg.

In some embodiments, the checkpoint inhibitor is administered at an amount of about 150 mg to about 220 mg.

In some embodiments, the checkpoint inhibitor is administered at an amount of about 150 mg to about 210 mg.

In some embodiments, the checkpoint inhibitor is administered at an amount of about 150 mg to about 200 mg.

In some embodiments, the checkpoint inhibitor is administered at an amount of about 150 mg to about 190 mg.

In some embodiments, the checkpoint inhibitor is administered at an amount of about 150 mg to about 180 mg.

In some embodiments, the checkpoint inhibitor is administered at an amount of about 150 mg to about 170 mg.

In some embodiments, the checkpoint inhibitor is administered at an amount of about 150 mg to about 160 mg.

In some embodiments, the checkpoint inhibitor is administered at an amount of about 200 mg to about 500 mg.

In some embodiments, the checkpoint inhibitor is administered at an amount of about 200 mg to about 450 mg.

In some embodiments, the checkpoint inhibitor is administered at an amount of about 200 mg to about 400 mg.

In some embodiments, the checkpoint inhibitor is administered at an amount of about 200 mg to about 350 mg.

In some embodiments, the checkpoint inhibitor is administered at an amount of about 200 mg to about 300 mg.

In some embodiments, the checkpoint inhibitor is administered at an amount of about 200 mg to about 290 mg.

In some embodiments, the checkpoint inhibitor is administered at an amount of about 200 mg to about 280 mg.

In some embodiments, the checkpoint inhibitor is administered at an amount of about 200 mg to about 270 mg.

In some embodiments, the checkpoint inhibitor is administered at an amount of about 200 mg to about 260 mg.

In some embodiments, the checkpoint inhibitor is administered at an amount of about 200 mg to about 250 mg.

In some embodiments, the checkpoint inhibitor is administered at an amount of about 200 mg to about 240 mg.

In some embodiments, the checkpoint inhibitor is administered at an amount of about 200 mg to about 230 mg.

In some embodiments, the checkpoint inhibitor is administered at an amount of about 200 mg to about 220 mg.

In some embodiments, the checkpoint inhibitor is administered at an amount of about 200 mg to about 210 mg.

In some embodiments, the checkpoint inhibitor is administered at about 500 mg.

In some embodiments, the checkpoint inhibitor is administered at about 450 mg.

In some embodiments, the checkpoint inhibitor is administered at about 440 mg.

In some embodiments, the checkpoint inhibitor is administered at about 430 mg.

In some embodiments, the checkpoint inhibitor is administered at about 420 mg.

In some embodiments, the checkpoint inhibitor is administered at about 410 mg.

In some embodiments, the checkpoint inhibitor is administered at about 400 mg.

In some embodiments, the checkpoint inhibitor is administered at about 390 mg.

In some embodiments, the checkpoint inhibitor is administered at about 380 mg.

In some embodiments, the checkpoint inhibitor is administered at about 370 mg.

In some embodiments, the checkpoint inhibitor is administered at about 360 mg.

In some embodiments, the checkpoint inhibitor is administered at about 350 mg.

In some embodiments, the checkpoint inhibitor is administered at about 340 mg.

In some embodiments, the checkpoint inhibitor is administered at about 330 mg.

In some embodiments, the checkpoint inhibitor is administered at about 320 mg In some embodiments, the checkpoint inhibitor is administered at about 310 mg.

In some embodiments, the checkpoint inhibitor is administered at about 300 mg.

In some embodiments, the checkpoint inhibitor is administered at about 290 mg.

In some embodiments, the checkpoint inhibitor is administered at about 280 mg.

In some embodiments, the checkpoint inhibitor is administered at about 270 mg.

In some embodiments, the checkpoint inhibitor is administered at about 260 mg.

In some embodiments, the checkpoint inhibitor is administered at about 250 mg.

In some embodiments, the checkpoint inhibitor is administered at about 240 mg.

In some embodiments, the checkpoint inhibitor is administered at about 230 mg.

In some embodiments, the checkpoint inhibitor is administered at about 220 mg In some embodiments, the checkpoint inhibitor is administered at about 210 mg.

In some embodiments, the checkpoint inhibitor is administered at about 200 mg.

In some embodiments, the checkpoint inhibitor is administered at about 190 mg.

In some embodiments, the checkpoint inhibitor is administered at about 180 mg.

In some embodiments, the checkpoint inhibitor is administered at about 170 mg.

In some embodiments, the checkpoint inhibitor is administered at about 160 mg.

In some embodiments, the checkpoint inhibitor is administered at about 150 mg.

In some embodiments, the checkpoint inhibitor is administered at about 140 mg.

In some embodiments, the checkpoint inhibitor is administered at about 130 mg.

In some embodiments, the checkpoint inhibitor is administered at about 120 mg In some embodiments, the checkpoint inhibitor is administered at about 110 mg.

In some embodiments, the checkpoint inhibitor is administered at about 100 mg.

In some embodiments, the checkpoint inhibitor is administered at about 50 mg.

In some embodiments, the checkpoint inhibitor in the present application is formulated for intravenous administration at the various doses described herein with the various dosing frequencies in the methods, compositions, medicaments, therapeutic combinations, combinational therapies, and uses, described herein.

In some embodiments, the checkpoint inhibitor is administered on consecutive days.

In some embodiments, the checkpoint inhibitor is administered 1-7 times per week.

In some embodiments, the checkpoint inhibitor is administered 2-7 times per week.

In some embodiments, the checkpoint inhibitor is administered 3-7 times per week.

In some embodiments, the checkpoint inhibitor is administered 4-7 times per week.

In some embodiments, the checkpoint inhibitor is administered 5-7 times per week.

In some embodiments, the checkpoint inhibitor is administered 6-7 times per week.

In some embodiments, the checkpoint inhibitor is administered 1-6 times per week. In some embodiments, the checkpoint inhibitor is administered 1-5 times per week. In some embodiments, the checkpoint inhibitor is administered 1-4 times per week. In some embodiments, the checkpoint inhibitor is administered 1-3 times per week. In some embodiments, the checkpoint inhibitor is administered 1-2 times per week.

In some embodiments, the checkpoint inhibitor is administered 2-6 times per week. In some embodiments, the checkpoint inhibitor is administered 2-5 times per week. In some embodiments, the checkpoint inhibitor is administered 2-4 times per week. In some embodiments, the checkpoint inhibitor is administered 2-3 times per week.

In some embodiments, the checkpoint inhibitor is administered 3-6 times per week. In some embodiments, the checkpoint inhibitor is administered 3-5 times per week. In some embodiments, the checkpoint inhibitor is administered 3-4 times per week.

In some embodiments, the checkpoint inhibitor is administered 4-6 times per week. In some embodiments, the checkpoint inhibitor is administered 4-5 times per week.

In some embodiments, the checkpoint inhibitor is administered 5-6 times per week.

In some embodiments, the checkpoint inhibitor is administered at least 1 time per week.

In some embodiments, the checkpoint inhibitor is administered at least 2 times per week.

In some embodiments, the checkpoint inhibitor is administered at least 3 times per week.

In some embodiments, the checkpoint inhibitor is administered at least 4 times per week.

In some embodiments, the checkpoint inhibitor is administered at least 5 times per week.

In some embodiments, the checkpoint inhibitor is administered at least 6 times per week.

In some embodiments, the checkpoint inhibitor is administered daily per week.

In some embodiments, the checkpoint inhibitor is administered at least 1 time per week at an amount of about 1 mg to about 500 mg.

In some embodiments, the checkpoint inhibitor is administered at least 1 time per week at an amount of about 1 mg to about 400 mg.

In some embodiments, the checkpoint inhibitor is administered at least 1 time per week at an amount of about 1 mg to about 300 mg.

In some embodiments, the checkpoint inhibitor is administered at least 1 time per week at an amount of about 1 mg to about 200 mg.

In some embodiments, the checkpoint inhibitor is administered at least 1 time per week at an amount of about 10 mg to about 500 mg.

In some embodiments, the checkpoint inhibitor is administered at least 1 time per week at an amount of about 200 mg to about 500 mg.

In some embodiments, the checkpoint inhibitor is administered at least 1 time per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 1 time per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 1 time per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 1 time per week at an amount of about 400 mg. In some embodiments, the checkpoint inhibitor is administered at least 1 time per week at an amount of about 200 mg.

In some embodiments, the checkpoint inhibitor is administered at least 2 times per week at an amount of about 1 mg to about 500 mg.

In some embodiments, the checkpoint inhibitor is administered at least 2 times per week at an amount of about 1 mg to about 400 mg.

In some embodiments, the checkpoint inhibitor is administered at least 2 times per week at an amount of about 1 mg to about 300 mg.

In some embodiments, the checkpoint inhibitor is administered at least 2 times per week at an amount of about 1 mg to about 200 mg.

In some embodiments, the checkpoint inhibitor is administered at least 2 times per week at an amount of about 10 mg to about 500 mg.

In some embodiments, the checkpoint inhibitor is administered at least 2 times per week at an amount of about 200 mg to about 500 mg.

In some embodiments, the checkpoint inhibitor is administered at least 2 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 2 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 2 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 2 times per week at an amount of about 400 mg. In some embodiments, the checkpoint inhibitor is administered at least 2 times per week at an amount of about 200 mg.

In some embodiments, the checkpoint inhibitor is administered at least 3 times per week at an amount of about 1 mg to about 500 mg.

In some embodiments, the checkpoint inhibitor is administered at least 3 times per week at an amount of about 1 mg to about 400 mg.

In some embodiments, the checkpoint inhibitor is administered at least 3 times per week at an amount of about 1 mg to about 300 mg.

In some embodiments, the checkpoint inhibitor is administered at least 3 times per week at an amount of about 1 mg to about 200 mg.

In some embodiments, the checkpoint inhibitor is administered at least 3 times per week at an amount of about 10 mg to about 500 mg.

In some embodiments, the checkpoint inhibitor is administered at least 3 times per week at an amount of about 200 mg to about 500 mg.

In some embodiments, the checkpoint inhibitor is administered at least 3 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 3 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 3 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 3 times per week at an amount of about 400 mg. In some embodiments, the checkpoint inhibitor is administered at least 3 times per week at an amount of about 200 mg.

In some embodiments, the checkpoint inhibitor is administered at least 4 times per week at an amount of about 1 mg to about 500 mg.

In some embodiments, the checkpoint inhibitor is administered at least 4 times per week at an amount of about 1 mg to about 400 mg.

In some embodiments, the checkpoint inhibitor is administered at least 4 times per week at an amount of about 1 mg to about 300 mg.

In some embodiments, the checkpoint inhibitor is administered at least 4 times per week at an amount of about 1 mg to about 200 mg.

In some embodiments, the checkpoint inhibitor is administered at least 4 times per week at an amount of about 10 mg to about 500 mg.

In some embodiments, the checkpoint inhibitor is administered at least 4 times per week at an amount of about 200 mg to about 500 mg.

In some embodiments, the checkpoint inhibitor is administered at least 4 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 4 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 4 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 4 times per week at an amount of about 400 mg. In some embodiments, the checkpoint inhibitor is administered at least 4 times per week at an amount of about 200 mg.

In some embodiments, the checkpoint inhibitor is administered at least 5 times per week at an amount of about 1 mg to about 500 mg.

In some embodiments, the checkpoint inhibitor is administered at least 5 times per week at an amount of about 1 mg to about 400 mg.

In some embodiments, the checkpoint inhibitor is administered at least 5 times per week at an amount of about 1 mg to about 300 mg.

In some embodiments, the checkpoint inhibitor is administered at least 5 times per week at an amount of about 1 mg to about 200 mg.

In some embodiments, the checkpoint inhibitor is administered at least 5 times per week at an amount of about 10 mg to about 500 mg.

In some embodiments, the checkpoint inhibitor is administered at least 5 times per week at an amount of about 200 mg to about 500 mg.

In some embodiments, the checkpoint inhibitor is administered at least 5 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 5 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 5 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 5 times per week at an amount of about 400 mg. In some embodiments, the checkpoint inhibitor is administered at least 5 times per week at an amount of about 200 mg.

In some embodiments, the checkpoint inhibitor is administered at least 6 times per week at an amount of about 1 mg to about 500 mg.

In some embodiments, the checkpoint inhibitor is administered at least 6 times per week at an amount of about 1 mg to about 400 mg.

In some embodiments, the checkpoint inhibitor is administered at least 6 times per week at an amount of about 1 mg to about 300 mg.

In some embodiments, the checkpoint inhibitor is administered at least 6 times per week at an amount of about 1 mg to about 200 mg.

In some embodiments, the checkpoint inhibitor is administered at least 6 times per week at an amount of about 10 mg to about 500 mg.

In some embodiments, the checkpoint inhibitor is administered at least 6 times per week at an amount of about 200 mg to about 500 mg.

In some embodiments, the checkpoint inhibitor is administered at least 6 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, the checkpoint inhibitor is administered at least 6 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, the checkpoint inhibitor is administered at least 6 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, the checkpoint inhibitor is administered at least 6 times per week at an amount of about 400 mg. In some embodiments, the checkpoint inhibitor is administered at least 6 times per week at an amount of about 200 mg.

In some embodiments, the checkpoint inhibitor is administered once every three weeks.

In some embodiments, the checkpoint inhibitor is administered once every six weeks.

In some embodiments, the checkpoint inhibitor is administered once every three weeks from about 1 mg to about 500 mg.

In some embodiments, the checkpoint inhibitor is administered once every six weeks about 1 mg to about 500 mg.

In some embodiments, the checkpoint inhibitor is administered once every three weeks from about 1 mg to about 400 mg.

In some embodiments, the checkpoint inhibitor is administered once every six weeks from about 1 mg to about 400 mg.

In some embodiments, the checkpoint inhibitor is administered once every three weeks from about 1 mg to about 300 mg.

In some embodiments, the checkpoint inhibitor is administered once every six weeks from about 1 mg to about 300 mg.

In some embodiments, the checkpoint inhibitor is administered once every three weeks from about 1 mg to about 200 mg.

In some embodiments, the checkpoint inhibitor is administered once every six weeks from about 1 mg to about 200 mg.

In some embodiments, the checkpoint inhibitor is administered once every three weeks from about 10 mg to about 500 mg.

In some embodiments, the checkpoint inhibitor is administered once every six weeks from about 10 mg to about 500 mg.

In some embodiments, the checkpoint inhibitor is administered once every three weeks from about 200 mg to about 500 mg.

In some embodiments, the checkpoint inhibitor is administered once every six weeks from about 200 mg to about 500 mg.

In some embodiments, the checkpoint inhibitor is administered once every three weeks at about 200 mg.

In some embodiments, the checkpoint inhibitor is administered once every six weeks at about 200 mg.

In some embodiments, the checkpoint inhibitor is administered once every three weeks at about 400 mg.

In some embodiments, the checkpoint inhibitor is administered once every six weeks at about 400 mg.

In some embodiments, pembrolizumab in the present application is formulated for intravenous administration at the various doses in the methods, compositions, medicaments, therapeutic combinations, combinational therapies, and uses, described herein.

In some embodiments, pembrolizumab is administered at an amount of about 1 mg to about 500 mg.

In some embodiments, pembrolizumab is administered at an amount of about 1 mg to about 400 mg.

In some embodiments, pembrolizumab is administered at an amount of about 1 mg to about 300 mg.

In some embodiments, pembrolizumab is administered at an amount of about 1 mg to about 200 mg.

In some embodiments, pembrolizumab is administered at an amount of about 100 mg to about 500 mg.

In some embodiments, pembrolizumab is administered at an amount of about 100 mg to about 450 mg.

In some embodiments, pembrolizumab is administered at an amount of about 100 mg to about 400 mg.

In some embodiments, pembrolizumab is administered at an amount of about 100 mg to about 350 mg.

In some embodiments, pembrolizumab is administered at an amount of about 100 mg to about 300 mg.

In some embodiments, pembrolizumab is administered at an amount of about 100 mg to about 290 mg.

In some embodiments, pembrolizumab is administered at an amount of about 100 mg to about 280 mg.

In some embodiments, pembrolizumab is administered at an amount of about 100 mg to about 270 mg.

In some embodiments, pembrolizumab is administered at an amount of about 100 mg to about 260 mg.

In some embodiments, pembrolizumab is administered at an amount of about 100 mg to about 250 mg.

In some embodiments, pembrolizumab is administered at an amount of about 100 mg to about 240 mg.

In some embodiments, pembrolizumab is administered at an amount of about 100 mg to about 230 mg.

In some embodiments, pembrolizumab is administered at an amount of about 100 mg to about 220 mg.

In some embodiments, pembrolizumab is administered at an amount of about 100 mg to about 210 mg.

In some embodiments, pembrolizumab is administered at an amount of about 100 mg to about 200 mg.

In some embodiments, pembrolizumab is administered at an amount of about 100 mg to about 190 mg.

In some embodiments, pembrolizumab is administered at an amount of about 100 mg to about 180 mg.

In some embodiments, pembrolizumab is administered at an amount of about 100 mg to about 170 mg.

In some embodiments, pembrolizumab is administered at an amount of about 100 mg to about 160 mg.

In some embodiments, pembrolizumab is administered at an amount of about 100 mg to about 150 mg.

In some embodiments, pembrolizumab is administered at an amount of about 100 mg to about 140 mg.

In some embodiments, pembrolizumab is administered at an amount of about 100 mg to about 130 mg.

In some embodiments, pembrolizumab is administered at an amount of about 100 mg to about 120 mg.

In some embodiments, pembrolizumab is administered at an amount of about 100 mg to about 110 mg.

In some embodiments, pembrolizumab is administered at an amount of about 150 mg to about 500 mg.

In some embodiments, pembrolizumab is administered at an amount of about 150 mg to about 450 mg.

In some embodiments, pembrolizumab is administered at an amount of about 150 mg to about 400 mg.

In some embodiments, pembrolizumab is administered at an amount of about 150 mg to about 350 mg.

In some embodiments, pembrolizumab is administered at an amount of about 150 mg to about 300 mg.

In some embodiments, pembrolizumab is administered at an amount of about 150 mg to about 290 mg.

In some embodiments, pembrolizumab is administered at an amount of about 150 mg to about 280 mg.

In some embodiments, pembrolizumab is administered at an amount of about 150 mg to about 270 mg.

In some embodiments, pembrolizumab is administered at an amount of about 150 mg to about 260 mg.

In some embodiments, pembrolizumab is administered at an amount of about 150 mg to about 250 mg.

In some embodiments, pembrolizumab is administered at an amount of about 150 mg to about 240 mg.

In some embodiments, pembrolizumab is administered at an amount of about 150 mg to about 230 mg.

In some embodiments, pembrolizumab is administered at an amount of about 150 mg to about 220 mg.

In some embodiments, pembrolizumab is administered at an amount of about 150 mg to about 210 mg.

In some embodiments, pembrolizumab is administered at an amount of about 150 mg to about 200 mg.

In some embodiments, pembrolizumab is administered at an amount of about 150 mg to about 190 mg.

In some embodiments, pembrolizumab is administered at an amount of about 150 mg to about 180 mg.

In some embodiments, pembrolizumab is administered at an amount of about 150 mg to about 170 mg.

In some embodiments, pembrolizumab is administered at an amount of about 150 mg to about 160 mg.

In some embodiments, pembrolizumab is administered at an amount of about 200 mg to about 500 mg.

In some embodiments, pembrolizumab is administered at an amount of about 200 mg to about 450 mg.

In some embodiments, pembrolizumab is administered at an amount of about 200 mg to about 400 mg.

In some embodiments, pembrolizumab is administered at an amount of about 200 mg to about 350 mg.

In some embodiments, pembrolizumab is administered at an amount of about 200 mg to about 300 mg.

In some embodiments, pembrolizumab is administered at an amount of about 200 mg to about 290 mg.

In some embodiments, pembrolizumab is administered at an amount of about 200 mg to about 280 mg.

In some embodiments, pembrolizumab is administered at an amount of about 200 mg to about 270 mg.

In some embodiments, pembrolizumab is administered at an amount of about 200 mg to about 260 mg.

In some embodiments, pembrolizumab is administered at an amount of about 200 mg to about 250 mg.

In some embodiments, pembrolizumab is administered at an amount of about 200 mg to about 240 mg.

In some embodiments, pembrolizumab is administered at an amount of about 200 mg to about 230 mg.

In some embodiments, pembrolizumab is administered at an amount of about 200 mg to about 220 mg.

In some embodiments, pembrolizumab is administered at an amount of about 200 mg to about 210 mg.

In some embodiments, pembrolizumab is administered at about 500 mg.

In some embodiments, pembrolizumab is administered at about 450 mg.

In some embodiments, pembrolizumab is administered at about 440 mg.

In some embodiments, pembrolizumab is administered at about 430 mg.

In some embodiments, pembrolizumab is administered at about 420 mg.

In some embodiments, pembrolizumab is administered at about 410 mg.

In some embodiments, pembrolizumab is administered at about 400 mg.

In some embodiments, pembrolizumab is administered at about 390 mg.

In some embodiments, pembrolizumab is administered at about 380 mg.

In some embodiments, pembrolizumab is administered at about 370 mg.

In some embodiments, pembrolizumab is administered at about 360 mg.

In some embodiments, pembrolizumab is administered at about 350 mg.

In some embodiments, pembrolizumab is administered at about 340 mg.

In some embodiments, pembrolizumab is administered at about 330 mg.

In some embodiments, pembrolizumab is administered at about 320 mg In some embodiments, pembrolizumab is administered at about 310 mg.

In some embodiments, pembrolizumab is administered at about 300 mg.

In some embodiments, pembrolizumab is administered at about 290 mg.

In some embodiments, pembrolizumab is administered at about 280 mg.

In some embodiments, pembrolizumab is administered at about 270 mg.

In some embodiments, pembrolizumab is administered at about 260 mg.

In some embodiments, pembrolizumab is administered at about 250 mg.

In some embodiments, pembrolizumab is administered at about 240 mg.

In some embodiments, pembrolizumab is administered at about 230 mg.

In some embodiments, pembrolizumab is administered at about 220 mg In some embodiments, pembrolizumab is administered at about 210 mg.

In some embodiments, pembrolizumab is administered at about 200 mg.

In some embodiments, pembrolizumab is administered at about 190 mg.

In some embodiments, pembrolizumab is administered at about 180 mg.

In some embodiments, pembrolizumab is administered at about 170 mg.

In some embodiments, pembrolizumab is administered at about 160 mg.

In some embodiments, pembrolizumab is administered at about 150 mg.

In some embodiments, pembrolizumab is administered at about 140 mg.

In some embodiments, pembrolizumab is administered at about 130 mg.

In some embodiments, pembrolizumab is administered at about 120 mg In some embodiments, pembrolizumab is administered at about 110 mg.

In some embodiments, pembrolizumab is administered at about 100 mg.

In some embodiments, pembrolizumab is administered at about 50 mg.

In some embodiments, pembrolizumab in the present application is formulated for intravenous administration at the various doses described herein with the various dosing frequencies in the methods, compositions, medicaments, therapeutic combinations, combinational therapies, and uses, described herein.

In some embodiments, pembrolizumab is administered on consecutive days.

In some embodiments, pembrolizumab is administered 1-7 times per week.

In some embodiments, pembrolizumab is administered 2-7 times per week.

In some embodiments, pembrolizumab is administered 3-7 times per week.

In some embodiments, pembrolizumab is administered 4-7 times per week.

In some embodiments, pembrolizumab is administered 5-7 times per week.

In some embodiments, pembrolizumab is administered 6-7 times per week.

In some embodiments, pembrolizumab is administered 1-6 times per week. In some embodiments, pembrolizumab is administered 1-5 times per week. In some embodiments, pembrolizumab is administered 1-4 times per week. In some embodiments, pembrolizumab is administered 1-3 times per week. In some embodiments, pembrolizumab is administered 1-2 times per week.

In some embodiments, pembrolizumab is administered 2-6 times per week. In some embodiments, pembrolizumab is administered 2-5 times per week. In some embodiments, pembrolizumab is administered 2-4 times per week. In some embodiments, pembrolizumab is administered 2-3 times per week.

In some embodiments, pembrolizumab is administered 3-6 times per week. In some embodiments, pembrolizumab is administered 3-5 times per week. In some embodiments, pembrolizumab is administered 3-4 times per week.

In some embodiments, pembrolizumab is administered 4-6 times per week. In some embodiments, pembrolizumab is administered 4-5 times per week.

In some embodiments, pembrolizumab is administered 5-6 times per week.

In some embodiments, pembrolizumab is administered at least 1 time per week.

In some embodiments, pembrolizumab is administered at least 2 times per week.

In some embodiments, pembrolizumab is administered at least 3 times per week.

In some embodiments, pembrolizumab is administered at least 4 times per week.

In some embodiments, pembrolizumab is administered at least 5 times per week.

In some embodiments, pembrolizumab is administered at least 6 times per week.

In some embodiments, pembrolizumab is administered daily per week.

In some embodiments, pembrolizumab is administered at least 1 time per week at an amount of about 1 mg to about 500 mg.

In some embodiments, pembrolizumab is administered at least 1 time per week at an amount of about 1 mg to about 400 mg.

In some embodiments, pembrolizumab is administered at least 1 time per week at an amount of about 1 mg to about 300 mg.

In some embodiments, pembrolizumab is administered at least 1 time per week at an amount of about 1 mg to about 200 mg.

In some embodiments, pembrolizumab is administered at least 1 time per week at an amount of about 10 mg to about 500 mg.

In some embodiments, pembrolizumab is administered at least 1 time per week at an amount of about 200 mg to about 500 mg.

In some embodiments, pembrolizumab is administered at least 1 time per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 1 time per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 1 time per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg.

In some embodiments, pembrolizumab is administered at least 1 time per week at an amount of about 400 mg. In some embodiments, pembrolizumab is administered at least 1 time per week at an amount of about 200 mg.

In some embodiments, pembrolizumab is administered at least 2 times per week at an amount of about 1 mg to about 500 mg.

In some embodiments, pembrolizumab is administered at least 2 times per week at an amount of about 1 mg to about 400 mg.

In some embodiments, pembrolizumab is administered at least 2 times per week at an amount of about 1 mg to about 300 mg.

In some embodiments, pembrolizumab is administered at least 2 times per week at an amount of about 1 mg to about 200 mg.

In some embodiments, pembrolizumab is administered at least 2 times per week at an amount of about 10 mg to about 500 mg.

In some embodiments, pembrolizumab is administered at least 2 times per week at an amount of about 200 mg to about 500 mg.

In some embodiments, pembrolizumab is administered at least 2 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 2 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 2 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, pembrolizumab is administered at least 2 times per week at an amount of about 400 mg. In some embodiments, pembrolizumab is administered at least 2 times per week at an amount of about 200 mg.

In some embodiments, pembrolizumab is administered at least 3 times per week at an amount of about 1 mg to about 500 mg.

In some embodiments, pembrolizumab is administered at least 3 times per week at an amount of about 1 mg to about 400 mg.

In some embodiments, pembrolizumab is administered at least 3 times per week at an amount of about 1 mg to about 300 mg.

In some embodiments, pembrolizumab is administered at least 3 times per week at an amount of about 1 mg to about 200 mg.

In some embodiments, pembrolizumab is administered at least 3 times per week at an amount of about 10 mg to about 500 mg.

In some embodiments, pembrolizumab is administered at least 3 times per week at an amount of about 200 mg to about 500 mg.

In some embodiments, pembrolizumab is administered at least 3 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 3 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 3 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, pembrolizumab is administered at least 3 times per week at an amount of about 400 mg. In some embodiments, pembrolizumab is administered at least 3 times per week at an amount of about 200 mg.

In some embodiments, pembrolizumab is administered at least 4 times per week at an amount of about 1 mg to about 500 mg.

In some embodiments, pembrolizumab is administered at least 4 times per week at an amount of about 1 mg to about 400 mg.

In some embodiments, pembrolizumab is administered at least 4 times per week at an amount of about 1 mg to about 300 mg.

In some embodiments, pembrolizumab is administered at least 4 times per week at an amount of about 1 mg to about 200 mg.

In some embodiments, pembrolizumab is administered at least 4 times per week at an amount of about 10 mg to about 500 mg.

In some embodiments, pembrolizumab is administered at least 4 times per week at an amount of about 200 mg to about 500 mg.

In some embodiments, pembrolizumab is administered at least 4 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 4 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 4 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, pembrolizumab is administered at least 4 times per week at an amount of about 400 mg. In some embodiments, pembrolizumab is administered at least 4 times per week at an amount of about 200 mg.

In some embodiments, pembrolizumab is administered at least 5 times per week at an amount of about 1 mg to about 500 mg.

In some embodiments, pembrolizumab is administered at least 5 times per week at an amount of about 1 mg to about 400 mg.

In some embodiments, pembrolizumab is administered at least 5 times per week at an amount of about 1 mg to about 300 mg.

In some embodiments, pembrolizumab is administered at least 5 times per week at an amount of about 1 mg to about 200 mg.

In some embodiments, pembrolizumab is administered at least 5 times per week at an amount of about 10 mg to about 500 mg.

In some embodiments, pembrolizumab is administered at least 5 times per week at an amount of about 200 mg to about 500 mg.

In some embodiments, pembrolizumab is administered at least 5 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 5 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 5 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, pembrolizumab is administered at least 5 times per week at an amount of about 400 mg. In some embodiments, pembrolizumab is administered at least 5 times per week at an amount of about 200 mg.

In some embodiments, pembrolizumab is administered at least 6 times per week at an amount of about 1 mg to about 500 mg.

In some embodiments, pembrolizumab is administered at least 6 times per week at an amount of about 1 mg to about 400 mg.

In some embodiments, pembrolizumab is administered at least 6 times per week at an amount of about 1 mg to about 300 mg.

In some embodiments, pembrolizumab is administered at least 6 times per week at an amount of about 1 mg to about 200 mg.

In some embodiments, pembrolizumab is administered at least 6 times per week at an amount of about 10 mg to about 500 mg.

In some embodiments, pembrolizumab is administered at least 6 times per week at an amount of about 200 mg to about 500 mg.

In some embodiments, pembrolizumab is administered at least 6 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg. In some embodiments, pembrolizumab is administered at least 6 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg. In some embodiments, pembrolizumab is administered at least 6 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg. In some embodiments, pembrolizumab is administered at least 6 times per week at an amount of about 400 mg. In some embodiments, pembrolizumab is administered at least 6 times per week at an amount of about 200 mg.

In some embodiments, pembrolizumab is administered once every three weeks.

In some embodiments, pembrolizumab is administered once every six weeks.

In some embodiments, pembrolizumab is administered once every three weeks from about 1 mg to about 500 mg.

In some embodiments, pembrolizumab is administered once every six weeks about 1 mg to about 500 mg.

In some embodiments, pembrolizumab is administered once every three weeks from about 1 mg to about 400 mg.

In some embodiments, pembrolizumab is administered once every six weeks from about 1 mg to about 400 mg.

In some embodiments, pembrolizumab is administered once every three weeks from about 1 mg to about 300 mg.

In some embodiments, pembrolizumab is administered once every six weeks from about 1 mg to about 300 mg.

In some embodiments, pembrolizumab is administered once every three weeks from about 1 mg to about 200 mg.

In some embodiments, pembrolizumab is administered once every six weeks from about 1 mg to about 200 mg.

In some embodiments, pembrolizumab is administered once every three weeks from about 10 mg to about 500 mg.

In some embodiments, pembrolizumab is administered once every six weeks from about 10 mg to about 500 mg.

In some embodiments, pembrolizumab is administered once every three weeks from about 200 mg to about 500 mg.

In some embodiments, pembrolizumab is administered once every six weeks from about 200 mg to about 500 mg.

In some embodiments, pembrolizumab is administered once every three weeks at about 200 mg.

In some embodiments, pembrolizumab is administered once every six weeks at about 200 mg.

In some embodiments, pembrolizumab is administered once every three weeks at about 400 mg.

In some embodiments, pembrolizumab is administered once every six weeks at about 400 mg.

### Paclitaxel, Compound A, and a Checkpoint Inhibitor

In some embodiments, any dose described herein for paclitaxel can be combined with any dose described herein for Compound A and/or the checkpoint inhibitor, which combination can be combined with any dosing frequency described herein for paclitaxel, Compound A, and a checkpoint inhibitor.

In some embodiments, paclitaxel is formulated for oral administration.

In some embodiments, Compound A is formulated for oral administration.

In some embodiments, the checkpoint inhibitor is formulated for intravenous administration.

In some embodiments, the checkpoint inhibitor is pembrolizumab.

In some embodiments, pembrolizumab is formulated for intravenous administration.

In some embodiments, the paclitaxel is administered at an amount of about 100 mg to about 400 mg in combination with Compound A and a checkpoint inhibitor.

In some embodiments, the paclitaxel is administered at an amount of about 100 mg to about 350 mg in combination with Compound A and a checkpoint inhibitor.

In some embodiments, the paclitaxel is administered at an amount of about 100 mg to about 300 mg in combination with Compound A and a checkpoint inhibitor.

In some embodiments, the paclitaxel is administered at an amount of about 150 mg to about 400 mg in combination with Compound A and a checkpoint inhibitor.

In some embodiments, the paclitaxel is administered at an amount of about 150 mg to about 350 mg in combination with Compound A and a checkpoint inhibitor.

In some embodiments, the paclitaxel is administered at an amount of about 150 mg to about 340 mg in combination with Compound A and a checkpoint inhibitor.

In some embodiments, the paclitaxel is administered at an amount of about 150 mg to about 330 mg in combination with Compound A and a checkpoint inhibitor.

In some embodiments, the paclitaxel is administered at an amount of about 150 mg to about 320 mg in combination with Compound A and a checkpoint inhibitor.

In some embodiments, the paclitaxel is administered at an amount of about 150 mg to about 310 mg in combination with Compound A and a checkpoint inhibitor.

In some embodiments, the paclitaxel is administered at an amount of about 150 mg to about 300 mg in combination with Compound A and a checkpoint inhibitor.

In some embodiments, the paclitaxel is administered at an amount of about 150 mg to about 290 mg in combination with Compound A and a checkpoint inhibitor.

In some embodiments, the paclitaxel is administered at an amount of about 150 mg to about 280 mg in combination with Compound A and a checkpoint inhibitor.

In some embodiments, the paclitaxel is administered at an amount of about 150 mg to about 270 mg in combination with Compound A and a checkpoint inhibitor.

In some embodiments, the paclitaxel is administered at an amount of about 150 mg to about 260 mg in combination with Compound A and a checkpoint inhibitor.

In some embodiments, the paclitaxel is administered at an amount of about 150 mg to about 250 mg in combination with Compound A and a checkpoint inhibitor.

In some embodiments, the paclitaxel is administered at an amount of about 200 mg to about 400 mg in combination with Compound A and a checkpoint inhibitor.

In some embodiments, the paclitaxel is administered at an amount of about 200 mg to about 350 mg in combination with Compound A and a checkpoint inhibitor.

In some embodiments, the paclitaxel is administered at an amount of about 200 mg to about 340 mg in combination with Compound A and a checkpoint inhibitor.

In some embodiments, the paclitaxel is administered at an amount of about 200 mg to about 330 mg in combination with Compound A and a checkpoint inhibitor.

In some embodiments, the paclitaxel is administered at an amount of about 200 mg to about 320 mg in combination with Compound A and a checkpoint inhibitor.

In some embodiments, the paclitaxel is administered at an amount of about 200 mg to about 310 mg in combination with Compound A and a checkpoint inhibitor.

In some embodiments, the paclitaxel is administered at an amount of about 200 mg to about 300 mg in combination with Compound A and a checkpoint inhibitor.

In some embodiments, the paclitaxel is administered at an amount of about 200 mg to about 295 mg in combination with Compound A and a checkpoint inhibitor.

In some embodiments, the paclitaxel is administered at an amount of about 200 mg to about 290 mg in combination with Compound A and a checkpoint inhibitor.

In some embodiments, the paclitaxel is administered at an amount of about 200 mg to about 285 mg in combination with Compound A and a checkpoint inhibitor.

In some embodiments, the paclitaxel is administered at an amount of about 200 mg to about 280 mg in combination with Compound A and a checkpoint inhibitor.

In some embodiments, the paclitaxel is administered at an amount of about 200 mg to about 275 mg in combination with Compound A and a checkpoint inhibitor.

In some embodiments, the paclitaxel is administered at an amount of about 200 mg to about 270 mg in combination with Compound A and a checkpoint inhibitor.

In some embodiments, the paclitaxel is administered at an amount of about 200 mg to about 265 mg in combination with Compound A and a checkpoint inhibitor.

In some embodiments, the paclitaxel is administered at an amount of about 200 mg to about 260 mg in combination with Compound A and a checkpoint inhibitor.

In some embodiments, the paclitaxel is administered at an amount of about 200 mg to about 255 mg in combination with Compound A and a checkpoint inhibitor.

In some embodiments, the paclitaxel is administered at an amount of about 200 mg to about 250 mg in combination with Compound A and a checkpoint inhibitor.

In some embodiments, the paclitaxel is administered at an amount of about 250 mg to about 400 mg in combination with Compound A and a checkpoint inhibitor.

In some embodiments, the paclitaxel is administered at an amount of about 250 mg to about 350 mg in combination with Compound A and a checkpoint inhibitor.

In some embodiments, the paclitaxel is administered at an amount of about 250 mg to about 340 mg in combination with Compound A and a checkpoint inhibitor.

In some embodiments, the paclitaxel is administered at an amount of about 250 mg to about 330 mg in combination with Compound A and a checkpoint inhibitor.

In some embodiments, the paclitaxel is administered at an amount of about 250 mg to about 320 mg in combination with Compound A and a checkpoint inhibitor.

In some embodiments, the paclitaxel is administered at an amount of about 250 mg to about 310 mg in combination with Compound A and a checkpoint inhibitor.

In some embodiments, the paclitaxel is administered at an amount of about 250 mg to about 300 mg in combination with Compound A and a checkpoint inhibitor.

In some embodiments, the paclitaxel is administered at an amount of about 250 mg to about 290 mg in combination with Compound A and a checkpoint inhibitor.

In some embodiments, the paclitaxel is administered at an amount of about 250 mg to about 280 mg in combination with Compound A and a checkpoint inhibitor.

In some embodiments, the paclitaxel is administered at an amount of about 250 mg to about 270 mg in combination with Compound A and a checkpoint inhibitor.

In some embodiments, the paclitaxel is administered at an amount of about 250 mg to about 260 mg in combination with Compound A and a checkpoint inhibitor.

In some embodiments, the paclitaxel is administered at an amount of about 100 mg in combination with Compound A and a checkpoint inhibitor.

In some embodiments, the paclitaxel is administered at an amount of about 105 mg in combination with Compound A and a checkpoint inhibitor.

In some embodiments, the paclitaxel is administered at an amount of about 110 mg in combination with Compound A and a checkpoint inhibitor.

In some embodiments, the paclitaxel is administered at an amount of about 115 mg in combination with Compound A and a checkpoint inhibitor.

In some embodiments, the paclitaxel is administered at an amount of about 120 mg in combination with Compound A and a checkpoint inhibitor.

In some embodiments, the paclitaxel is administered at an amount of about 125 mg in combination with Compound A and a checkpoint inhibitor.

In some embodiments, the paclitaxel is administered at an amount of about 130 mg in combination with Compound A and a checkpoint inhibitor.

In some embodiments, the paclitaxel is administered at an amount of about 135 mg in combination with Compound A and a checkpoint inhibitor.

In some embodiments, the paclitaxel is administered at an amount of about 140 mg in combination with Compound A and a checkpoint inhibitor.

In some embodiments, the paclitaxel is administered at an amount of about 145 mg in combination with Compound A and a checkpoint inhibitor.

In some embodiments, the paclitaxel is administered at an amount of about 150 mg in combination with Compound A and a checkpoint inhibitor.

In some embodiments, the paclitaxel is administered at an amount of about 155 mg in combination with Compound A and a checkpoint inhibitor.

In some embodiments, the paclitaxel is administered at an amount of about 160 mg in combination with Compound A and a checkpoint inhibitor.

In some embodiments, the paclitaxel is administered at an amount of about 165 mg in combination with Compound A and a checkpoint inhibitor.

In some embodiments, the paclitaxel is administered at an amount of about 170 mg in combination with Compound A and a checkpoint inhibitor.

In some embodiments, the paclitaxel is administered at an amount of about 175 mg in combination with Compound A and a checkpoint inhibitor.

In some embodiments, the paclitaxel is administered at an amount of about 180 mg in combination with Compound A and a checkpoint inhibitor.

In some embodiments, the paclitaxel is administered at an amount of about 185 mg in combination with Compound A and a checkpoint inhibitor.

In some embodiments, the paclitaxel is administered at an amount of about 190 mg in combination with Compound A and a checkpoint inhibitor.

In some embodiments, the paclitaxel is administered at an amount of about 195 mg in combination with Compound A and a checkpoint inhibitor.

In some embodiments, the paclitaxel is administered at an amount of about 200 mg in combination with Compound A and a checkpoint inhibitor.

In some embodiments, the paclitaxel is administered at an amount of about 205 mg in combination with Compound A and a checkpoint inhibitor.

In some embodiments, the paclitaxel is administered at an amount of about 210 mg in combination with Compound A and a checkpoint inhibitor.

In some embodiments, the paclitaxel is administered at an amount of about 215 mg in combination with Compound A and a checkpoint inhibitor.

In some embodiments, the paclitaxel is administered at an amount of about 220 mg in combination with Compound A and a checkpoint inhibitor.

In some embodiments, the paclitaxel is administered at an amount of about 225 mg in combination with Compound A and a checkpoint inhibitor.

In some embodiments, the paclitaxel is administered at an amount of about 230 mg in combination with Compound A and a checkpoint inhibitor.

In some embodiments, the paclitaxel is administered at an amount of about 235 mg in combination with Compound A and a checkpoint inhibitor.

In some embodiments, the paclitaxel is administered at an amount of about 240 mg in combination with Compound A and a checkpoint inhibitor.

In some embodiments, the paclitaxel is administered at an amount of about 245 mg in combination with Compound A and a checkpoint inhibitor.

In some embodiments, the paclitaxel is administered at an amount of about 250 mg in combination with Compound A and a checkpoint inhibitor.

In some embodiments, the paclitaxel is administered at an amount of about 255 mg in combination with Compound A and a checkpoint inhibitor.

In some embodiments, the paclitaxel is administered at an amount of about 260 mg in combination with Compound A and a checkpoint inhibitor.

In some embodiments, the paclitaxel is administered at an amount of about 265 mg in combination with Compound A and a checkpoint inhibitor.

In some embodiments, the paclitaxel is administered at an amount of about 270 mg in combination with Compound A and a checkpoint inhibitor.

In some embodiments, the paclitaxel is administered at an amount of about 275 mg in combination with Compound A and a checkpoint inhibitor.

In some embodiments, the paclitaxel is administered at an amount of about 280 mg in combination with Compound A and a checkpoint inhibitor.

In some embodiments, the paclitaxel is administered at an amount of about 285 mg in combination with Compound A and a checkpoint inhibitor.

In some embodiments, the paclitaxel is administered at an amount of about 290 mg in combination with Compound A and a checkpoint inhibitor.

In some embodiments, the paclitaxel is administered at an amount of about 295 mg in combination with Compound A and a checkpoint inhibitor.

In some embodiments, the paclitaxel is administered at an amount of about 300 mg in combination with Compound A and a checkpoint inhibitor.

In some embodiments, the paclitaxel is administered at an amount of about 305 mg in combination with Compound A and a checkpoint inhibitor.

In some embodiments, the paclitaxel is administered at an amount of about 310 mg in combination with Compound A and a checkpoint inhibitor.

In some embodiments, the paclitaxel is administered at an amount of about 315 mg in combination with Compound A and a checkpoint inhibitor.

In some embodiments, the paclitaxel is administered at an amount of about 320 mg in combination with Compound A and a checkpoint inhibitor.

In some embodiments, the paclitaxel is administered at an amount of about 325 mg in combination with Compound A and a checkpoint inhibitor.

In some embodiments, the paclitaxel is administered at an amount of about 330 mg in combination with Compound A and a checkpoint inhibitor.

In some embodiments, the paclitaxel is administered at an amount of about 335 mg in combination with Compound A and a checkpoint inhibitor.

In some embodiments, the paclitaxel is administered at an amount of about 340 mg in combination with Compound A and a checkpoint inhibitor.

In some embodiments, the paclitaxel is administered at an amount of about 345 mg in combination with Compound A and a checkpoint inhibitor.

In some embodiments, the paclitaxel is administered at an amount of about 350 mg in combination with Compound A and a checkpoint inhibitor.

In some embodiments, the paclitaxel is administered at an amount of about 355 mg in combination with Compound A and a checkpoint inhibitor.

In some embodiments, the paclitaxel is administered at an amount of about 360 mg in combination with Compound A and a checkpoint inhibitor.

In some embodiments, the paclitaxel is administered at an amount of about 365 mg in combination with Compound A and a checkpoint inhibitor.

In some embodiments, the paclitaxel is administered at an amount of about 370 mg in combination with Compound A and a checkpoint inhibitor.

In some embodiments, the paclitaxel is administered at an amount of about 375 mg in combination with Compound A and a checkpoint inhibitor.

In some embodiments, the paclitaxel is administered at an amount of about 380 mg in combination with Compound A and a checkpoint inhibitor.

In some embodiments, the paclitaxel is administered at an amount of about 385 mg in combination with Compound A and a checkpoint inhibitor.

In some embodiments, the paclitaxel is administered at an amount of about 390 mg in combination with Compound A and a checkpoint inhibitor.

In some embodiments, the paclitaxel is administered at an amount of about 395 mg in combination with Compound A and a checkpoint inhibitor.

In some embodiments, the paclitaxel is administered at an amount of about 400 mg in combination with Compound A and a checkpoint inhibitor.

In some embodiments, the paclitaxel is administered in combination with Compound A and a checkpoint inhibitor on consecutive days.

In some embodiments, the paclitaxel is administered in combination with Compound A and a checkpoint inhibitor 1-7 times per week.

In some embodiments, the paclitaxel is administered in combination with Compound A and a checkpoint inhibitor 2-7 times per week.

In some embodiments, the paclitaxel is administered in combination with Compound A and a checkpoint inhibitor 3-7 times per week.

In some embodiments, the paclitaxel is administered in combination with Compound A and a checkpoint inhibitor 4-7 times per week.

In some embodiments, the paclitaxel is administered in combination with Compound A and a checkpoint inhibitor 5-7 times per week.

In some embodiments, the paclitaxel is administered in combination with Compound A and a checkpoint inhibitor 6-7 times per week.

In some embodiments, the paclitaxel is administered in combination with Compound A and a checkpoint inhibitor 1-6 times per week. In some embodiments, the paclitaxel is administered in combination with Compound A and a checkpoint inhibitor 1-5 times per week.

In some embodiments, the paclitaxel is administered in combination with Compound A and a checkpoint inhibitor 1-4 times per week. In some embodiments, the paclitaxel is administered in combination with Compound A and a checkpoint inhibitor 1-3 times per week. In some embodiments, the paclitaxel is administered in combination with Compound A and a checkpoint inhibitor 1-2 times per week.

In some embodiments, the paclitaxel is administered in combination with Compound A and a checkpoint inhibitor 2-6 times per week. In some embodiments, the paclitaxel is administered in combination with Compound A and a checkpoint inhibitor 2-5 times per week.

In some embodiments, the paclitaxel is administered in combination with Compound A and a checkpoint inhibitor 2-4 times per week. In some embodiments, the paclitaxel is administered in combination with Compound A and a checkpoint inhibitor 2-3 times per week.

In some embodiments, the paclitaxel is administered in combination with Compound A and a checkpoint inhibitor 3-6 times per week. In some embodiments, the paclitaxel is administered in combination with Compound A and a checkpoint inhibitor 3-5 times per week.

In some embodiments, the paclitaxel is administered in combination with Compound A and a checkpoint inhibitor 3-4 times per week.

In some embodiments, the paclitaxel is administered in combination with Compound A and a checkpoint inhibitor 4-6 times per week. In some embodiments, the paclitaxel is administered in combination with Compound A and a checkpoint inhibitor 4-5 times per week.

In some embodiments, the paclitaxel is administered in combination with Compound A and a checkpoint inhibitor 5-6 times per week.

In some embodiments, the paclitaxel is administered in combination with Compound A and a checkpoint inhibitor at least 1 time per week.

In some embodiments, the paclitaxel is administered in combination with Compound A and a checkpoint inhibitor at least 2 times per week.

In some embodiments, the paclitaxel is administered in combination with Compound A and a checkpoint inhibitor at least 3 times per week.

In some embodiments, the paclitaxel is administered in combination with Compound A and a checkpoint inhibitor at least 4 times per week.

In some embodiments, the paclitaxel is administered in combination with Compound A and a checkpoint inhibitor at least 5 times per week.

In some embodiments, the paclitaxel is administered in combination with Compound A and a checkpoint inhibitor at least 6 times per week.

In some embodiments, the paclitaxel is administered in combination with Compound A and a checkpoint inhibitor daily per week.

In some embodiments, the paclitaxel is administered in combination with Compound A and a checkpoint inhibitor at least 1 time a week.

In some embodiments, the paclitaxel is administered at an amount of about 100 mg to about 400 mg in combination with Compound A and a checkpoint inhibitor at least 1 time a week.

In some embodiments, the paclitaxel is administered at an amount of about 100 mg to about 350 mg in combination with Compound A and a checkpoint inhibitor at least 1 time a week.

In some embodiments, the paclitaxel is administered at an amount of about 100 mg to about 300 mg in combination with Compound A and a checkpoint inhibitor at least 1 time a week.

In some embodiments, the paclitaxel is administered at an amount of about 150 mg to about 400 mg in combination with Compound A and a checkpoint inhibitor at least 1 time a week.

In some embodiments, the paclitaxel is administered at an amount of about 150 mg to about 350 mg in combination with Compound A and a checkpoint inhibitor at least 1 time a week.

In some embodiments, the paclitaxel is administered at an amount of about 150 mg to about 340 mg in combination with Compound A and a checkpoint inhibitor at least 1 time a week.

In some embodiments, the paclitaxel is administered at an amount of about 150 mg to about 330 mg in combination with Compound A and a checkpoint inhibitor at least 1 time a week.

In some embodiments, the paclitaxel is administered at an amount of about 150 mg to about 320 mg in combination with Compound A and a checkpoint inhibitor at least 1 time a week.

In some embodiments, the paclitaxel is administered at an amount of about 150 mg to about 310 mg in combination with Compound A and a checkpoint inhibitor at least 1 time a week.

In some embodiments, the paclitaxel is administered at an amount of about 150 mg to about 300 mg in combination with Compound A and a checkpoint inhibitor at least 1 time a week.

In some embodiments, the paclitaxel is administered at an amount of about 150 mg to about 290 mg in combination with Compound A and a checkpoint inhibitor at least 1 time a week.

In some embodiments, the paclitaxel is administered at an amount of about 150 mg to about 280 mg in combination with Compound A and a checkpoint inhibitor at least 1 time a week.

In some embodiments, the paclitaxel is administered at an amount of about 150 mg to about 270 mg in combination with Compound A and a checkpoint inhibitor at least 1 time a week.

In some embodiments, the paclitaxel is administered at an amount of about 150 mg to about 260 mg in combination with Compound A and a checkpoint inhibitor at least 1 time a week.

In some embodiments, the paclitaxel is administered at an amount of about 150 mg to about 250 mg in combination with Compound A and a checkpoint inhibitor at least 1 time a week.

In some embodiments, the paclitaxel is administered at an amount of about 200 mg to about 400 mg in combination with Compound A and a checkpoint inhibitor at least 1 time a week.

In some embodiments, the paclitaxel is administered at an amount of about 200 mg to about 350 mg in combination with Compound A and a checkpoint inhibitor at least 1 time a week.

In some embodiments, the paclitaxel is administered at an amount of about 200 mg to about 340 mg in combination with Compound A and a checkpoint inhibitor at least 1 time a week.

In some embodiments, the paclitaxel is administered at an amount of about 200 mg to about 330 mg in combination with Compound A and a checkpoint inhibitor at least 1 time a

In some embodiments, the paclitaxel is administered at an amount of about 200 mg to about 320 mg in combination with Compound A and a checkpoint inhibitor at least 1 time a week.

In some embodiments, the paclitaxel is administered at an amount of about 200 mg to about 310 mg in combination with Compound A and a checkpoint inhibitor at least 1 time a week.

In some embodiments, the paclitaxel is administered at an amount of about 200 mg to about 300 mg in combination with Compound A and a checkpoint inhibitor at least 1 time a week.

In some embodiments, the paclitaxel is administered at an amount of about 200 mg to about 295 mg in combination with Compound A and a checkpoint inhibitor at least 1 time a week.

In some embodiments, the paclitaxel is administered at an amount of about 200 mg to about 290 mg in combination with Compound A and a checkpoint inhibitor at least 1 time a week.

In some embodiments, the paclitaxel is administered at an amount of about 200 mg to about 285 mg in combination with Compound A and a checkpoint inhibitor at least 1 time a week.

In some embodiments, the paclitaxel is administered at an amount of about 200 mg to about 280 mg in combination with Compound A and a checkpoint inhibitor at least 1 time a week.

In some embodiments, the paclitaxel is administered at an amount of about 200 mg to about 275 mg in combination with Compound A and a checkpoint inhibitor at least 1 time a week.

In some embodiments, the paclitaxel is administered at an amount of about 200 mg to about 270 mg in combination with Compound A and a checkpoint inhibitor at least 1 time a week.

In some embodiments, the paclitaxel is administered at an amount of about 200 mg to about 265 mg in combination with Compound A and a checkpoint inhibitor at least 1 time a

In some embodiments, the paclitaxel is administered at an amount of about 200 mg to about 260 mg in combination with Compound A and a checkpoint inhibitor at least 1 time a week.

In some embodiments, the paclitaxel is administered at an amount of about 200 mg to about 255 mg in combination with Compound A and a checkpoint inhibitor at least 1 time a week.

In some embodiments, the paclitaxel is administered at an amount of about 200 mg to about 250 mg in combination with Compound A and a checkpoint inhibitor at least 1 time a week.

In some embodiments, the paclitaxel is administered at an amount of about 250 mg to about 400 mg in combination with Compound A and a checkpoint inhibitor at least 1 time a week.

In some embodiments, the paclitaxel is administered at an amount of about 250 mg to about 350 mg in combination with Compound A and a checkpoint inhibitor at least 1 time a week.

In some embodiments, the paclitaxel is administered at an amount of about 250 mg to about 340 mg in combination with Compound A and a checkpoint inhibitor at least 1 time a week.

In some embodiments, the paclitaxel is administered at an amount of about 250 mg to about 330 mg in combination with Compound A and a checkpoint inhibitor at least 1 time a week.

In some embodiments, the paclitaxel is administered at an amount of about 250 mg to about 320 mg in combination with Compound A and a checkpoint inhibitor at least 1 time a week.

In some embodiments, the paclitaxel is administered at an amount of about 250 mg to about 310 mg in combination with Compound A and a checkpoint inhibitor at least 1 time a week.

In some embodiments, the paclitaxel is administered at an amount of about 250 mg to about 300 mg in combination with Compound A and a checkpoint inhibitor at least 1 time a week.

In some embodiments, the paclitaxel is administered at an amount of about 250 mg to about 290 mg in combination with Compound A and a checkpoint inhibitor at least 1 time a week.

In some embodiments, the paclitaxel is administered at an amount of about 250 mg to about 280 mg in combination with Compound A and a checkpoint inhibitor at least 1 time a week.

In some embodiments, the paclitaxel is administered at an amount of about 250 mg to about 270 mg in combination with Compound A and a checkpoint inhibitor at least 1 time a week.

In some embodiments, the paclitaxel is administered at an amount of about 250 mg to about 260 mg in combination with Compound A and a checkpoint inhibitor at least 1 time a week.

In some embodiments, the paclitaxel is administered at an amount of about 100 mg in combination with Compound A and a checkpoint inhibitor at least 1 time a week.

In some embodiments, the paclitaxel is administered at an amount of about 105 mg in combination with Compound A and a checkpoint inhibitor at least 1 time a week.

In some embodiments, the paclitaxel is administered at an amount of about 110 mg in combination with Compound A and a checkpoint inhibitor at least 1 time a week.

In some embodiments, the paclitaxel is administered at an amount of about 115 mg in combination with Compound A and a checkpoint inhibitor at least 1 time a week.

In some embodiments, the paclitaxel is administered at an amount of about 120 mg in combination with Compound A and a checkpoint inhibitor at least 1 time a week.

In some embodiments, the paclitaxel is administered at an amount of about 125 mg in combination with Compound A and a checkpoint inhibitor at least 1 time a week.

In some embodiments, the paclitaxel is administered at an amount of about 130 mg in combination with Compound A and a checkpoint inhibitor at least 1 time a week.

In some embodiments, the paclitaxel is administered at an amount of about 135 mg in combination with Compound A and a checkpoint inhibitor at least 1 time a week.

In some embodiments, the paclitaxel is administered at an amount of about 140 mg in combination with Compound A and a checkpoint inhibitor at least 1 time a week.

In some embodiments, the paclitaxel is administered at an amount of about 145 mg in combination with Compound A and a checkpoint inhibitor at least 1 time a week.

In some embodiments, the paclitaxel is administered at an amount of about 150 mg in combination with Compound A and a checkpoint inhibitor at least 1 time a week.

In some embodiments, the paclitaxel is administered at an amount of about 155 mg in combination with Compound A and a checkpoint inhibitor at least 1 time a week.

In some embodiments, the paclitaxel is administered at an amount of about 160 mg in combination with Compound A and a checkpoint inhibitor at least 1 time a week.

In some embodiments, the paclitaxel is administered at an amount of about 165 mg in combination with Compound A and a checkpoint inhibitor at least 1 time a week.

In some embodiments, the paclitaxel is administered at an amount of about 170 mg in combination with Compound A and a checkpoint inhibitor at least 1 time a week.

In some embodiments, the paclitaxel is administered at an amount of about 175 mg in combination with Compound A and a checkpoint inhibitor at least 1 time a week.

In some embodiments, the paclitaxel is administered at an amount of about 180 mg in combination with Compound A and a checkpoint inhibitor at least 1 time a week.

In some embodiments, the paclitaxel is administered at an amount of about 185 mg in combination with Compound A and a checkpoint inhibitor at least 1 time a week.

In some embodiments, the paclitaxel is administered at an amount of about 190 mg in combination with Compound A and a checkpoint inhibitor at least 1 time a week.

In some embodiments, the paclitaxel is administered at an amount of about 195 mg in combination with Compound A and a checkpoint inhibitor at least 1 time a week.

In some embodiments, the paclitaxel is administered at an amount of about 200 mg in combination with Compound A and a checkpoint inhibitor at least 1 time a week.

In some embodiments, the paclitaxel is administered at an amount of about 205 mg in combination with Compound A and a checkpoint inhibitor at least 1 time a week.

In some embodiments, the paclitaxel is administered at an amount of about 210 mg in combination with Compound A and a checkpoint inhibitor at least 1 time a week.

In some embodiments, the paclitaxel is administered at an amount of about 215 mg in combination with Compound A and a checkpoint inhibitor at least 1 time a week.

In some embodiments, the paclitaxel is administered at an amount of about 220 mg in combination with Compound A and a checkpoint inhibitor at least 1 time a week.

In some embodiments, the paclitaxel is administered at an amount of about 225 mg in combination with Compound A and a checkpoint inhibitor at least 1 time a week.

In some embodiments, the paclitaxel is administered at an amount of about 230 mg in combination with Compound A and a checkpoint inhibitor at least 1 time a week.

In some embodiments, the paclitaxel is administered at an amount of about 235 mg in combination with Compound A and a checkpoint inhibitor at least 1 time a week.

In some embodiments, the paclitaxel is administered at an amount of about 240 mg in combination with Compound A and a checkpoint inhibitor at least 1 time a week.

In some embodiments, the paclitaxel is administered at an amount of about 245 mg in combination with Compound A and a checkpoint inhibitor at least 1 time a week.

In some embodiments, the paclitaxel is administered at an amount of about 250 mg in combination with Compound A and a checkpoint inhibitor at least 1 time a week.

In some embodiments, the paclitaxel is administered at an amount of about 255 mg in combination with Compound A and a checkpoint inhibitor at least 1 time a week.

In some embodiments, the paclitaxel is administered at an amount of about 260 mg in combination with Compound A and a checkpoint inhibitor at least 1 time a week.

In some embodiments, the paclitaxel is administered at an amount of about 265 mg in combination with Compound A and a checkpoint inhibitor at least 1 time a week.

In some embodiments, the paclitaxel is administered at an amount of about 270 mg in combination with Compound A and a checkpoint inhibitor at least 1 time a week.

In some embodiments, the paclitaxel is administered at an amount of about 275 mg in combination with Compound A and a checkpoint inhibitor at least 1 time a week.

In some embodiments, the paclitaxel is administered at an amount of about 280 mg in combination with Compound A and a checkpoint inhibitor at least 1 time a week.

In some embodiments, the paclitaxel is administered at an amount of about 285 mg in combination with Compound A and a checkpoint inhibitor at least 1 time a week.

In some embodiments, the paclitaxel is administered at an amount of about 290 mg in combination with Compound A and a checkpoint inhibitor at least 1 time a week.

In some embodiments, the paclitaxel is administered at an amount of about 295 mg in combination with Compound A and a checkpoint inhibitor at least 1 time a week.

In some embodiments, the paclitaxel is administered at an amount of about 300 mg in combination with Compound A and a checkpoint inhibitor at least 1 time a week.

In some embodiments, the paclitaxel is administered at an amount of about 305 mg in combination with Compound A and a checkpoint inhibitor at least 1 time a week.

In some embodiments, the paclitaxel is administered at an amount of about 310 mg in combination with Compound A and a checkpoint inhibitor at least 1 time a week.

In some embodiments, the paclitaxel is administered at an amount of about 315 mg in combination with Compound A and a checkpoint inhibitor at least 1 time a week.

In some embodiments, the paclitaxel is administered at an amount of about 320 mg in combination with Compound A and a checkpoint inhibitor at least 1 time a week.

In some embodiments, the paclitaxel is administered at an amount of about 325 mg in combination with Compound A and a checkpoint inhibitor at least 1 time a week.

In some embodiments, the paclitaxel is administered at an amount of about 330 mg in combination with Compound A and a checkpoint inhibitor at least 1 time a week.

In some embodiments, the paclitaxel is administered at an amount of about 335 mg in combination with Compound A and a checkpoint inhibitor at least 1 time a week.

In some embodiments, the paclitaxel is administered at an amount of about 340 mg in combination with Compound A and a checkpoint inhibitor at least 1 time a week.

In some embodiments, the paclitaxel is administered at an amount of about 345 mg in combination with Compound A and a checkpoint inhibitor at least 1 time a week.

In some embodiments, the paclitaxel is administered at an amount of about 350 mg in combination with Compound A and a checkpoint inhibitor at least 1 time a week.

In some embodiments, the paclitaxel is administered at an amount of about 355 mg in combination with Compound A and a checkpoint inhibitor at least 1 time a week.

In some embodiments, the paclitaxel is administered at an amount of about 360 mg in combination with Compound A and a checkpoint inhibitor at least 1 time a week.

In some embodiments, the paclitaxel is administered at an amount of about 365 mg in combination with Compound A and a checkpoint inhibitor at least 1 time a week.

In some embodiments, the paclitaxel is administered at an amount of about 370 mg in combination with Compound A and a checkpoint inhibitor at least 1 time a week.

In some embodiments, the paclitaxel is administered at an amount of about 375 mg in combination with Compound A and a checkpoint inhibitor at least 1 time a week.

In some embodiments, the paclitaxel is administered at an amount of about 380 mg in combination with Compound A and a checkpoint inhibitor at least 1 time a week.

In some embodiments, the paclitaxel is administered at an amount of about 385 mg in combination with Compound A and a checkpoint inhibitor at least 1 time a week.

In some embodiments, the paclitaxel is administered at an amount of about 390 mg in combination with Compound A and a checkpoint inhibitor at least 1 time a week.

In some embodiments, the paclitaxel is administered at an amount of about 395 mg in combination with Compound A and a checkpoint inhibitor at least 1 time a week.

In some embodiments, the paclitaxel is administered at an amount of about 400 mg in combination with Compound A and a checkpoint inhibitor at least 1 time a week.

In some embodiments, the paclitaxel is administered in combination with Compound A and a checkpoint inhibitor at least 2 times a week.

In some embodiments, the paclitaxel is administered at an amount of about 100 mg to about 400 mg in combination with Compound A and a checkpoint inhibitor at least 2 times a week.

In some embodiments, the paclitaxel is administered at an amount of about 100 mg to about 350 mg in combination with Compound A and a checkpoint inhibitor at least 2 times a week.

In some embodiments, the paclitaxel is administered at an amount of about 100 mg to about 300 mg in combination with Compound A and a checkpoint inhibitor at least 2 times a week.

In some embodiments, the paclitaxel is administered at an amount of about 150 mg to about 400 mg in combination with Compound A and a checkpoint inhibitor at least 2 times a week.

In some embodiments, the paclitaxel is administered at an amount of about 150 mg to about 350 mg in combination with Compound A and a checkpoint inhibitor at least 2 times a week.

In some embodiments, the paclitaxel is administered at an amount of about 150 mg to about 340 mg in combination with Compound A and a checkpoint inhibitor at least 2 times a week.

In some embodiments, the paclitaxel is administered at an amount of about 150 mg to about 330 mg in combination with Compound A and a checkpoint inhibitor at least 2 times a week.

In some embodiments, the paclitaxel is administered at an amount of about 150 mg to about 320 mg in combination with Compound A and a checkpoint inhibitor at least 2 times a week.

In some embodiments, the paclitaxel is administered at an amount of about 150 mg to about 310 mg in combination with Compound A and a checkpoint inhibitor at least 2 times a week.

In some embodiments, the paclitaxel is administered at an amount of about 150 mg to about 300 mg in combination with Compound A and a checkpoint inhibitor at least 2 times a week.

In some embodiments, the paclitaxel is administered at an amount of about 150 mg to about 290 mg in combination with Compound A and a checkpoint inhibitor at least 2 times a week. 8n some embodiments, the paclitaxel is administered at an amount of about 150 mg to about 280 mg in combination with Compound A and a checkpoint inhibitor at least 2 times a week.

In some embodiments, the paclitaxel is administered at an amount of about 150 mg to about 270 mg in combination with Compound A and a checkpoint inhibitor at least 2 times a week.

In some embodiments, the paclitaxel is administered at an amount of about 150 mg to about 260 mg in combination with Compound A and a checkpoint inhibitor at least 2 times a week.

In some embodiments, the paclitaxel is administered at an amount of about 150 mg to about 250 mg in combination with Compound A and a checkpoint inhibitor at least 2 times a week.

In some embodiments, the paclitaxel is administered at an amount of about 200 mg to about 400 mg in combination with Compound A and a checkpoint inhibitor at least 2 times a week.

In some embodiments, the paclitaxel is administered at an amount of about 200 mg to about 350 mg in combination with Compound A and a checkpoint inhibitor at least 2 times a week.

In some embodiments, the paclitaxel is administered at an amount of about 200 mg to about 340 mg in combination with Compound A and a checkpoint inhibitor at least 2 times a week.

In some embodiments, the paclitaxel is administered at an amount of about 200 mg to about 330 mg in combination with Compound A and a checkpoint inhibitor at least 2 times a week.

In some embodiments, the paclitaxel is administered at an amount of about 200 mg to about 320 mg in combination with Compound A and a checkpoint inhibitor at least 2 times a week.

In some embodiments, the paclitaxel is administered at an amount of about 200 mg to about 310 mg in combination with Compound A and a checkpoint inhibitor at least 2 times a week.

In some embodiments, the paclitaxel is administered at an amount of about 200 mg to about 300 mg in combination with Compound A and a checkpoint inhibitor at least 2 times a week.

In some embodiments, the paclitaxel is administered at an amount of about 200 mg to about 295 mg in combination with Compound A and a checkpoint inhibitor at least 2 times a week.

In some embodiments, the paclitaxel is administered at an amount of about 200 mg to about 290 mg in combination with Compound A and a checkpoint inhibitor at least 2 times a week.

In some embodiments, the paclitaxel is administered at an amount of about 200 mg to about 285 mg in combination with Compound A and a checkpoint inhibitor at least 2 times a week.

In some embodiments, the paclitaxel is administered at an amount of about 200 mg to about 280 mg in combination with Compound A and a checkpoint inhibitor at least 2 times a week.

In some embodiments, the paclitaxel is administered at an amount of about 200 mg to about 275 mg in combination with Compound A and a checkpoint inhibitor at least 2 times a week.

In some embodiments, the paclitaxel is administered at an amount of about 200 mg to about 270 mg in combination with Compound A and a checkpoint inhibitor at least 2 times a week.

In some embodiments, the paclitaxel is administered at an amount of about 200 mg to about 265 mg in combination with Compound A and a checkpoint inhibitor at least 2 times a week.

In some embodiments, the paclitaxel is administered at an amount of about 200 mg to about 260 mg in combination with Compound A and a checkpoint inhibitor at least 2 times a week.

In some embodiments, the paclitaxel is administered at an amount of about 200 mg to about 255 mg in combination with Compound A and a checkpoint inhibitor at least 2 times a week.

In some embodiments, the paclitaxel is administered at an amount of about 200 mg to about 250 mg in combination with Compound A and a checkpoint inhibitor at least 2 times a week.

In some embodiments, the paclitaxel is administered at an amount of about 250 mg to about 400 mg in combination with Compound A and a checkpoint inhibitor at least 2 times a week.

In some embodiments, the paclitaxel is administered at an amount of about 250 mg to about 350 mg in combination with Compound A and a checkpoint inhibitor at least 2 times a week.

In some embodiments, the paclitaxel is administered at an amount of about 250 mg to about 340 mg in combination with Compound A and a checkpoint inhibitor at least 2 times a week.

In some embodiments, the paclitaxel is administered at an amount of about 250 mg to about 330 mg in combination with Compound A and a checkpoint inhibitor at least 2 times a week.

In some embodiments, the paclitaxel is administered at an amount of about 250 mg to about 320 mg in combination with Compound A and a checkpoint inhibitor at least 2 times a week.

In some embodiments, the paclitaxel is administered at an amount of about 250 mg to about 310 mg in combination with Compound A and a checkpoint inhibitor at least 2 times a week.

In some embodiments, the paclitaxel is administered at an amount of about 250 mg to about 300 mg in combination with Compound A and a checkpoint inhibitor at least 2 times a week.

In some embodiments, the paclitaxel is administered at an amount of about 250 mg to about 290 mg in combination with Compound A and a checkpoint inhibitor at least 2 times a week.

In some embodiments, the paclitaxel is administered at an amount of about 250 mg to about 280 mg in combination with Compound A and a checkpoint inhibitor at least 2 times a week.

In some embodiments, the paclitaxel is administered at an amount of about 250 mg to about 270 mg in combination with Compound A and a checkpoint inhibitor at least 2 times a week.

In some embodiments, the paclitaxel is administered at an amount of about 250 mg to about 260 mg in combination with Compound A and a checkpoint inhibitor at least 2 times a week.

In some embodiments, the paclitaxel is administered at an amount of about 100 mg in combination with Compound A and a checkpoint inhibitor at least 2 times a week.

In some embodiments, the paclitaxel is administered at an amount of about 105 mg in combination with Compound A and a checkpoint inhibitor at least 2 times a week.

In some embodiments, the paclitaxel is administered at an amount of about 110 mg in combination with Compound A and a checkpoint inhibitor at least 2 times a week.

In some embodiments, the paclitaxel is administered at an amount of about 115 mg in combination with Compound A and a checkpoint inhibitor at least 2 times a week.

In some embodiments, the paclitaxel is administered at an amount of about 120 mg in combination with Compound A and a checkpoint inhibitor at least 2 times a week.

In some embodiments, the paclitaxel is administered at an amount of about 125 mg in combination with Compound A and a checkpoint inhibitor at least 2 times a week.

In some embodiments, the paclitaxel is administered at an amount of about 130 mg in combination with Compound A and a checkpoint inhibitor at least 2 times a week.

In some embodiments, the paclitaxel is administered at an amount of about 135 mg in combination with Compound A and a checkpoint inhibitor at least 2 times a week.

In some embodiments, the paclitaxel is administered at an amount of about 140 mg in combination with Compound A and a checkpoint inhibitor at least 2 times a week.

In some embodiments, the paclitaxel is administered at an amount of about 145 mg in combination with Compound A and a checkpoint inhibitor at least 2 times a week.

In some embodiments, the paclitaxel is administered at an amount of about 150 mg in combination with Compound A and a checkpoint inhibitor at least 2 times a week.

In some embodiments, the paclitaxel is administered at an amount of about 155 mg in combination with Compound A and a checkpoint inhibitor at least 2 times a week.

In some embodiments, the paclitaxel is administered at an amount of about 160 mg in combination with Compound A and a checkpoint inhibitor at least 2 times a week.

In some embodiments, the paclitaxel is administered at an amount of about 165 mg in combination with Compound A and a checkpoint inhibitor at least 2 times a week.

In some embodiments, the paclitaxel is administered at an amount of about 170 mg in combination with Compound A and a checkpoint inhibitor at least 2 times a week.

In some embodiments, the paclitaxel is administered at an amount of about 175 mg in combination with Compound A and a checkpoint inhibitor at least 2 times a week.

In some embodiments, the paclitaxel is administered at an amount of about 180 mg in combination with Compound A and a checkpoint inhibitor at least 2 times a week.

In some embodiments, the paclitaxel is administered at an amount of about 185 mg in combination with Compound A and a checkpoint inhibitor at least 2 times a week.

In some embodiments, the paclitaxel is administered at an amount of about 190 mg in combination with Compound A and a checkpoint inhibitor at least 2 times a week.

In some embodiments, the paclitaxel is administered at an amount of about 195 mg in combination with Compound A and a checkpoint inhibitor at least 2 times a week.

In some embodiments, the paclitaxel is administered at an amount of about 200 mg in combination with Compound A and a checkpoint inhibitor at least 2 times a week.

In some embodiments, the paclitaxel is administered at an amount of about 205 mg in combination with Compound A and a checkpoint inhibitor at least 2 times a week.

In some embodiments, the paclitaxel is administered at an amount of about 210 mg in combination with Compound A and a checkpoint inhibitor at least 2 times a week.

In some embodiments, the paclitaxel is administered at an amount of about 215 mg in combination with Compound A and a checkpoint inhibitor at least 2 times a week.

In some embodiments, the paclitaxel is administered at an amount of about 220 mg in combination with Compound A and a checkpoint inhibitor at least 2 times a week.

In some embodiments, the paclitaxel is administered at an amount of about 225 mg in combination with Compound A and a checkpoint inhibitor at least 2 times a week.

In some embodiments, the paclitaxel is administered at an amount of about 230 mg in combination with Compound A and a checkpoint inhibitor at least 2 times a week.

In some embodiments, the paclitaxel is administered at an amount of about 235 mg in combination with Compound A and a checkpoint inhibitor at least 2 times a week.

In some embodiments, the paclitaxel is administered at an amount of about 240 mg in combination with Compound A and a checkpoint inhibitor at least 2 times a week.

In some embodiments, the paclitaxel is administered at an amount of about 245 mg in combination with Compound A and a checkpoint inhibitor at least 2 times a week.

In some embodiments, the paclitaxel is administered at an amount of about 250 mg in combination with Compound A and a checkpoint inhibitor at least 2 times a week.

In some embodiments, the paclitaxel is administered at an amount of about 255 mg in combination with Compound A and a checkpoint inhibitor at least 2 times a week.

In some embodiments, the paclitaxel is administered at an amount of about 260 mg in combination with Compound A and a checkpoint inhibitor at least 2 times a week.

In some embodiments, the paclitaxel is administered at an amount of about 265 mg in combination with Compound A and a checkpoint inhibitor at least 2 times a week.

In some embodiments, the paclitaxel is administered at an amount of about 270 mg in combination with Compound A and a checkpoint inhibitor at least 2 times a week.

In some embodiments, the paclitaxel is administered at an amount of about 275 mg in combination with Compound A and a checkpoint inhibitor at least 2 times a week.

In some embodiments, the paclitaxel is administered at an amount of about 280 mg in combination with Compound A and a checkpoint inhibitor at least 2 times a week.

In some embodiments, the paclitaxel is administered at an amount of about 285 mg in combination with Compound A and a checkpoint inhibitor at least 2 times a week.

In some embodiments, the paclitaxel is administered at an amount of about 290 mg in combination with Compound A and a checkpoint inhibitor at least 2 times a week.

In some embodiments, the paclitaxel is administered at an amount of about 295 mg in combination with Compound A and a checkpoint inhibitor at least 2 times a week.

In some embodiments, the paclitaxel is administered at an amount of about 300 mg in combination with Compound A and a checkpoint inhibitor at least 2 times a week.

In some embodiments, the paclitaxel is administered at an amount of about 305 mg in combination with Compound A and a checkpoint inhibitor at least 2 times a week.

In some embodiments, the paclitaxel is administered at an amount of about 310 mg in combination with Compound A and a checkpoint inhibitor at least 2 times a week.

In some embodiments, the paclitaxel is administered at an amount of about 315 mg in combination with Compound A and a checkpoint inhibitor at least 2 times a week.

In some embodiments, the paclitaxel is administered at an amount of about 320 mg in combination with Compound A and a checkpoint inhibitor at least 2 times a week.

In some embodiments, the paclitaxel is administered at an amount of about 325 mg in combination with Compound A and a checkpoint inhibitor at least 2 times a week.

In some embodiments, the paclitaxel is administered at an amount of about 330 mg in combination with Compound A and a checkpoint inhibitor at least 2 times a week.

In some embodiments, the paclitaxel is administered at an amount of about 335 mg in combination with Compound A and a checkpoint inhibitor at least 2 times a week.

In some embodiments, the paclitaxel is administered at an amount of about 340 mg in combination with Compound A and a checkpoint inhibitor at least 2 times a week.

In some embodiments, the paclitaxel is administered at an amount of about 345 mg in combination with Compound A and a checkpoint inhibitor at least 2 times a week.

In some embodiments, the paclitaxel is administered at an amount of about 350 mg in combination with Compound A and a checkpoint inhibitor at least 2 times a week.

In some embodiments, the paclitaxel is administered at an amount of about 355 mg in combination with Compound A and a checkpoint inhibitor at least 2 times a week.

In some embodiments, the paclitaxel is administered at an amount of about 360 mg in combination with Compound A and a checkpoint inhibitor at least 2 times a week.

In some embodiments, the paclitaxel is administered at an amount of about 365 mg in combination with Compound A and a checkpoint inhibitor at least 2 times a week.

In some embodiments, the paclitaxel is administered at an amount of about 370 mg in combination with Compound A and a checkpoint inhibitor at least 2 times a week.

In some embodiments, the paclitaxel is administered at an amount of about 375 mg in combination with Compound A and a checkpoint inhibitor at least 2 times a week.

In some embodiments, the paclitaxel is administered at an amount of about 380 mg in combination with Compound A and a checkpoint inhibitor at least 2 times a week.

In some embodiments, the paclitaxel is administered at an amount of about 385 mg in combination with Compound A and a checkpoint inhibitor at least 2 times a week.

In some embodiments, the paclitaxel is administered at an amount of about 390 mg in combination with Compound A and a checkpoint inhibitor at least 2 times a week.

In some embodiments, the paclitaxel is administered at an amount of about 395 mg in combination with Compound A and a checkpoint inhibitor at least 2 times a week.

In some embodiments, the paclitaxel is administered at an amount of about 400 mg in combination with Compound A and a checkpoint inhibitor at least 2 times a week.

In some embodiments, the paclitaxel is administered in combination with Compound A and a checkpoint inhibitor at least 2 times a week, wherein the at least 2 days are consecutive.

In some embodiments, the paclitaxel is administered in combination with Compound A and a checkpoint inhibitor at least 3 times a week.

In some embodiments, the paclitaxel is administered at an amount of about 100 mg to about 400 mg in combination with Compound A and a checkpoint inhibitor at least 3 times a week.

In some embodiments, the paclitaxel is administered at an amount of about 100 mg to about 350 mg in combination with Compound A and a checkpoint inhibitor at least 3 times a week.

In some embodiments, the paclitaxel is administered at an amount of about 100 mg to about 300 mg in combination with Compound A and a checkpoint inhibitor at least 3 times a week.

In some embodiments, the paclitaxel is administered at an amount of about 150 mg to about 400 mg in combination with Compound A and a checkpoint inhibitor at least 3 times a week.

In some embodiments, the paclitaxel is administered at an amount of about 150 mg to about 350 mg in combination with Compound A and a checkpoint inhibitor at least 3 times a week.

In some embodiments, the paclitaxel is administered at an amount of about 150 mg to about 340 mg in combination with Compound A and a checkpoint inhibitor at least 3 times a week.

In some embodiments, the paclitaxel is administered at an amount of about 150 mg to about 330 mg in combination with Compound A and a checkpoint inhibitor at least 3 times a week.

In some embodiments, the paclitaxel is administered at an amount of about 150 mg to about 320 mg in combination with Compound A and a checkpoint inhibitor at least 3 times a week.

In some embodiments, the paclitaxel is administered at an amount of about 150 mg to about 310 mg in combination with Compound A and a checkpoint inhibitor at least 3 times a week.

In some embodiments, the paclitaxel is administered at an amount of about 150 mg to about 300 mg in combination with Compound A and a checkpoint inhibitor at least 3 times a week.

In some embodiments, the paclitaxel is administered at an amount of about 150 mg to about 290 mg in combination with Compound A and a checkpoint inhibitor at least 3 times a week. 8n some embodiments, the paclitaxel is administered at an amount of about 150 mg to about 280 mg in combination with Compound A and a checkpoint inhibitor at least 3 times a week.

In some embodiments, the paclitaxel is administered at an amount of about 150 mg to about 270 mg in combination with Compound A and a checkpoint inhibitor at least 3 times a week.

In some embodiments, the paclitaxel is administered at an amount of about 150 mg to about 260 mg in combination with Compound A and a checkpoint inhibitor at least 3 times a week.

In some embodiments, the paclitaxel is administered at an amount of about 150 mg to about 250 mg in combination with Compound A and a checkpoint inhibitor at least 3 times a week.

In some embodiments, the paclitaxel is administered at an amount of about 200 mg to about 400 mg in combination with Compound A and a checkpoint inhibitor at least 3 times a week.

In some embodiments, the paclitaxel is administered at an amount of about 200 mg to about 350 mg in combination with Compound A and a checkpoint inhibitor at least 3 times a week.

In some embodiments, the paclitaxel is administered at an amount of about 200 mg to about 340 mg in combination with Compound A and a checkpoint inhibitor at least 3 times a week.

In some embodiments, the paclitaxel is administered at an amount of about 200 mg to about 330 mg in combination with Compound A and a checkpoint inhibitor at least 3 times a week.

In some embodiments, the paclitaxel is administered at an amount of about 200 mg to about 320 mg in combination with Compound A and a checkpoint inhibitor at least 3 times a week.

In some embodiments, the paclitaxel is administered at an amount of about 200 mg to about 310 mg in combination with Compound A and a checkpoint inhibitor at least 3 times a week.

In some embodiments, the paclitaxel is administered at an amount of about 200 mg to about 300 mg in combination with Compound A and a checkpoint inhibitor at least 3 times a week.

In some embodiments, the paclitaxel is administered at an amount of about 200 mg to about 295 mg in combination with Compound A and a checkpoint inhibitor at least 3 times a week.

In some embodiments, the paclitaxel is administered at an amount of about 200 mg to about 290 mg in combination with Compound A and a checkpoint inhibitor at least 3 times a week.

In some embodiments, the paclitaxel is administered at an amount of about 200 mg to about 285 mg in combination with Compound A and a checkpoint inhibitor at least 3 times a week.

In some embodiments, the paclitaxel is administered at an amount of about 200 mg to about 280 mg in combination with Compound A and a checkpoint inhibitor at least 3 times a week.

In some embodiments, the paclitaxel is administered at an amount of about 200 mg to about 275 mg in combination with Compound A and a checkpoint inhibitor at least 3 times a week.

In some embodiments, the paclitaxel is administered at an amount of about 200 mg to about 270 mg in combination with Compound A and a checkpoint inhibitor at least 3 times a week.

In some embodiments, the paclitaxel is administered at an amount of about 200 mg to about 265 mg in combination with Compound A and a checkpoint inhibitor at least 3 times a week.

In some embodiments, the paclitaxel is administered at an amount of about 200 mg to about 260 mg in combination with Compound A and a checkpoint inhibitor at least 3 times a week.

In some embodiments, the paclitaxel is administered at an amount of about 200 mg to about 255 mg in combination with Compound A and a checkpoint inhibitor at least 3 times a week.

In some embodiments, the paclitaxel is administered at an amount of about 200 mg to about 250 mg in combination with Compound A and a checkpoint inhibitor at least 3 times a week.

In some embodiments, the paclitaxel is administered at an amount of about 250 mg to about 400 mg in combination with Compound A and a checkpoint inhibitor at least 3 times a week.

In some embodiments, the paclitaxel is administered at an amount of about 250 mg to about 350 mg in combination with Compound A and a checkpoint inhibitor at least 3 times a week.

In some embodiments, the paclitaxel is administered at an amount of about 250 mg to about 340 mg in combination with Compound A and a checkpoint inhibitor at least 3 times a week.

In some embodiments, the paclitaxel is administered at an amount of about 250 mg to about 330 mg in combination with Compound A and a checkpoint inhibitor at least 3 times a week.

In some embodiments, the paclitaxel is administered at an amount of about 250 mg to about 320 mg in combination with Compound A and a checkpoint inhibitor at least 3 times a week.

In some embodiments, the paclitaxel is administered at an amount of about 250 mg to about 310 mg in combination with Compound A and a checkpoint inhibitor at least 3 times a week.

In some embodiments, the paclitaxel is administered at an amount of about 250 mg to about 300 mg in combination with Compound A and a checkpoint inhibitor at least 3 times a week.

In some embodiments, the paclitaxel is administered at an amount of about 250 mg to about 290 mg in combination with Compound A and a checkpoint inhibitor at least 3 times a week.

In some embodiments, the paclitaxel is administered at an amount of about 250 mg to about 280 mg in combination with Compound A and a checkpoint inhibitor at least 3 times a week.

In some embodiments, the paclitaxel is administered at an amount of about 250 mg to about 270 mg in combination with Compound A and a checkpoint inhibitor at least 3 times a week.

In some embodiments, the paclitaxel is administered at an amount of about 250 mg to about 260 mg in combination with Compound A and a checkpoint inhibitor at least 3 times a week.

In some embodiments, the paclitaxel is administered at an amount of about 100 mg in combination with Compound A and a checkpoint inhibitor at least 3 times a week.

In some embodiments, the paclitaxel is administered at an amount of about 105 mg in combination with Compound A and a checkpoint inhibitor at least 3 times a week.

In some embodiments, the paclitaxel is administered at an amount of about 110 mg in combination with Compound A and a checkpoint inhibitor at least 3 times a week.

In some embodiments, the paclitaxel is administered at an amount of about 115 mg in combination with Compound A and a checkpoint inhibitor at least 3 times a week.

In some embodiments, the paclitaxel is administered at an amount of about 120 mg in combination with Compound A and a checkpoint inhibitor at least 3 times a week.

In some embodiments, the paclitaxel is administered at an amount of about 125 mg in combination with Compound A and a checkpoint inhibitor at least 3 times a week.

In some embodiments, the paclitaxel is administered at an amount of about 130 mg in combination with Compound A and a checkpoint inhibitor at least 3 times a week.

In some embodiments, the paclitaxel is administered at an amount of about 135 mg in combination with Compound A and a checkpoint inhibitor at least 3 times a week.

In some embodiments, the paclitaxel is administered at an amount of about 140 mg in combination with Compound A and a checkpoint inhibitor at least 3 times a week.

In some embodiments, the paclitaxel is administered at an amount of about 145 mg in combination with Compound A and a checkpoint inhibitor at least 3 times a week.

In some embodiments, the paclitaxel is administered at an amount of about 150 mg in combination with Compound A and a checkpoint inhibitor at least 3 times a week.

In some embodiments, the paclitaxel is administered at an amount of about 155 mg in combination with Compound A and a checkpoint inhibitor at least 3 times a week.

In some embodiments, the paclitaxel is administered at an amount of about 160 mg in combination with Compound A and a checkpoint inhibitor at least 3 times a week.

In some embodiments, the paclitaxel is administered at an amount of about 165 mg in combination with Compound A and a checkpoint inhibitor at least 3 times a week.

In some embodiments, the paclitaxel is administered at an amount of about 170 mg in combination with Compound A and a checkpoint inhibitor at least 3 times a week.

In some embodiments, the paclitaxel is administered at an amount of about 175 mg in combination with Compound A and a checkpoint inhibitor at least 3 times a week.

In some embodiments, the paclitaxel is administered at an amount of about 180 mg in combination with Compound A and a checkpoint inhibitor at least 3 times a week.

In some embodiments, the paclitaxel is administered at an amount of about 185 mg in combination with Compound A and a checkpoint inhibitor at least 3 times a week.

In some embodiments, the paclitaxel is administered at an amount of about 190 mg in combination with Compound A and a checkpoint inhibitor at least 3 times a week.

In some embodiments, the paclitaxel is administered at an amount of about 195 mg in combination with Compound A and a checkpoint inhibitor at least 3 times a week.

In some embodiments, the paclitaxel is administered at an amount of about 200 mg in combination with Compound A and a checkpoint inhibitor at least 3 times a week.

In some embodiments, the paclitaxel is administered at an amount of about 205 mg in combination with Compound A and a checkpoint inhibitor at least 3 times a week.

In some embodiments, the paclitaxel is administered at an amount of about 210 mg in combination with Compound A and a checkpoint inhibitor at least 3 times a week.

In some embodiments, the paclitaxel is administered at an amount of about 215 mg in combination with Compound A and a checkpoint inhibitor at least 3 times a week.

In some embodiments, the paclitaxel is administered at an amount of about 220 mg in combination with Compound A and a checkpoint inhibitor at least 3 times a week.

In some embodiments, the paclitaxel is administered at an amount of about 225 mg in combination with Compound A and a checkpoint inhibitor at least 3 times a week.

In some embodiments, the paclitaxel is administered at an amount of about 230 mg in combination with Compound A and a checkpoint inhibitor at least 3 times a week.

In some embodiments, the paclitaxel is administered at an amount of about 235 mg in combination with Compound A and a checkpoint inhibitor at least 3 times a week.

In some embodiments, the paclitaxel is administered at an amount of about 240 mg in combination with Compound A and a checkpoint inhibitor at least 3 times a week.

In some embodiments, the paclitaxel is administered at an amount of about 245 mg in combination with Compound A and a checkpoint inhibitor at least 3 times a week.

In some embodiments, the paclitaxel is administered at an amount of about 250 mg in combination with Compound A and a checkpoint inhibitor at least 3 times a week.

In some embodiments, the paclitaxel is administered at an amount of about 255 mg in combination with Compound A and a checkpoint inhibitor at least 3 times a week.

In some embodiments, the paclitaxel is administered at an amount of about 260 mg in combination with Compound A and a checkpoint inhibitor at least 3 times a week.

In some embodiments, the paclitaxel is administered at an amount of about 265 mg in combination with Compound A and a checkpoint inhibitor at least 3 times a week.

In some embodiments, the paclitaxel is administered at an amount of about 270 mg in combination with Compound A and a checkpoint inhibitor at least 3 times a week.

In some embodiments, the paclitaxel is administered at an amount of about 275 mg in combination with Compound A and a checkpoint inhibitor at least 3 times a week.

In some embodiments, the paclitaxel is administered at an amount of about 280 mg in combination with Compound A and a checkpoint inhibitor at least 3 times a week.

In some embodiments, the paclitaxel is administered at an amount of about 285 mg in combination with Compound A and a checkpoint inhibitor at least 3 times a week.

In some embodiments, the paclitaxel is administered at an amount of about 290 mg in combination with Compound A and a checkpoint inhibitor at least 3 times a week.

In some embodiments, the paclitaxel is administered at an amount of about 295 mg in combination with Compound A and a checkpoint inhibitor at least 3 times a week.

In some embodiments, the paclitaxel is administered at an amount of about 300 mg in combination with Compound A and a checkpoint inhibitor at least 3 times a week.

In some embodiments, the paclitaxel is administered at an amount of about 305 mg in combination with Compound A and a checkpoint inhibitor at least 3 times a week.

In some embodiments, the paclitaxel is administered at an amount of about 310 mg in combination with Compound A and a checkpoint inhibitor at least 3 times a week.

In some embodiments, the paclitaxel is administered at an amount of about 315 mg in combination with Compound A and a checkpoint inhibitor at least 3 times a week.

In some embodiments, the paclitaxel is administered at an amount of about 320 mg in combination with Compound A and a checkpoint inhibitor at least 3 times a week.

In some embodiments, the paclitaxel is administered at an amount of about 325 mg in combination with Compound A and a checkpoint inhibitor at least 3 times a week.

In some embodiments, the paclitaxel is administered at an amount of about 330 mg in combination with Compound A and a checkpoint inhibitor at least 3 times a week.

In some embodiments, the paclitaxel is administered at an amount of about 335 mg in combination with Compound A and a checkpoint inhibitor at least 3 times a week.

In some embodiments, the paclitaxel is administered at an amount of about 340 mg in combination with Compound A and a checkpoint inhibitor at least 3 times a week.

In some embodiments, the paclitaxel is administered at an amount of about 345 mg in combination with Compound A and a checkpoint inhibitor at least 3 times a week.

In some embodiments, the paclitaxel is administered at an amount of about 350 mg in combination with Compound A and a checkpoint inhibitor at least 3 times a week.

In some embodiments, the paclitaxel is administered at an amount of about 355 mg in combination with Compound A and a checkpoint inhibitor at least 3 times a week.

In some embodiments, the paclitaxel is administered at an amount of about 360 mg in combination with Compound A and a checkpoint inhibitor at least 3 times a week.

In some embodiments, the paclitaxel is administered at an amount of about 365 mg in combination with Compound A and a checkpoint inhibitor at least 3 times a week.

In some embodiments, the paclitaxel is administered at an amount of about 370 mg in combination with Compound A and a checkpoint inhibitor at least 3 times a week.

In some embodiments, the paclitaxel is administered at an amount of about 375 mg in combination with Compound A and a checkpoint inhibitor at least 3 times a week.

In some embodiments, the paclitaxel is administered at an amount of about 380 mg in combination with Compound A and a checkpoint inhibitor at least 3 times a week.

In some embodiments, the paclitaxel is administered at an amount of about 385 mg in combination with Compound A and a checkpoint inhibitor at least 3 times a week.

In some embodiments, the paclitaxel is administered at an amount of about 390 mg in combination with Compound A and a checkpoint inhibitor at least 3 times a week.

In some embodiments, the paclitaxel is administered at an amount of about 395 mg in combination with Compound A and a checkpoint inhibitor at least 3 times a week.

In some embodiments, the paclitaxel is administered at an amount of about 400 mg in combination with Compound A and a checkpoint inhibitor at least 3 times a week.

In some embodiments, the paclitaxel is administered in combination with Compound A and a checkpoint inhibitor at least 3 times a week, wherein the at least 3 days are consecutive.

In some embodiments, the paclitaxel is administered in combination with Compound A and a checkpoint inhibitor at least 4 times a week.

In some embodiments, the paclitaxel is administered at an amount of about 100 mg to about 400 mg in combination with Compound A and a checkpoint inhibitor at least 4 times a week.

In some embodiments, the paclitaxel is administered at an amount of about 100 mg to about 350 mg in combination with Compound A and a checkpoint inhibitor at least 4 times a week.

In some embodiments, the paclitaxel is administered at an amount of about 100 mg to about 300 mg in combination with Compound A and a checkpoint inhibitor at least 4 times a week.

In some embodiments, the paclitaxel is administered at an amount of about 150 mg to about 400 mg in combination with Compound A and a checkpoint inhibitor at least 4 times a week.

In some embodiments, the paclitaxel is administered at an amount of about 150 mg to about 350 mg in combination with Compound A and a checkpoint inhibitor at least 4 times a week.

In some embodiments, the paclitaxel is administered at an amount of about 150 mg to about 340 mg in combination with Compound A and a checkpoint inhibitor at least 4 times a week.

In some embodiments, the paclitaxel is administered at an amount of about 150 mg to about 330 mg in combination with Compound A and a checkpoint inhibitor at least 4 times a week.

In some embodiments, the paclitaxel is administered at an amount of about 150 mg to about 320 mg in combination with Compound A and a checkpoint inhibitor at least 4 times a week.

In some embodiments, the paclitaxel is administered at an amount of about 150 mg to about 310 mg in combination with Compound A and a checkpoint inhibitor at least 4 times a week.

In some embodiments, the paclitaxel is administered at an amount of about 150 mg to about 300 mg in combination with Compound A and a checkpoint inhibitor at least 4 times a week.

In some embodiments, the paclitaxel is administered at an amount of about 150 mg to about 290 mg in combination with Compound A and a checkpoint inhibitor at least 4 times a week. 8n some embodiments, the paclitaxel is administered at an amount of about 150 mg to about 280 mg in combination with Compound A and a checkpoint inhibitor at least 4 times a week.

In some embodiments, the paclitaxel is administered at an amount of about 150 mg to about 270 mg in combination with Compound A and a checkpoint inhibitor at least 4 times a week.

In some embodiments, the paclitaxel is administered at an amount of about 150 mg to about 260 mg in combination with Compound A and a checkpoint inhibitor at least 4 times a week.

In some embodiments, the paclitaxel is administered at an amount of about 150 mg to about 250 mg in combination with Compound A and a checkpoint inhibitor at least 4 times a week.

In some embodiments, the paclitaxel is administered at an amount of about 200 mg to about 400 mg in combination with Compound A and a checkpoint inhibitor at least 4 times a week.

In some embodiments, the paclitaxel is administered at an amount of about 200 mg to about 350 mg in combination with Compound A and a checkpoint inhibitor at least 4 times a week.

In some embodiments, the paclitaxel is administered at an amount of about 200 mg to about 340 mg in combination with Compound A and a checkpoint inhibitor at least 4 times a week.

In some embodiments, the paclitaxel is administered at an amount of about 200 mg to about 330 mg in combination with Compound A and a checkpoint inhibitor at least 4 times a week.

In some embodiments, the paclitaxel is administered at an amount of about 200 mg to about 320 mg in combination with Compound A and a checkpoint inhibitor at least 4 times a week.

In some embodiments, the paclitaxel is administered at an amount of about 200 mg to about 310 mg in combination with Compound A and a checkpoint inhibitor at least 4 times a week.

In some embodiments, the paclitaxel is administered at an amount of about 200 mg to about 300 mg in combination with Compound A and a checkpoint inhibitor at least 4 times a week.

In some embodiments, the paclitaxel is administered at an amount of about 200 mg to about 295 mg in combination with Compound A and a checkpoint inhibitor at least 4 times a week.

In some embodiments, the paclitaxel is administered at an amount of about 200 mg to about 290 mg in combination with Compound A and a checkpoint inhibitor at least 4 times a week.

In some embodiments, the paclitaxel is administered at an amount of about 200 mg to about 285 mg in combination with Compound A and a checkpoint inhibitor at least 4 times a week.

In some embodiments, the paclitaxel is administered at an amount of about 200 mg to about 280 mg in combination with Compound A and a checkpoint inhibitor at least 4 times a week.

In some embodiments, the paclitaxel is administered at an amount of about 200 mg to about 275 mg in combination with Compound A and a checkpoint inhibitor at least 4 times a week.

In some embodiments, the paclitaxel is administered at an amount of about 200 mg to about 270 mg in combination with Compound A and a checkpoint inhibitor at least 4 times a week.

In some embodiments, the paclitaxel is administered at an amount of about 200 mg to about 265 mg in combination with Compound A and a checkpoint inhibitor at least 4 times a week.

In some embodiments, the paclitaxel is administered at an amount of about 200 mg to about 260 mg in combination with Compound A and a checkpoint inhibitor at least 4 times a week.

In some embodiments, the paclitaxel is administered at an amount of about 200 mg to about 255 mg in combination with Compound A and a checkpoint inhibitor at least 4 times a week.

In some embodiments, the paclitaxel is administered at an amount of about 200 mg to about 250 mg in combination with Compound A and a checkpoint inhibitor at least 4 times a week.

In some embodiments, the paclitaxel is administered at an amount of about 250 mg to about 400 mg in combination with Compound A and a checkpoint inhibitor at least 4 times a week.

In some embodiments, the paclitaxel is administered at an amount of about 250 mg to about 350 mg in combination with Compound A and a checkpoint inhibitor at least 4 times a week.

In some embodiments, the paclitaxel is administered at an amount of about 250 mg to about 340 mg in combination with Compound A and a checkpoint inhibitor at least 4 times a week.

In some embodiments, the paclitaxel is administered at an amount of about 250 mg to about 330 mg in combination with Compound A and a checkpoint inhibitor at least 4 times a week.

In some embodiments, the paclitaxel is administered at an amount of about 250 mg to about 320 mg in combination with Compound A and a checkpoint inhibitor at least 4 times a week.

In some embodiments, the paclitaxel is administered at an amount of about 250 mg to about 310 mg in combination with Compound A and a checkpoint inhibitor at least 4 times a week.

In some embodiments, the paclitaxel is administered at an amount of about 250 mg to about 300 mg in combination with Compound A and a checkpoint inhibitor at least 4 times a week.

In some embodiments, the paclitaxel is administered at an amount of about 250 mg to about 290 mg in combination with Compound A and a checkpoint inhibitor at least 4 times a week.

In some embodiments, the paclitaxel is administered at an amount of about 250 mg to about 280 mg in combination with Compound A and a checkpoint inhibitor at least 4 times a week.

In some embodiments, the paclitaxel is administered at an amount of about 250 mg to about 270 mg in combination with Compound A and a checkpoint inhibitor at least 4 times a week.

In some embodiments, the paclitaxel is administered at an amount of about 250 mg to about 260 mg in combination with Compound A and a checkpoint inhibitor at least 4 times a week.

In some embodiments, the paclitaxel is administered at an amount of about 100 mg in combination with Compound A and a checkpoint inhibitor at least 4 times a week.

In some embodiments, the paclitaxel is administered at an amount of about 105 mg in combination with Compound A and a checkpoint inhibitor at least 4 times a week.

In some embodiments, the paclitaxel is administered at an amount of about 110 mg in combination with Compound A and a checkpoint inhibitor at least 4 times a week.

In some embodiments, the paclitaxel is administered at an amount of about 115 mg in combination with Compound A and a checkpoint inhibitor at least 4 times a week.

In some embodiments, the paclitaxel is administered at an amount of about 120 mg in combination with Compound A and a checkpoint inhibitor at least 4 times a week.

In some embodiments, the paclitaxel is administered at an amount of about 125 mg in combination with Compound A and a checkpoint inhibitor at least 4 times a week.

In some embodiments, the paclitaxel is administered at an amount of about 130 mg in combination with Compound A and a checkpoint inhibitor at least 4 times a week.

In some embodiments, the paclitaxel is administered at an amount of about 135 mg in combination with Compound A and a checkpoint inhibitor at least 4 times a week.

In some embodiments, the paclitaxel is administered at an amount of about 140 mg in combination with Compound A and a checkpoint inhibitor at least 4 times a week.

In some embodiments, the paclitaxel is administered at an amount of about 145 mg in combination with Compound A and a checkpoint inhibitor at least 4 times a week.

In some embodiments, the paclitaxel is administered at an amount of about 150 mg in combination with Compound A and a checkpoint inhibitor at least 4 times a week.

In some embodiments, the paclitaxel is administered at an amount of about 155 mg in combination with Compound A and a checkpoint inhibitor at least 4 times a week.

In some embodiments, the paclitaxel is administered at an amount of about 160 mg in combination with Compound A and a checkpoint inhibitor at least 4 times a week.

In some embodiments, the paclitaxel is administered at an amount of about 165 mg in combination with Compound A and a checkpoint inhibitor at least 4 times a week.

In some embodiments, the paclitaxel is administered at an amount of about 170 mg in combination with Compound A and a checkpoint inhibitor at least 4 times a week.

In some embodiments, the paclitaxel is administered at an amount of about 175 mg in combination with Compound A and a checkpoint inhibitor at least 4 times a week.

In some embodiments, the paclitaxel is administered at an amount of about 180 mg in combination with Compound A and a checkpoint inhibitor at least 4 times a week.

In some embodiments, the paclitaxel is administered at an amount of about 185 mg in combination with Compound A and a checkpoint inhibitor at least 4 times a week.

In some embodiments, the paclitaxel is administered at an amount of about 190 mg in combination with Compound A and a checkpoint inhibitor at least 4 times a week.

In some embodiments, the paclitaxel is administered at an amount of about 195 mg in combination with Compound A and a checkpoint inhibitor at least 4 times a week.

In some embodiments, the paclitaxel is administered at an amount of about 200 mg in combination with Compound A and a checkpoint inhibitor at least 4 times a week.

In some embodiments, the paclitaxel is administered at an amount of about 205 mg in combination with Compound A and a checkpoint inhibitor at least 4 times a week.

In some embodiments, the paclitaxel is administered at an amount of about 210 mg in combination with Compound A and a checkpoint inhibitor at least 4 times a week.

In some embodiments, the paclitaxel is administered at an amount of about 215 mg in combination with Compound A and a checkpoint inhibitor at least 4 times a week.

In some embodiments, the paclitaxel is administered at an amount of about 220 mg in combination with Compound A and a checkpoint inhibitor at least 4 times a week.

In some embodiments, the paclitaxel is administered at an amount of about 225 mg in combination with Compound A and a checkpoint inhibitor at least 4 times a week.

In some embodiments, the paclitaxel is administered at an amount of about 230 mg in combination with Compound A and a checkpoint inhibitor at least 4 times a week.

In some embodiments, the paclitaxel is administered at an amount of about 235 mg in combination with Compound A and a checkpoint inhibitor at least 4 times a week.

In some embodiments, the paclitaxel is administered at an amount of about 240 mg in combination with Compound A and a checkpoint inhibitor at least 4 times a week.

In some embodiments, the paclitaxel is administered at an amount of about 245 mg in combination with Compound A and a checkpoint inhibitor at least 4 times a week.

In some embodiments, the paclitaxel is administered at an amount of about 250 mg in combination with Compound A and a checkpoint inhibitor at least 4 times a week.

In some embodiments, the paclitaxel is administered at an amount of about 255 mg in combination with Compound A and a checkpoint inhibitor at least 4 times a week.

In some embodiments, the paclitaxel is administered at an amount of about 260 mg in combination with Compound A and a checkpoint inhibitor at least 4 times a week.

In some embodiments, the paclitaxel is administered at an amount of about 265 mg in combination with Compound A and a checkpoint inhibitor at least 4 times a week.

In some embodiments, the paclitaxel is administered at an amount of about 270 mg in combination with Compound A and a checkpoint inhibitor at least 4 times a week.

In some embodiments, the paclitaxel is administered at an amount of about 275 mg in combination with Compound A and a checkpoint inhibitor at least 4 times a week.

In some embodiments, the paclitaxel is administered at an amount of about 280 mg in combination with Compound A and a checkpoint inhibitor at least 4 times a week.

In some embodiments, the paclitaxel is administered at an amount of about 285 mg in combination with Compound A and a checkpoint inhibitor at least 4 times a week.

In some embodiments, the paclitaxel is administered at an amount of about 290 mg in combination with Compound A and a checkpoint inhibitor at least 4 times a week.

In some embodiments, the paclitaxel is administered at an amount of about 295 mg in combination with Compound A and a checkpoint inhibitor at least 4 times a week.

In some embodiments, the paclitaxel is administered at an amount of about 300 mg in combination with Compound A and a checkpoint inhibitor at least 4 times a week.

In some embodiments, the paclitaxel is administered at an amount of about 305 mg in combination with Compound A and a checkpoint inhibitor at least 4 times a week.

In some embodiments, the paclitaxel is administered at an amount of about 310 mg in combination with Compound A and a checkpoint inhibitor at least 4 times a week.

In some embodiments, the paclitaxel is administered at an amount of about 315 mg in combination with Compound A and a checkpoint inhibitor at least 4 times a week.

In some embodiments, the paclitaxel is administered at an amount of about 320 mg in combination with Compound A and a checkpoint inhibitor at least 4 times a week.

In some embodiments, the paclitaxel is administered at an amount of about 325 mg in combination with Compound A and a checkpoint inhibitor at least 4 times a week.

In some embodiments, the paclitaxel is administered at an amount of about 330 mg in combination with Compound A and a checkpoint inhibitor at least 4 times a week.

In some embodiments, the paclitaxel is administered at an amount of about 335 mg in combination with Compound A and a checkpoint inhibitor at least 4 times a week.

In some embodiments, the paclitaxel is administered at an amount of about 340 mg in combination with Compound A and a checkpoint inhibitor at least 4 times a week.

In some embodiments, the paclitaxel is administered at an amount of about 345 mg in combination with Compound A and a checkpoint inhibitor at least 4 times a week.

In some embodiments, the paclitaxel is administered at an amount of about 350 mg in combination with Compound A and a checkpoint inhibitor at least 4 times a week.

In some embodiments, the paclitaxel is administered at an amount of about 355 mg in combination with Compound A and a checkpoint inhibitor at least 4 times a week.

In some embodiments, the paclitaxel is administered at an amount of about 360 mg in combination with Compound A and a checkpoint inhibitor at least 4 times a week.

In some embodiments, the paclitaxel is administered at an amount of about 365 mg in combination with Compound A and a checkpoint inhibitor at least 4 times a week.

In some embodiments, the paclitaxel is administered at an amount of about 370 mg in combination with Compound A and a checkpoint inhibitor at least 4 times a week.

In some embodiments, the paclitaxel is administered at an amount of about 375 mg in combination with Compound A and a checkpoint inhibitor at least 4 times a week.

In some embodiments, the paclitaxel is administered at an amount of about 380 mg in combination with Compound A and a checkpoint inhibitor at least 4 times a week.

In some embodiments, the paclitaxel is administered at an amount of about 385 mg in combination with Compound A and a checkpoint inhibitor at least 4 times a week.

In some embodiments, the paclitaxel is administered at an amount of about 390 mg in combination with Compound A and a checkpoint inhibitor at least 4 times a week.

In some embodiments, the paclitaxel is administered at an amount of about 395 mg in combination with Compound A and a checkpoint inhibitor at least 4 times a week.

In some embodiments, the paclitaxel is administered at an amount of about 400 mg in combination with Compound A and a checkpoint inhibitor at least 4 times a week.

In some embodiments, the paclitaxel is administered in combination with Compound A and a checkpoint inhibitor at least 4 times a week, wherein the at least 4 days are consecutive.

In some embodiments, the paclitaxel is administered in combination with Compound A and a checkpoint inhibitor at least 5 times a week.

In some embodiments, the paclitaxel is administered at an amount of about 100 mg to about 400 mg in combination with Compound A and a checkpoint inhibitor at least 5 times a week.

In some embodiments, the paclitaxel is administered at an amount of about 100 mg to about 350 mg in combination with Compound A and a checkpoint inhibitor at least 5 times a week.

In some embodiments, the paclitaxel is administered at an amount of about 100 mg to about 300 mg in combination with Compound A and a checkpoint inhibitor at least 5 times a week.

In some embodiments, the paclitaxel is administered at an amount of about 150 mg to about 400 mg in combination with Compound A and a checkpoint inhibitor at least 5 times a week.

In some embodiments, the paclitaxel is administered at an amount of about 150 mg to about 350 mg in combination with Compound A and a checkpoint inhibitor at least 5 times a week.

In some embodiments, the paclitaxel is administered at an amount of about 150 mg to about 340 mg in combination with Compound A and a checkpoint inhibitor at least 5 times a week.

In some embodiments, the paclitaxel is administered at an amount of about 150 mg to about 330 mg in combination with Compound A and a checkpoint inhibitor at least 5 times a week.

In some embodiments, the paclitaxel is administered at an amount of about 150 mg to about 320 mg in combination with Compound A and a checkpoint inhibitor at least 5 times a week.

In some embodiments, the paclitaxel is administered at an amount of about 150 mg to about 310 mg in combination with Compound A and a checkpoint inhibitor at least 5 times a week.

In some embodiments, the paclitaxel is administered at an amount of about 150 mg to about 300 mg in combination with Compound A and a checkpoint inhibitor at least 5 times a week.

In some embodiments, the paclitaxel is administered at an amount of about 150 mg to about 290 mg in combination with Compound A and a checkpoint inhibitor at least 5 times a week.

8n some embodiments, the paclitaxel is administered at an amount of about 150 mg to about 280 mg in combination with Compound A and a checkpoint inhibitor at least 5 times a week.

In some embodiments, the paclitaxel is administered at an amount of about 150 mg to about 270 mg in combination with Compound A and a checkpoint inhibitor at least 5 times a week.

In some embodiments, the paclitaxel is administered at an amount of about 150 mg to about 260 mg in combination with Compound A and a checkpoint inhibitor at least 5 times a week.

In some embodiments, the paclitaxel is administered at an amount of about 150 mg to about 250 mg in combination with Compound A and a checkpoint inhibitor at least 5 times a week.

In some embodiments, the paclitaxel is administered at an amount of about 200 mg to about 400 mg in combination with Compound A and a checkpoint inhibitor at least 5 times a week.

In some embodiments, the paclitaxel is administered at an amount of about 200 mg to about 350 mg in combination with Compound A and a checkpoint inhibitor at least 5 times a week.

In some embodiments, the paclitaxel is administered at an amount of about 200 mg to about 340 mg in combination with Compound A and a checkpoint inhibitor at least 5 times a week.

In some embodiments, the paclitaxel is administered at an amount of about 200 mg to about 330 mg in combination with Compound A and a checkpoint inhibitor at least 5 times a week.

In some embodiments, the paclitaxel is administered at an amount of about 200 mg to about 320 mg in combination with Compound A and a checkpoint inhibitor at least 5 times a week.

In some embodiments, the paclitaxel is administered at an amount of about 200 mg to about 310 mg in combination with Compound A and a checkpoint inhibitor at least 5 times a week.

In some embodiments, the paclitaxel is administered at an amount of about 200 mg to about 300 mg in combination with Compound A and a checkpoint inhibitor at least 5 times a week.

In some embodiments, the paclitaxel is administered at an amount of about 200 mg to about 295 mg in combination with Compound A and a checkpoint inhibitor at least 5 times a week.

In some embodiments, the paclitaxel is administered at an amount of about 200 mg to about 290 mg in combination with Compound A and a checkpoint inhibitor at least 5 times a week.

In some embodiments, the paclitaxel is administered at an amount of about 200 mg to about 285 mg in combination with Compound A and a checkpoint inhibitor at least 5 times a week.

In some embodiments, the paclitaxel is administered at an amount of about 200 mg to about 280 mg in combination with Compound A and a checkpoint inhibitor at least 5 times a week.

In some embodiments, the paclitaxel is administered at an amount of about 200 mg to about 275 mg in combination with Compound A and a checkpoint inhibitor at least 5 times a week.

In some embodiments, the paclitaxel is administered at an amount of about 200 mg to about 270 mg in combination with Compound A and a checkpoint inhibitor at least 5 times a week.

In some embodiments, the paclitaxel is administered at an amount of about 200 mg to about 265 mg in combination with Compound A and a checkpoint inhibitor at least 5 times a week.

In some embodiments, the paclitaxel is administered at an amount of about 200 mg to about 260 mg in combination with Compound A and a checkpoint inhibitor at least 5 times a week.

In some embodiments, the paclitaxel is administered at an amount of about 200 mg to about 255 mg in combination with Compound A and a checkpoint inhibitor at least 5 times a week.

In some embodiments, the paclitaxel is administered at an amount of about 200 mg to about 250 mg in combination with Compound A and a checkpoint inhibitor at least 5 times a week.

In some embodiments, the paclitaxel is administered at an amount of about 250 mg to about 400 mg in combination with Compound A and a checkpoint inhibitor at least 5 times a week.

In some embodiments, the paclitaxel is administered at an amount of about 250 mg to about 350 mg in combination with Compound A and a checkpoint inhibitor at least 5 times a week.

In some embodiments, the paclitaxel is administered at an amount of about 250 mg to about 340 mg in combination with Compound A and a checkpoint inhibitor at least 5 times a week.

In some embodiments, the paclitaxel is administered at an amount of about 250 mg to about 330 mg in combination with Compound A and a checkpoint inhibitor at least 5 times a week.

In some embodiments, the paclitaxel is administered at an amount of about 250 mg to about 320 mg in combination with Compound A and a checkpoint inhibitor at least 5 times a week.

In some embodiments, the paclitaxel is administered at an amount of about 250 mg to about 310 mg in combination with Compound A and a checkpoint inhibitor at least 5 times a week.

In some embodiments, the paclitaxel is administered at an amount of about 250 mg to about 300 mg in combination with Compound A and a checkpoint inhibitor at least 5 times a week.

In some embodiments, the paclitaxel is administered at an amount of about 250 mg to about 290 mg in combination with Compound A and a checkpoint inhibitor at least 5 times a week.

In some embodiments, the paclitaxel is administered at an amount of about 250 mg to about 280 mg in combination with Compound A and a checkpoint inhibitor at least 5 times a week.

In some embodiments, the paclitaxel is administered at an amount of about 250 mg to about 270 mg in combination with Compound A and a checkpoint inhibitor at least 5 times a week.

In some embodiments, the paclitaxel is administered at an amount of about 250 mg to about 260 mg in combination with Compound A and a checkpoint inhibitor at least 5 times a week.

In some embodiments, the paclitaxel is administered at an amount of about 100 mg in combination with Compound A and a checkpoint inhibitor at least 5 times a week.

In some embodiments, the paclitaxel is administered at an amount of about 105 mg in combination with Compound A and a checkpoint inhibitor at least 5 times a week.

In some embodiments, the paclitaxel is administered at an amount of about 110 mg in combination with Compound A and a checkpoint inhibitor at least 5 times a week.

In some embodiments, the paclitaxel is administered at an amount of about 115 mg in combination with Compound A and a checkpoint inhibitor at least 5 times a week.

In some embodiments, the paclitaxel is administered at an amount of about 120 mg in combination with Compound A and a checkpoint inhibitor at least 5 times a week.

In some embodiments, the paclitaxel is administered at an amount of about 125 mg in combination with Compound A and a checkpoint inhibitor at least 5 times a week.

In some embodiments, the paclitaxel is administered at an amount of about 130 mg in combination with Compound A and a checkpoint inhibitor at least 5 times a week.

In some embodiments, the paclitaxel is administered at an amount of about 135 mg in combination with Compound A and a checkpoint inhibitor at least 5 times a week.

In some embodiments, the paclitaxel is administered at an amount of about 140 mg in combination with Compound A and a checkpoint inhibitor at least 5 times a week.

In some embodiments, the paclitaxel is administered at an amount of about 145 mg in combination with Compound A and a checkpoint inhibitor at least 5 times a week.

In some embodiments, the paclitaxel is administered at an amount of about 150 mg in combination with Compound A and a checkpoint inhibitor at least 5 times a week.

In some embodiments, the paclitaxel is administered at an amount of about 155 mg in combination with Compound A and a checkpoint inhibitor at least 5 times a week.

In some embodiments, the paclitaxel is administered at an amount of about 160 mg in combination with Compound A and a checkpoint inhibitor at least 5 times a week.

In some embodiments, the paclitaxel is administered at an amount of about 165 mg in combination with Compound A and a checkpoint inhibitor at least 5 times a week.

In some embodiments, the paclitaxel is administered at an amount of about 170 mg in combination with Compound A and a checkpoint inhibitor at least 5 times a week.

In some embodiments, the paclitaxel is administered at an amount of about 175 mg in combination with Compound A and a checkpoint inhibitor at least 5 times a week.

In some embodiments, the paclitaxel is administered at an amount of about 180 mg in combination with Compound A and a checkpoint inhibitor at least 5 times a week.

In some embodiments, the paclitaxel is administered at an amount of about 185 mg in combination with Compound A and a checkpoint inhibitor at least 5 times a week.

In some embodiments, the paclitaxel is administered at an amount of about 190 mg in combination with Compound A and a checkpoint inhibitor at least 5 times a week.

In some embodiments, the paclitaxel is administered at an amount of about 195 mg in combination with Compound A and a checkpoint inhibitor at least 5 times a week.

In some embodiments, the paclitaxel is administered at an amount of about 200 mg in combination with Compound A and a checkpoint inhibitor at least 5 times a week.

In some embodiments, the paclitaxel is administered at an amount of about 205 mg in combination with Compound A and a checkpoint inhibitor at least 5 times a week.

In some embodiments, the paclitaxel is administered at an amount of about 210 mg in combination with Compound A and a checkpoint inhibitor at least 5 times a week.

In some embodiments, the paclitaxel is administered at an amount of about 215 mg in combination with Compound A and a checkpoint inhibitor at least 5 times a week.

In some embodiments, the paclitaxel is administered at an amount of about 220 mg in combination with Compound A and a checkpoint inhibitor at least 5 times a week.

In some embodiments, the paclitaxel is administered at an amount of about 225 mg in combination with Compound A and a checkpoint inhibitor at least 5 times a week.

In some embodiments, the paclitaxel is administered at an amount of about 230 mg in combination with Compound A and a checkpoint inhibitor at least 5 times a week.

In some embodiments, the paclitaxel is administered at an amount of about 235 mg in combination with Compound A and a checkpoint inhibitor at least 5 times a week.

In some embodiments, the paclitaxel is administered at an amount of about 240 mg in combination with Compound A and a checkpoint inhibitor at least 5 times a week.

In some embodiments, the paclitaxel is administered at an amount of about 245 mg in combination with Compound A and a checkpoint inhibitor at least 5 times a week.

In some embodiments, the paclitaxel is administered at an amount of about 250 mg in combination with Compound A and a checkpoint inhibitor at least 5 times a week.

In some embodiments, the paclitaxel is administered at an amount of about 255 mg in combination with Compound A and a checkpoint inhibitor at least 5 times a week.

In some embodiments, the paclitaxel is administered at an amount of about 260 mg in combination with Compound A and a checkpoint inhibitor at least 5 times a week.

In some embodiments, the paclitaxel is administered at an amount of about 265 mg in combination with Compound A and a checkpoint inhibitor at least 5 times a week.

In some embodiments, the paclitaxel is administered at an amount of about 270 mg in combination with Compound A and a checkpoint inhibitor at least 5 times a week.

In some embodiments, the paclitaxel is administered at an amount of about 275 mg in combination with Compound A and a checkpoint inhibitor at least 5 times a week.

In some embodiments, the paclitaxel is administered at an amount of about 280 mg in combination with Compound A and a checkpoint inhibitor at least 5 times a week.

In some embodiments, the paclitaxel is administered at an amount of about 285 mg in combination with Compound A and a checkpoint inhibitor at least 5 times a week.

In some embodiments, the paclitaxel is administered at an amount of about 290 mg in combination with Compound A and a checkpoint inhibitor at least 5 times a week.

In some embodiments, the paclitaxel is administered at an amount of about 295 mg in combination with Compound A and a checkpoint inhibitor at least 5 times a week.

In some embodiments, the paclitaxel is administered at an amount of about 300 mg in combination with Compound A and a checkpoint inhibitor at least 5 times a week.

In some embodiments, the paclitaxel is administered at an amount of about 305 mg in combination with Compound A and a checkpoint inhibitor at least 5 times a week.

In some embodiments, the paclitaxel is administered at an amount of about 310 mg in combination with Compound A and a checkpoint inhibitor at least 5 times a week.

In some embodiments, the paclitaxel is administered at an amount of about 315 mg in combination with Compound A and a checkpoint inhibitor at least 5 times a week.

In some embodiments, the paclitaxel is administered at an amount of about 320 mg in combination with Compound A and a checkpoint inhibitor at least 5 times a week.

In some embodiments, the paclitaxel is administered at an amount of about 325 mg in combination with Compound A and a checkpoint inhibitor at least 5 times a week.

In some embodiments, the paclitaxel is administered at an amount of about 330 mg in combination with Compound A and a checkpoint inhibitor at least 5 times a week.

In some embodiments, the paclitaxel is administered at an amount of about 335 mg in combination with Compound A and a checkpoint inhibitor at least 5 times a week.

In some embodiments, the paclitaxel is administered at an amount of about 340 mg in combination with Compound A and a checkpoint inhibitor at least 5 times a week.

In some embodiments, the paclitaxel is administered at an amount of about 345 mg in combination with Compound A and a checkpoint inhibitor at least 5 times a week.

In some embodiments, the paclitaxel is administered at an amount of about 350 mg in combination with Compound A and a checkpoint inhibitor at least 5 times a week.

In some embodiments, the paclitaxel is administered at an amount of about 355 mg in combination with Compound A and a checkpoint inhibitor at least 5 times a week.

In some embodiments, the paclitaxel is administered at an amount of about 360 mg in combination with Compound A and a checkpoint inhibitor at least 5 times a week.

In some embodiments, the paclitaxel is administered at an amount of about 365 mg in combination with Compound A and a checkpoint inhibitor at least 5 times a week.

In some embodiments, the paclitaxel is administered at an amount of about 370 mg in combination with Compound A and a checkpoint inhibitor at least 5 times a week.

In some embodiments, the paclitaxel is administered at an amount of about 375 mg in combination with Compound A and a checkpoint inhibitor at least 5 times a week.

In some embodiments, the paclitaxel is administered at an amount of about 380 mg in combination with Compound A and a checkpoint inhibitor at least 5 times a week.

In some embodiments, the paclitaxel is administered at an amount of about 385 mg in combination with Compound A and a checkpoint inhibitor at least 5 times a week.

In some embodiments, the paclitaxel is administered at an amount of about 390 mg in combination with Compound A and a checkpoint inhibitor at least 5 times a week.

In some embodiments, the paclitaxel is administered at an amount of about 395 mg in combination with Compound A and a checkpoint inhibitor at least 5 times a week.

In some embodiments, the paclitaxel is administered at an amount of about 400 mg in combination with Compound A and a checkpoint inhibitor at least 5 times a week.

In some embodiments, the paclitaxel is administered in combination with Compound A and a checkpoint inhibitor at least 5 times a week, wherein the at least 5 days are consecutive.

In some embodiments, the paclitaxel is administered in combination with Compound A and a checkpoint inhibitor at least 6 times a week.

In some embodiments, the paclitaxel is administered at an amount of about 100 mg to about 400 mg in combination with Compound A and a checkpoint inhibitor at least 6 times a week.

In some embodiments, the paclitaxel is administered at an amount of about 100 mg to about 350 mg in combination with Compound A and a checkpoint inhibitor at least 6 times a week.

In some embodiments, the paclitaxel is administered at an amount of about 100 mg to about 300 mg in combination with Compound A and a checkpoint inhibitor at least 6 times a week.

In some embodiments, the paclitaxel is administered at an amount of about 150 mg to about 400 mg in combination with Compound A and a checkpoint inhibitor at least 6 times a week.

In some embodiments, the paclitaxel is administered at an amount of about 150 mg to about 350 mg in combination with Compound A and a checkpoint inhibitor at least 6 times a week.

In some embodiments, the paclitaxel is administered at an amount of about 150 mg to about 340 mg in combination with Compound A and a checkpoint inhibitor at least 6 times a week.

In some embodiments, the paclitaxel is administered at an amount of about 150 mg to about 330 mg in combination with Compound A and a checkpoint inhibitor at least 6 times a week.

In some embodiments, the paclitaxel is administered at an amount of about 150 mg to about 320 mg in combination with Compound A and a checkpoint inhibitor at least 6 times a week.

In some embodiments, the paclitaxel is administered at an amount of about 150 mg to about 310 mg in combination with Compound A and a checkpoint inhibitor at least 6 times a week.

In some embodiments, the paclitaxel is administered at an amount of about 150 mg to about 300 mg in combination with Compound A and a checkpoint inhibitor at least 6 times a week.

In some embodiments, the paclitaxel is administered at an amount of about 150 mg to about 290 mg in combination with Compound A and a checkpoint inhibitor at least 6 times a week. 8n some embodiments, the paclitaxel is administered at an amount of about 150 mg to about 280 mg in combination with Compound A and a checkpoint inhibitor at least 6 times a week.

In some embodiments, the paclitaxel is administered at an amount of about 150 mg to about 270 mg in combination with Compound A and a checkpoint inhibitor at least 6 times a week.

In some embodiments, the paclitaxel is administered at an amount of about 150 mg to about 260 mg in combination with Compound A and a checkpoint inhibitor at least 6 times a week.

In some embodiments, the paclitaxel is administered at an amount of about 150 mg to about 250 mg in combination with Compound A and a checkpoint inhibitor at least 6 times a week.

In some embodiments, the paclitaxel is administered at an amount of about 200 mg to about 400 mg in combination with Compound A and a checkpoint inhibitor at least 6 times a week.

In some embodiments, the paclitaxel is administered at an amount of about 200 mg to about 350 mg in combination with Compound A and a checkpoint inhibitor at least 6 times a week.

In some embodiments, the paclitaxel is administered at an amount of about 200 mg to about 340 mg in combination with Compound A and a checkpoint inhibitor at least 6 times a week.

In some embodiments, the paclitaxel is administered at an amount of about 200 mg to about 330 mg in combination with Compound A and a checkpoint inhibitor at least 6 times a week.

In some embodiments, the paclitaxel is administered at an amount of about 200 mg to about 320 mg in combination with Compound A and a checkpoint inhibitor at least 6 times a week.

In some embodiments, the paclitaxel is administered at an amount of about 200 mg to about 310 mg in combination with Compound A and a checkpoint inhibitor at least 6 times a week.

In some embodiments, the paclitaxel is administered at an amount of about 200 mg to about 300 mg in combination with Compound A and a checkpoint inhibitor at least 6 times a week.

In some embodiments, the paclitaxel is administered at an amount of about 200 mg to about 295 mg in combination with Compound A and a checkpoint inhibitor at least 6 times a week.

In some embodiments, the paclitaxel is administered at an amount of about 200 mg to about 290 mg in combination with Compound A and a checkpoint inhibitor at least 6 times a week.

In some embodiments, the paclitaxel is administered at an amount of about 200 mg to about 285 mg in combination with Compound A and a checkpoint inhibitor at least 6 times a week.

In some embodiments, the paclitaxel is administered at an amount of about 200 mg to about 280 mg in combination with Compound A and a checkpoint inhibitor at least 6 times a week.

In some embodiments, the paclitaxel is administered at an amount of about 200 mg to about 275 mg in combination with Compound A and a checkpoint inhibitor at least 6 times a week.

In some embodiments, the paclitaxel is administered at an amount of about 200 mg to about 270 mg in combination with Compound A and a checkpoint inhibitor at least 6 times a week.

In some embodiments, the paclitaxel is administered at an amount of about 200 mg to about 265 mg in combination with Compound A and a checkpoint inhibitor at least 6 times a week.

In some embodiments, the paclitaxel is administered at an amount of about 200 mg to about 260 mg in combination with Compound A and a checkpoint inhibitor at least 6 times a week.

In some embodiments, the paclitaxel is administered at an amount of about 200 mg to about 255 mg in combination with Compound A and a checkpoint inhibitor at least 6 times a week.

In some embodiments, the paclitaxel is administered at an amount of about 200 mg to about 250 mg in combination with Compound A and a checkpoint inhibitor at least 6 times a week.

In some embodiments, the paclitaxel is administered at an amount of about 250 mg to about 400 mg in combination with Compound A and a checkpoint inhibitor at least 6 times a week.

In some embodiments, the paclitaxel is administered at an amount of about 250 mg to about 350 mg in combination with Compound A and a checkpoint inhibitor at least 6 times a week.

In some embodiments, the paclitaxel is administered at an amount of about 250 mg to about 340 mg in combination with Compound A and a checkpoint inhibitor at least 6 times a week.

In some embodiments, the paclitaxel is administered at an amount of about 250 mg to about 330 mg in combination with Compound A and a checkpoint inhibitor at least 6 times a week.

In some embodiments, the paclitaxel is administered at an amount of about 250 mg to about 320 mg in combination with Compound A and a checkpoint inhibitor at least 6 times a week.

In some embodiments, the paclitaxel is administered at an amount of about 250 mg to about 310 mg in combination with Compound A and a checkpoint inhibitor at least 6 times a week.

In some embodiments, the paclitaxel is administered at an amount of about 250 mg to about 300 mg in combination with Compound A and a checkpoint inhibitor at least 6 times a week.

In some embodiments, the paclitaxel is administered at an amount of about 250 mg to about 290 mg in combination with Compound A and a checkpoint inhibitor at least 6 times a week.

In some embodiments, the paclitaxel is administered at an amount of about 250 mg to about 280 mg in combination with Compound A and a checkpoint inhibitor at least 6 times a week.

In some embodiments, the paclitaxel is administered at an amount of about 250 mg to about 270 mg in combination with Compound A and a checkpoint inhibitor at least 6 times a week.

In some embodiments, the paclitaxel is administered at an amount of about 250 mg to about 260 mg in combination with Compound A and a checkpoint inhibitor at least 6 times a week.

In some embodiments, the paclitaxel is administered at an amount of about 100 mg in combination with Compound A and a checkpoint inhibitor at least 6 times a week.

In some embodiments, the paclitaxel is administered at an amount of about 105 mg in combination with Compound A and a checkpoint inhibitor at least 6 times a week.

In some embodiments, the paclitaxel is administered at an amount of about 110 mg in combination with Compound A and a checkpoint inhibitor at least 6 times a week.

In some embodiments, the paclitaxel is administered at an amount of about 115 mg in combination with Compound A and a checkpoint inhibitor at least 6 times a week.

In some embodiments, the paclitaxel is administered at an amount of about 120 mg in combination with Compound A and a checkpoint inhibitor at least 6 times a week.

In some embodiments, the paclitaxel is administered at an amount of about 125 mg in combination with Compound A and a checkpoint inhibitor at least 6 times a week.

In some embodiments, the paclitaxel is administered at an amount of about 130 mg in combination with Compound A and a checkpoint inhibitor at least 6 times a week.

In some embodiments, the paclitaxel is administered at an amount of about 135 mg in combination with Compound A and a checkpoint inhibitor at least 6 times a week.

In some embodiments, the paclitaxel is administered at an amount of about 140 mg in combination with Compound A and a checkpoint inhibitor at least 6 times a week.

In some embodiments, the paclitaxel is administered at an amount of about 145 mg in combination with Compound A and a checkpoint inhibitor at least 6 times a week.

In some embodiments, the paclitaxel is administered at an amount of about 150 mg in combination with Compound A and a checkpoint inhibitor at least 6 times a week.

In some embodiments, the paclitaxel is administered at an amount of about 155 mg in combination with Compound A and a checkpoint inhibitor at least 6 times a week.

In some embodiments, the paclitaxel is administered at an amount of about 160 mg in combination with Compound A and a checkpoint inhibitor at least 6 times a week.

In some embodiments, the paclitaxel is administered at an amount of about 165 mg in combination with Compound A and a checkpoint inhibitor at least 6 times a week.

In some embodiments, the paclitaxel is administered at an amount of about 170 mg in combination with Compound A and a checkpoint inhibitor at least 6 times a week.

In some embodiments, the paclitaxel is administered at an amount of about 175 mg in combination with Compound A and a checkpoint inhibitor at least 6 times a week.

In some embodiments, the paclitaxel is administered at an amount of about 180 mg in combination with Compound A and a checkpoint inhibitor at least 6 times a week.

In some embodiments, the paclitaxel is administered at an amount of about 185 mg in combination with Compound A and a checkpoint inhibitor at least 6 times a week.

In some embodiments, the paclitaxel is administered at an amount of about 190 mg in combination with Compound A and a checkpoint inhibitor at least 6 times a week.

In some embodiments, the paclitaxel is administered at an amount of about 195 mg in combination with Compound A and a checkpoint inhibitor at least 6 times a week.

In some embodiments, the paclitaxel is administered at an amount of about 200 mg in combination with Compound A and a checkpoint inhibitor at least 6 times a week.

In some embodiments, the paclitaxel is administered at an amount of about 205 mg in combination with Compound A and a checkpoint inhibitor at least 6 times a week.

In some embodiments, the paclitaxel is administered at an amount of about 210 mg in combination with Compound A and a checkpoint inhibitor at least 6 times a week.

In some embodiments, the paclitaxel is administered at an amount of about 215 mg in combination with Compound A and a checkpoint inhibitor at least 6 times a week.

In some embodiments, the paclitaxel is administered at an amount of about 220 mg in combination with Compound A and a checkpoint inhibitor at least 6 times a week.

In some embodiments, the paclitaxel is administered at an amount of about 225 mg in combination with Compound A and a checkpoint inhibitor at least 6 times a week.

In some embodiments, the paclitaxel is administered at an amount of about 230 mg in combination with Compound A and a checkpoint inhibitor at least 6 times a week.

In some embodiments, the paclitaxel is administered at an amount of about 235 mg in combination with Compound A and a checkpoint inhibitor at least 6 times a week.

In some embodiments, the paclitaxel is administered at an amount of about 240 mg in combination with Compound A and a checkpoint inhibitor at least 6 times a week.

In some embodiments, the paclitaxel is administered at an amount of about 245 mg in combination with Compound A and a checkpoint inhibitor at least 6 times a week.

In some embodiments, the paclitaxel is administered at an amount of about 250 mg in combination with Compound A and a checkpoint inhibitor at least 6 times a week.

In some embodiments, the paclitaxel is administered at an amount of about 255 mg in combination with Compound A and a checkpoint inhibitor at least 6 times a week.

In some embodiments, the paclitaxel is administered at an amount of about 260 mg in combination with Compound A and a checkpoint inhibitor at least 6 times a week.

In some embodiments, the paclitaxel is administered at an amount of about 265 mg in combination with Compound A and a checkpoint inhibitor at least 6 times a week.

In some embodiments, the paclitaxel is administered at an amount of about 270 mg in combination with Compound A and a checkpoint inhibitor at least 6 times a week.

In some embodiments, the paclitaxel is administered at an amount of about 275 mg in combination with Compound A and a checkpoint inhibitor at least 6 times a week.

In some embodiments, the paclitaxel is administered at an amount of about 280 mg in combination with Compound A and a checkpoint inhibitor at least 6 times a week.

In some embodiments, the paclitaxel is administered at an amount of about 285 mg in combination with Compound A and a checkpoint inhibitor at least 6 times a week.

In some embodiments, the paclitaxel is administered at an amount of about 290 mg in combination with Compound A and a checkpoint inhibitor at least 6 times a week.

In some embodiments, the paclitaxel is administered at an amount of about 295 mg in combination with Compound A and a checkpoint inhibitor at least 6 times a week.

In some embodiments, the paclitaxel is administered at an amount of about 300 mg in combination with Compound A and a checkpoint inhibitor at least 6 times a week.

In some embodiments, the paclitaxel is administered at an amount of about 305 mg in combination with Compound A and a checkpoint inhibitor at least 6 times a week.

In some embodiments, the paclitaxel is administered at an amount of about 310 mg in combination with Compound A and a checkpoint inhibitor at least 6 times a week.

In some embodiments, the paclitaxel is administered at an amount of about 315 mg in combination with Compound A and a checkpoint inhibitor at least 6 times a week.

In some embodiments, the paclitaxel is administered at an amount of about 320 mg in combination with Compound A and a checkpoint inhibitor at least 6 times a week.

In some embodiments, the paclitaxel is administered at an amount of about 325 mg in combination with Compound A and a checkpoint inhibitor at least 6 times a week.

In some embodiments, the paclitaxel is administered at an amount of about 330 mg in combination with Compound A and a checkpoint inhibitor at least 6 times a week.

In some embodiments, the paclitaxel is administered at an amount of about 335 mg in combination with Compound A and a checkpoint inhibitor at least 6 times a week.

In some embodiments, the paclitaxel is administered at an amount of about 340 mg in combination with Compound A and a checkpoint inhibitor at least 6 times a week.

In some embodiments, the paclitaxel is administered at an amount of about 345 mg in combination with Compound A and a checkpoint inhibitor at least 6 times a week.

In some embodiments, the paclitaxel is administered at an amount of about 350 mg in combination with Compound A and a checkpoint inhibitor at least 6 times a week.

In some embodiments, the paclitaxel is administered at an amount of about 355 mg in combination with Compound A and a checkpoint inhibitor at least 6 times a week.

In some embodiments, the paclitaxel is administered at an amount of about 360 mg in combination with Compound A and a checkpoint inhibitor at least 6 times a week.

In some embodiments, the paclitaxel is administered at an amount of about 365 mg in combination with Compound A and a checkpoint inhibitor at least 6 times a week.

In some embodiments, the paclitaxel is administered at an amount of about 370 mg in combination with Compound A and a checkpoint inhibitor at least 6 times a week.

In some embodiments, the paclitaxel is administered at an amount of about 375 mg in combination with Compound A and a checkpoint inhibitor at least 6 times a week.

In some embodiments, the paclitaxel is administered at an amount of about 380 mg in combination with Compound A and a checkpoint inhibitor at least 6 times a week.

In some embodiments, the paclitaxel is administered at an amount of about 385 mg in combination with Compound A and a checkpoint inhibitor at least 6 times a week.

In some embodiments, the paclitaxel is administered at an amount of about 390 mg in combination with Compound A and a checkpoint inhibitor at least 6 times a week.

In some embodiments, the paclitaxel is administered at an amount of about 395 mg in combination with Compound A and a checkpoint inhibitor at least 6 times a week.

In some embodiments, the paclitaxel is administered at an amount of about 400 mg in combination with Compound A and a checkpoint inhibitor at least 6 times a week.

In some embodiments, Compound A is administered at an amount of about 1 mg to about 500 mg in combination with paclitaxel and a checkpoint inhibitor.

In some embodiments, Compound A is administered at about 1 mg to about 400 mg in combination with paclitaxel and a checkpoint inhibitor.

In some embodiments, Compound A is administered at about 1 mg to about 300 mg in combination with paclitaxel and a checkpoint inhibitor.

In some embodiments, Compound A is administered at about 5 mg to about 200 mg in combination with paclitaxel and a checkpoint inhibitor.

In some embodiments, Compound A is administered at about 10 mg to about 100 mg in combination with paclitaxel and a checkpoint inhibitor.

In some embodiments, Compound A is administered at about 10 mg to about 95 mg in combination with paclitaxel and a checkpoint inhibitor.

In some embodiments, Compound A is administered at about 10 mg to about 90 mg in combination with paclitaxel and a checkpoint inhibitor.

In some embodiments, Compound A is administered at about 10 mg to about 85 mg in combination with paclitaxel and a checkpoint inhibitor.

In some embodiments, Compound A is administered at about 10 mg to about 80 mg in combination with paclitaxel and a checkpoint inhibitor.

In some embodiments, Compound A is administered at about 10 mg to about 75 mg in combination with paclitaxel and a checkpoint inhibitor.

In some embodiments, Compound A is administered at about 10 mg to about 70 mg in combination with paclitaxel and a checkpoint inhibitor.

In some embodiments, Compound A is administered at about 10 mg to about 65 mg in combination with paclitaxel and a checkpoint inhibitor.

In some embodiments, Compound A is administered at about 10 mg to about 60 mg in combination with paclitaxel and a checkpoint inhibitor.

In some embodiments, Compound A is administered at about 10 mg to about 55 mg in combination with paclitaxel and a checkpoint inhibitor.

In some embodiments, Compound A is administered at about 10 mg to about 50 mg in combination with paclitaxel and a checkpoint inhibitor.

In some embodiments, Compound A is administered at about 10 mg to about 45 mg in combination with paclitaxel and a checkpoint inhibitor.

In some embodiments, Compound A is administered at about 10 mg to about 40 mg in combination with paclitaxel and a checkpoint inhibitor.

In some embodiments, Compound A is administered at about 10 mg to about 35 mg in combination with paclitaxel and a checkpoint inhibitor.

In some embodiments, Compound A is administered at about 10 mg to about 30 mg in combination with paclitaxel and a checkpoint inhibitor.

In some embodiments, Compound A is administered at about 10 mg to about 25 mg in combination with paclitaxel and a checkpoint inhibitor.

In some embodiments, Compound A is administered at about 10 mg to about 20 mg in combination with paclitaxel and a checkpoint inhibitor.

In some embodiments, Compound A is administered at about 10 mg to about 15 mg in combination with paclitaxel and a checkpoint inhibitor.

In some embodiments, Compound A is administered at about 15 mg to about 50 mg in combination with paclitaxel and a checkpoint inhibitor.

In some embodiments, Compound A is administered at about 15 mg to about 45 mg in combination with paclitaxel and a checkpoint inhibitor.

In some embodiments, Compound A is administered at about 15 mg to about 40 mg in combination with paclitaxel and a checkpoint inhibitor.

In some embodiments, Compound A is administered at about 15 mg to about 35 mg in combination with paclitaxel and a checkpoint inhibitor.

In some embodiments, Compound A is administered at about 15 mg to about 30 mg in combination with paclitaxel and a checkpoint inhibitor.

In some embodiments, Compound A is administered at about 15 mg to about 25 mg in combination with paclitaxel and a checkpoint inhibitor.

In some embodiments, Compound A is administered at about 15 mg to about 20 mg in combination with paclitaxel and a checkpoint inhibitor.

In some embodiments, Compound A is administered at about 5 mg in combination with paclitaxel and a checkpoint inhibitor.

In some embodiments, Compound A is administered at about 10 mg in combination with paclitaxel and a checkpoint inhibitor.

In some embodiments, Compound A is administered at about 15 mg in combination with paclitaxel and a checkpoint inhibitor.

In some embodiments, Compound A is administered at about 20 mg in combination with paclitaxel and a checkpoint inhibitor.

In some embodiments, Compound A is administered at about 25 mg in combination with paclitaxel and a checkpoint inhibitor.

In some embodiments, Compound A is administered at about 30 mg in combination with paclitaxel and a checkpoint inhibitor.

In some embodiments, Compound A is administered at about 35 mg in combination with paclitaxel and a checkpoint inhibitor.

In some embodiments, Compound A is administered at about 40 mg in combination with paclitaxel and a checkpoint inhibitor.

In some embodiments, Compound A is administered at about 45 mg in combination with paclitaxel and a checkpoint inhibitor.

In some embodiments, Compound A is administered at about 50 mg in combination with paclitaxel and a checkpoint inhibitor.

In some embodiments, Compound A is administered in combination with paclitaxel and a checkpoint inhibitor at least 1 time a week.

In some embodiments, Compound A is administered at an amount of about 1 mg to about 500 mg in combination with paclitaxel and a checkpoint inhibitor at least 1 time a week.

In some embodiments, Compound A is administered at about 1 mg to about 400 mg in combination with paclitaxel and a checkpoint inhibitor at least 1 time a week.

In some embodiments, Compound A is administered at about 1 mg to about 300 mg in combination with paclitaxel and a checkpoint inhibitor at least 1 time a week.

In some embodiments, Compound A is administered at about 5 mg to about 200 mg in combination with paclitaxel and a checkpoint inhibitor at least 1 time a week.

In some embodiments, Compound A is administered at about 10 mg to about 100 mg in combination with paclitaxel and a checkpoint inhibitor at least 1 time a week.

In some embodiments, Compound A is administered at about 10 mg to about 95 mg in combination with paclitaxel and a checkpoint inhibitor at least 1 time a week.

In some embodiments, Compound A is administered at about 10 mg to about 90 mg in combination with paclitaxel and a checkpoint inhibitor at least 1 time a week.

In some embodiments, Compound A is administered at about 10 mg to about 85 mg in combination with paclitaxel and a checkpoint inhibitor at least 1 time a week.

In some embodiments, Compound A is administered at about 10 mg to about 80 mg in combination with paclitaxel and a checkpoint inhibitor at least 1 time a week.

In some embodiments, Compound A is administered at about 10 mg to about 75 mg in combination with paclitaxel and a checkpoint inhibitor at least 1 time a week.

In some embodiments, Compound A is administered at about 10 mg to about 70 mg in combination with paclitaxel and a checkpoint inhibitor at least 1 time a week.

In some embodiments, Compound A is administered at about 10 mg to about 65 mg in combination with paclitaxel and a checkpoint inhibitor at least 1 time a week.

In some embodiments, Compound A is administered at about 10 mg to about 60 mg in combination with paclitaxel and a checkpoint inhibitor at least 1 time a week.

In some embodiments, Compound A is administered at about 10 mg to about 55 mg in combination with paclitaxel and a checkpoint inhibitor at least 1 time a week.

In some embodiments, Compound A is administered at about 10 mg to about 50 mg in combination with paclitaxel and a checkpoint inhibitor at least 1 time a week.

In some embodiments, Compound A is administered at about 10 mg to about 45 mg in combination with paclitaxel and a checkpoint inhibitor at least 1 time a week.

In some embodiments, Compound A is administered at about 10 mg to about 40 mg in combination with paclitaxel and a checkpoint inhibitor at least 1 time a week.

In some embodiments, Compound A is administered at about 10 mg to about 35 mg in combination with paclitaxel and a checkpoint inhibitor at least 1 time a week.

In some embodiments, Compound A is administered at about 10 mg to about 30 mg in combination with paclitaxel and a checkpoint inhibitor at least 1 time a week.

In some embodiments, Compound A is administered at about 10 mg to about 25 mg in combination with paclitaxel and a checkpoint inhibitor at least 1 time a week.

In some embodiments, Compound A is administered at about 10 mg to about 20 mg in combination with paclitaxel and a checkpoint inhibitor at least 1 time a week.

In some embodiments, Compound A is administered at about 10 mg to about 15 mg in combination with paclitaxel and a checkpoint inhibitor at least 1 time a week.

In some embodiments, Compound A is administered at about 15 mg to about 50 mg in combination with paclitaxel and a checkpoint inhibitor at least 1 time a week.

In some embodiments, Compound A is administered at about 15 mg to about 45 mg in combination with paclitaxel and a checkpoint inhibitor at least 1 time a week.

In some embodiments, Compound A is administered at about 15 mg to about 40 mg in combination with paclitaxel and a checkpoint inhibitor at least 1 time a week.

In some embodiments, Compound A is administered at about 15 mg to about 35 mg in combination with paclitaxel and a checkpoint inhibitor at least 1 time a week.

In some embodiments, Compound A is administered at about 15 mg to about 30 mg in combination with paclitaxel and a checkpoint inhibitor at least 1 time a week.

In some embodiments, Compound A is administered at about 15 mg to about 25 mg in combination with paclitaxel and a checkpoint inhibitor at least 1 time a week.

In some embodiments, Compound A is administered at about 15 mg to about 20 mg in combination with paclitaxel and a checkpoint inhibitor at least 1 time a week.

In some embodiments, Compound A is administered at about 5 mg in combination with paclitaxel and a checkpoint inhibitor at least 1 time a week.

In some embodiments, Compound A is administered at about 10 mg in combination with paclitaxel and a checkpoint inhibitor at least 1 time a week.

In some embodiments, Compound A is administered at about 15 mg in combination with paclitaxel and a checkpoint inhibitor at least 1 time a week.

In some embodiments, Compound A is administered at about 20 mg in combination with paclitaxel and a checkpoint inhibitor at least 1 time a week.

In some embodiments, Compound A is administered at about 25 mg in combination with paclitaxel and a checkpoint inhibitor at least 1 time a week.

In some embodiments, Compound A is administered at about 30 mg in combination with paclitaxel and a checkpoint inhibitor at least 1 time a week.

In some embodiments, Compound A is administered at about 35 mg in combination with paclitaxel and a checkpoint inhibitor at least 1 time a week.

In some embodiments, Compound A is administered at about 40 mg in combination with paclitaxel and a checkpoint inhibitor at least 1 time a week.

In some embodiments, Compound A is administered at about 45 mg in combination with paclitaxel and a checkpoint inhibitor at least 1 time a week.

In some embodiments, Compound A is administered at about 50 mg in combination with paclitaxel and a checkpoint inhibitor at least 1 time a week.

In some embodiments, Compound A is administered in combination with paclitaxel and a checkpoint inhibitor at least 2 times a week.

In some embodiments, Compound A is administered at an amount of about 1 mg to about 500 mg in combination with paclitaxel and a checkpoint inhibitor at least 2 times a week.

In some embodiments, Compound A is administered at about 1 mg to about 400 mg in combination with paclitaxel and a checkpoint inhibitor at least 2 times a week.

In some embodiments, Compound A is administered at about 1 mg to about 300 mg in combination with paclitaxel and a checkpoint inhibitor at least 2 times a week.

In some embodiments, Compound A is administered at about 5 mg to about 200 mg in combination with paclitaxel and a checkpoint inhibitor at least 2 times a week.

In some embodiments, Compound A is administered at about 10 mg to about 100 mg in combination with paclitaxel and a checkpoint inhibitor at least 2 times a week.

In some embodiments, Compound A is administered at about 10 mg to about 95 mg in combination with paclitaxel and a checkpoint inhibitor at least 2 times a week.

In some embodiments, Compound A is administered at about 10 mg to about 90 mg in combination with paclitaxel and a checkpoint inhibitor at least 2 times a week.

In some embodiments, Compound A is administered at about 10 mg to about 85 mg in combination with paclitaxel and a checkpoint inhibitor at least 2 times a week.

In some embodiments, Compound A is administered at about 10 mg to about 80 mg in combination with paclitaxel and a checkpoint inhibitor at least 2 times a week.

In some embodiments, Compound A is administered at about 10 mg to about 75 mg in combination with paclitaxel and a checkpoint inhibitor at least 2 times a week.

In some embodiments, Compound A is administered at about 10 mg to about 70 mg in combination with paclitaxel and a checkpoint inhibitor at least 2 times a week.

In some embodiments, Compound A is administered at about 10 mg to about 65 mg in combination with paclitaxel and a checkpoint inhibitor at least 2 times a week.

In some embodiments, Compound A is administered at about 10 mg to about 60 mg in combination with paclitaxel and a checkpoint inhibitor at least 2 times a week.

In some embodiments, Compound A is administered at about 10 mg to about 55 mg in combination with paclitaxel and a checkpoint inhibitor at least 2 times a week.

In some embodiments, Compound A is administered at about 10 mg to about 50 mg in combination with paclitaxel and a checkpoint inhibitor at least 2 times a week.

In some embodiments, Compound A is administered at about 10 mg to about 45 mg in combination with paclitaxel and a checkpoint inhibitor at least 2 times a week.

In some embodiments, Compound A is administered at about 10 mg to about 40 mg in combination with paclitaxel and a checkpoint inhibitor at least 2 times a week.

In some embodiments, Compound A is administered at about 10 mg to about 35 mg in combination with paclitaxel and a checkpoint inhibitor at least 2 times a week.

In some embodiments, Compound A is administered at about 10 mg to about 30 mg in combination with paclitaxel and a checkpoint inhibitor at least 2 times a week.

In some embodiments, Compound A is administered at about 10 mg to about 25 mg in combination with paclitaxel and a checkpoint inhibitor at least 2 times a week.

In some embodiments, Compound A is administered at about 10 mg to about 20 mg in combination with paclitaxel and a checkpoint inhibitor at least 2 times a week.

In some embodiments, Compound A is administered at about 10 mg to about 15 mg in combination with paclitaxel and a checkpoint inhibitor at least 2 times a week.

In some embodiments, Compound A is administered at about 15 mg to about 50 mg in combination with paclitaxel and a checkpoint inhibitor at least 2 times a week.

In some embodiments, Compound A is administered at about 15 mg to about 45 mg in combination with paclitaxel and a checkpoint inhibitor at least 2 times a week.

In some embodiments, Compound A is administered at about 15 mg to about 40 mg in combination with paclitaxel and a checkpoint inhibitor at least 2 times a week.

In some embodiments, Compound A is administered at about 15 mg to about 35 mg in combination with paclitaxel and a checkpoint inhibitor at least 2 times a week.

In some embodiments, Compound A is administered at about 15 mg to about 30 mg in combination with paclitaxel and a checkpoint inhibitor at least 2 times a week.

In some embodiments, Compound A is administered at about 15 mg to about 25 mg in combination with paclitaxel and a checkpoint inhibitor at least 2 times a week.

In some embodiments, Compound A is administered at about 15 mg to about 20 mg in combination with paclitaxel and a checkpoint inhibitor at least 2 times a week.

In some embodiments, Compound A is administered at about 5 mg in combination with paclitaxel and a checkpoint inhibitor at least 2 times a week.

In some embodiments, Compound A is administered at about 10 mg in combination with paclitaxel and a checkpoint inhibitor at least 2 times a week.

In some embodiments, Compound A is administered at about 15 mg in combination with paclitaxel and a checkpoint inhibitor at least 2 times a week.

In some embodiments, Compound A is administered at about 20 mg in combination with paclitaxel and a checkpoint inhibitor at least 2 times a week.

In some embodiments, Compound A is administered at about 25 mg in combination with paclitaxel and a checkpoint inhibitor at least 2 times a week.

In some embodiments, Compound A is administered at about 30 mg in combination with paclitaxel and a checkpoint inhibitor at least 2 times a week.

In some embodiments, Compound A is administered at about 35 mg in combination with paclitaxel and a checkpoint inhibitor at least 2 times a week.

In some embodiments, Compound A is administered at about 40 mg in combination with paclitaxel and a checkpoint inhibitor at least 2 times a week.

In some embodiments, Compound A is administered at about 45 mg in combination with paclitaxel and a checkpoint inhibitor at least 2 times a week.

In some embodiments, Compound A is administered at about 50 mg in combination with paclitaxel and a checkpoint inhibitor at least 2 times a week.

In some embodiments, Compound A is administered in combination with paclitaxel and a checkpoint inhibitor at least 3 times a week.

In some embodiments, Compound A is administered at an amount of about 1 mg to about 500 mg in combination with paclitaxel and a checkpoint inhibitor at least 3 times a week.

In some embodiments, Compound A is administered at about 1 mg to about 400 mg in combination with paclitaxel and a checkpoint inhibitor at least 3 times a week.

In some embodiments, Compound A is administered at about 1 mg to about 300 mg in combination with paclitaxel and a checkpoint inhibitor at least 3 times a week.

In some embodiments, Compound A is administered at about 5 mg to about 200 mg in combination with paclitaxel and a checkpoint inhibitor at least 3 times a week.

In some embodiments, Compound A is administered at about 10 mg to about 100 mg in combination with paclitaxel and a checkpoint inhibitor at least 3 times a week.

In some embodiments, Compound A is administered at about 10 mg to about 95 mg in combination with paclitaxel and a checkpoint inhibitor at least 3 times a week.

In some embodiments, Compound A is administered at about 10 mg to about 90 mg in combination with paclitaxel and a checkpoint inhibitor at least 3 times a week.

In some embodiments, Compound A is administered at about 10 mg to about 85 mg in combination with paclitaxel and a checkpoint inhibitor at least 3 times a week.

In some embodiments, Compound A is administered at about 10 mg to about 80 mg in combination with paclitaxel and a checkpoint inhibitor at least 3 times a week.

In some embodiments, Compound A is administered at about 10 mg to about 75 mg in combination with paclitaxel and a checkpoint inhibitor at least 3 times a week.

In some embodiments, Compound A is administered at about 10 mg to about 70 mg in combination with paclitaxel and a checkpoint inhibitor at least 3 times a week.

In some embodiments, Compound A is administered at about 10 mg to about 65 mg in combination with paclitaxel and a checkpoint inhibitor at least 3 times a week.

In some embodiments, Compound A is administered at about 10 mg to about 60 mg in combination with paclitaxel and a checkpoint inhibitor at least 3 times a week.

In some embodiments, Compound A is administered at about 10 mg to about 55 mg in combination with paclitaxel and a checkpoint inhibitor at least 3 times a week.

In some embodiments, Compound A is administered at about 10 mg to about 50 mg in combination with paclitaxel and a checkpoint inhibitor at least 3 times a week.

In some embodiments, Compound A is administered at about 10 mg to about 45 mg in combination with paclitaxel and a checkpoint inhibitor at least 3 times a week.

In some embodiments, Compound A is administered at about 10 mg to about 40 mg in combination with paclitaxel and a checkpoint inhibitor at least 3 times a week.

In some embodiments, Compound A is administered at about 10 mg to about 35 mg in combination with paclitaxel and a checkpoint inhibitor at least 3 times a week.

In some embodiments, Compound A is administered at about 10 mg to about 30 mg in combination with paclitaxel and a checkpoint inhibitor at least 3 times a week.

In some embodiments, Compound A is administered at about 10 mg to about 25 mg in combination with paclitaxel and a checkpoint inhibitor at least 3 times a week.

In some embodiments, Compound A is administered at about 10 mg to about 20 mg in combination with paclitaxel and a checkpoint inhibitor at least 3 times a week.

In some embodiments, Compound A is administered at about 10 mg to about 15 mg in combination with paclitaxel and a checkpoint inhibitor at least 3 times a week.

In some embodiments, Compound A is administered at about 15 mg to about 50 mg in combination with paclitaxel and a checkpoint inhibitor at least 3 times a week.

In some embodiments, Compound A is administered at about 15 mg to about 45 mg in combination with paclitaxel and a checkpoint inhibitor at least 3 times a week.

In some embodiments, Compound A is administered at about 15 mg to about 40 mg in combination with paclitaxel and a checkpoint inhibitor at least 3 times a week.

In some embodiments, Compound A is administered at about 15 mg to about 35 mg in combination with paclitaxel and a checkpoint inhibitor at least 3 times a week.

In some embodiments, Compound A is administered at about 15 mg to about 30 mg in combination with paclitaxel and a checkpoint inhibitor at least 3 times a week.

In some embodiments, Compound A is administered at about 15 mg to about 25 mg in combination with paclitaxel and a checkpoint inhibitor at least 3 times a week.

In some embodiments, Compound A is administered at about 15 mg to about 20 mg in combination with paclitaxel and a checkpoint inhibitor at least 3 times a week.

In some embodiments, Compound A is administered at about 5 mg in combination with paclitaxel and a checkpoint inhibitor at least 3 times a week.

In some embodiments, Compound A is administered at about 10 mg in combination with paclitaxel and a checkpoint inhibitor at least 3 times a week.

In some embodiments, Compound A is administered at about 15 mg in combination with paclitaxel and a checkpoint inhibitor at least 3 times a week.

In some embodiments, Compound A is administered at about 20 mg in combination with paclitaxel and a checkpoint inhibitor at least 3 times a week.

In some embodiments, Compound A is administered at about 25 mg in combination with paclitaxel and a checkpoint inhibitor at least 3 times a week.

In some embodiments, Compound A is administered at about 30 mg in combination with paclitaxel and a checkpoint inhibitor at least 3 times a week.

In some embodiments, Compound A is administered at about 35 mg in combination with paclitaxel and a checkpoint inhibitor at least 3 times a week.

In some embodiments, Compound A is administered at about 40 mg in combination with paclitaxel and a checkpoint inhibitor at least 3 times a week.

In some embodiments, Compound A is administered at about 45 mg in combination with paclitaxel and a checkpoint inhibitor at least 3 times a week.

In some embodiments, Compound A is administered at about 50 mg in combination with paclitaxel and a checkpoint inhibitor at least 3 times a week.

In some embodiments, Compound A is administered in combination with paclitaxel and a checkpoint inhibitor at least 4 times a week.

In some embodiments, Compound A is administered at an amount of about 1 mg to about 500 mg in combination with paclitaxel and a checkpoint inhibitor at least 4 times a week.

In some embodiments, Compound A is administered at about 1 mg to about 400 mg in combination with paclitaxel and a checkpoint inhibitor at least 4 times a week.

In some embodiments, Compound A is administered at about 1 mg to about 300 mg in combination with paclitaxel and a checkpoint inhibitor at least 4 times a week.

In some embodiments, Compound A is administered at about 5 mg to about 200 mg in combination with paclitaxel and a checkpoint inhibitor at least 4 times a week.

In some embodiments, Compound A is administered at about 10 mg to about 100 mg in combination with paclitaxel and a checkpoint inhibitor at least 4 times a week.

In some embodiments, Compound A is administered at about 10 mg to about 95 mg in combination with paclitaxel and a checkpoint inhibitor at least 4 times a week.

In some embodiments, Compound A is administered at about 10 mg to about 90 mg in combination with paclitaxel and a checkpoint inhibitor at least 4 times a week.

In some embodiments, Compound A is administered at about 10 mg to about 85 mg in combination with paclitaxel and a checkpoint inhibitor at least 4 times a week.

In some embodiments, Compound A is administered at about 10 mg to about 80 mg in combination with paclitaxel and a checkpoint inhibitor at least 4 times a week.

In some embodiments, Compound A is administered at about 10 mg to about 75 mg in combination with paclitaxel and a checkpoint inhibitor at least 4 times a week.

In some embodiments, Compound A is administered at about 10 mg to about 70 mg in combination with paclitaxel and a checkpoint inhibitor at least 4 times a week.

In some embodiments, Compound A is administered at about 10 mg to about 65 mg in combination with paclitaxel and a checkpoint inhibitor at least 4 times a week.

In some embodiments, Compound A is administered at about 10 mg to about 60 mg in combination with paclitaxel and a checkpoint inhibitor at least 4 times a week.

In some embodiments, Compound A is administered at about 10 mg to about 55 mg in combination with paclitaxel and a checkpoint inhibitor at least 4 times a week.

In some embodiments, Compound A is administered at about 10 mg to about 50 mg in combination with paclitaxel and a checkpoint inhibitor at least 4 times a week.

In some embodiments, Compound A is administered at about 10 mg to about 45 mg in combination with paclitaxel and a checkpoint inhibitor at least 4 times a week.

In some embodiments, Compound A is administered at about 10 mg to about 40 mg in combination with paclitaxel and a checkpoint inhibitor at least 4 times a week.

In some embodiments, Compound A is administered at about 10 mg to about 35 mg in combination with paclitaxel and a checkpoint inhibitor at least 4 times a week.

In some embodiments, Compound A is administered at about 10 mg to about 30 mg in combination with paclitaxel and a checkpoint inhibitor at least 4 times a week.

In some embodiments, Compound A is administered at about 10 mg to about 25 mg in combination with paclitaxel and a checkpoint inhibitor at least 4 times a week.

In some embodiments, Compound A is administered at about 10 mg to about 20 mg in combination with paclitaxel and a checkpoint inhibitor at least 4 times a week.

In some embodiments, Compound A is administered at about 10 mg to about 15 mg in combination with paclitaxel and a checkpoint inhibitor at least 4 times a week.

In some embodiments, Compound A is administered at about 15 mg to about 50 mg in combination with paclitaxel and a checkpoint inhibitor at least 4 times a week.

In some embodiments, Compound A is administered at about 15 mg to about 45 mg in combination with paclitaxel and a checkpoint inhibitor at least 4 times a week.

In some embodiments, Compound A is administered at about 15 mg to about 40 mg in combination with paclitaxel and a checkpoint inhibitor at least 4 times a week.

In some embodiments, Compound A is administered at about 15 mg to about 35 mg in combination with paclitaxel and a checkpoint inhibitor at least 4 times a week.

In some embodiments, Compound A is administered at about 15 mg to about 30 mg in combination with paclitaxel and a checkpoint inhibitor at least 4 times a week.

In some embodiments, Compound A is administered at about 15 mg to about 25 mg in combination with paclitaxel and a checkpoint inhibitor at least 4 times a week.

In some embodiments, Compound A is administered at about 15 mg to about 20 mg in combination with paclitaxel and a checkpoint inhibitor at least 4 times a week.

In some embodiments, Compound A is administered at about 5 mg in combination with paclitaxel and a checkpoint inhibitor at least 4 times a week.

In some embodiments, Compound A is administered at about 10 mg in combination with paclitaxel and a checkpoint inhibitor at least 4 times a week.

In some embodiments, Compound A is administered at about 15 mg in combination with paclitaxel and a checkpoint inhibitor at least 4 times a week.

In some embodiments, Compound A is administered at about 20 mg in combination with paclitaxel and a checkpoint inhibitor at least 4 times a week.

In some embodiments, Compound A is administered at about 25 mg in combination with paclitaxel and a checkpoint inhibitor at least 4 times a week.

In some embodiments, Compound A is administered at about 30 mg in combination with paclitaxel and a checkpoint inhibitor at least 4 times a week.

In some embodiments, Compound A is administered at about 35 mg in combination with paclitaxel and a checkpoint inhibitor at least 4 times a week.

In some embodiments, Compound A is administered at about 40 mg in combination with paclitaxel and a checkpoint inhibitor at least 4 times a week.

In some embodiments, Compound A is administered at about 45 mg in combination with paclitaxel and a checkpoint inhibitor at least 4 times a week.

In some embodiments, Compound A is administered at about 50 mg in combination with paclitaxel and a checkpoint inhibitor at least 4 times a week.

In some embodiments, Compound A is administered in combination with paclitaxel and a checkpoint inhibitor at least 5 times a week.

In some embodiments, Compound A is administered at an amount of about 1 mg to about 500 mg in combination with paclitaxel and a checkpoint inhibitor at least 5 times a week.

In some embodiments, Compound A is administered at about 1 mg to about 400 mg in combination with paclitaxel and a checkpoint inhibitor at least 5 times a week.

In some embodiments, Compound A is administered at about 1 mg to about 300 mg in combination with paclitaxel and a checkpoint inhibitor at least 5 times a week.

In some embodiments, Compound A is administered at about 5 mg to about 200 mg in combination with paclitaxel and a checkpoint inhibitor at least 5 times a week.

In some embodiments, Compound A is administered at about 10 mg to about 100 mg in combination with paclitaxel and a checkpoint inhibitor at least 5 times a week.

In some embodiments, Compound A is administered at about 10 mg to about 95 mg in combination with paclitaxel and a checkpoint inhibitor at least 5 times a week.

In some embodiments, Compound A is administered at about 10 mg to about 90 mg in combination with paclitaxel and a checkpoint inhibitor at least 5 times a week.

In some embodiments, Compound A is administered at about 10 mg to about 85 mg in combination with paclitaxel and a checkpoint inhibitor at least 5 times a week.

In some embodiments, Compound A is administered at about 10 mg to about 80 mg in combination with paclitaxel and a checkpoint inhibitor at least 5 times a week.

In some embodiments, Compound A is administered at about 10 mg to about 75 mg in combination with paclitaxel and a checkpoint inhibitor at least 5 times a week.

In some embodiments, Compound A is administered at about 10 mg to about 70 mg in combination with paclitaxel and a checkpoint inhibitor at least 5 times a week.

In some embodiments, Compound A is administered at about 10 mg to about 65 mg in combination with paclitaxel and a checkpoint inhibitor at least 5 times a week.

In some embodiments, Compound A is administered at about 10 mg to about 60 mg in combination with paclitaxel and a checkpoint inhibitor at least 5 times a week.

In some embodiments, Compound A is administered at about 10 mg to about 55 mg in combination with paclitaxel and a checkpoint inhibitor at least 5 times a week.

In some embodiments, Compound A is administered at about 10 mg to about 50 mg in combination with paclitaxel and a checkpoint inhibitor at least 5 times a week.

In some embodiments, Compound A is administered at about 10 mg to about 45 mg in combination with paclitaxel and a checkpoint inhibitor at least 5 times a week.

In some embodiments, Compound A is administered at about 10 mg to about 40 mg in combination with paclitaxel and a checkpoint inhibitor at least 5 times a week.

In some embodiments, Compound A is administered at about 10 mg to about 35 mg in combination with paclitaxel and a checkpoint inhibitor at least 5 times a week.

In some embodiments, Compound A is administered at about 10 mg to about 30 mg in combination with paclitaxel and a checkpoint inhibitor at least 5 times a week.

In some embodiments, Compound A is administered at about 10 mg to about 25 mg in combination with paclitaxel and a checkpoint inhibitor at least 5 times a week.

In some embodiments, Compound A is administered at about 10 mg to about 20 mg in combination with paclitaxel and a checkpoint inhibitor at least 5 times a week.

In some embodiments, Compound A is administered at about 10 mg to about 15 mg in combination with paclitaxel and a checkpoint inhibitor at least 5 times a week.

In some embodiments, Compound A is administered at about 15 mg to about 50 mg in combination with paclitaxel and a checkpoint inhibitor at least 5 times a week.

In some embodiments, Compound A is administered at about 15 mg to about 45 mg in combination with paclitaxel and a checkpoint inhibitor at least 5 times a week.

In some embodiments, Compound A is administered at about 15 mg to about 40 mg in combination with paclitaxel and a checkpoint inhibitor at least 5 times a week.

In some embodiments, Compound A is administered at about 15 mg to about 35 mg in combination with paclitaxel and a checkpoint inhibitor at least 5 times a week.

In some embodiments, Compound A is administered at about 15 mg to about 30 mg in combination with paclitaxel and a checkpoint inhibitor at least 5 times a week.

In some embodiments, Compound A is administered at about 15 mg to about 25 mg in combination with paclitaxel and a checkpoint inhibitor at least 5 times a week.

In some embodiments, Compound A is administered at about 15 mg to about 20 mg in combination with paclitaxel and a checkpoint inhibitor at least 5 times a week.

In some embodiments, Compound A is administered at about 5 mg in combination with paclitaxel and a checkpoint inhibitor at least 5 times a week.

In some embodiments, Compound A is administered at about 10 mg in combination with paclitaxel and a checkpoint inhibitor at least 5 times a week.

In some embodiments, Compound A is administered at about 15 mg in combination with paclitaxel and a checkpoint inhibitor at least 5 times a week.

In some embodiments, Compound A is administered at about 20 mg in combination with paclitaxel and a checkpoint inhibitor at least 5 times a week.

In some embodiments, Compound A is administered at about 25 mg in combination with paclitaxel and a checkpoint inhibitor at least 5 times a week.

In some embodiments, Compound A is administered at about 30 mg in combination with paclitaxel and a checkpoint inhibitor at least 5 times a week.

In some embodiments, Compound A is administered at about 35 mg in combination with paclitaxel and a checkpoint inhibitor at least 5 times a week.

In some embodiments, Compound A is administered at about 40 mg in combination with paclitaxel and a checkpoint inhibitor at least 5 times a week.

In some embodiments, Compound A is administered at about 45 mg in combination with paclitaxel and a checkpoint inhibitor at least 5 times a week.

In some embodiments, Compound A is administered at about 50 mg in combination with paclitaxel and a checkpoint inhibitor at least 5 times a week.

In some embodiments, Compound A is administered in combination with paclitaxel and a checkpoint inhibitor at least 6 times a week.

In some embodiments, Compound A is administered at an amount of about 1 mg to about 500 mg in combination with paclitaxel and a checkpoint inhibitor at least 6 times a week.

In some embodiments, Compound A is administered at about 1 mg to about 400 mg in combination with paclitaxel and a checkpoint inhibitor at least 6 times a week.

In some embodiments, Compound A is administered at about 1 mg to about 300 mg in combination with paclitaxel and a checkpoint inhibitor at least 6 times a week.

In some embodiments, Compound A is administered at about 5 mg to about 200 mg in combination with paclitaxel and a checkpoint inhibitor at least 6 times a week.

In some embodiments, Compound A is administered at about 10 mg to about 100 mg in combination with paclitaxel and a checkpoint inhibitor at least 6 times a week.

In some embodiments, Compound A is administered at about 10 mg to about 95 mg in combination with paclitaxel and a checkpoint inhibitor at least 6 times a week.

In some embodiments, Compound A is administered at about 10 mg to about 90 mg in combination with paclitaxel and a checkpoint inhibitor at least 6 times a week.

In some embodiments, Compound A is administered at about 10 mg to about 85 mg in combination with paclitaxel and a checkpoint inhibitor at least 6 times a week.

In some embodiments, Compound A is administered at about 10 mg to about 80 mg in combination with paclitaxel and a checkpoint inhibitor at least 6 times a week.

In some embodiments, Compound A is administered at about 10 mg to about 75 mg in combination with paclitaxel and a checkpoint inhibitor at least 6 times a week.

In some embodiments, Compound A is administered at about 10 mg to about 70 mg in combination with paclitaxel and a checkpoint inhibitor at least 6 times a week.

In some embodiments, Compound A is administered at about 10 mg to about 65 mg in combination with paclitaxel and a checkpoint inhibitor at least 6 times a week.

In some embodiments, Compound A is administered at about 10 mg to about 60 mg in combination with paclitaxel and a checkpoint inhibitor at least 6 times a week.

In some embodiments, Compound A is administered at about 10 mg to about 55 mg in combination with paclitaxel and a checkpoint inhibitor at least 6 times a week.

In some embodiments, Compound A is administered at about 10 mg to about 50 mg in combination with paclitaxel and a checkpoint inhibitor at least 6 times a week.

In some embodiments, Compound A is administered at about 10 mg to about 45 mg in combination with paclitaxel and a checkpoint inhibitor at least 6 times a week.

In some embodiments, Compound A is administered at about 10 mg to about 40 mg in combination with paclitaxel and a checkpoint inhibitor at least 6 times a week.

In some embodiments, Compound A is administered at about 10 mg to about 35 mg in combination with paclitaxel and a checkpoint inhibitor at least 6 times a week.

In some embodiments, Compound A is administered at about 10 mg to about 30 mg in combination with paclitaxel and a checkpoint inhibitor at least 6 times a week.

In some embodiments, Compound A is administered at about 10 mg to about 25 mg in combination with paclitaxel and a checkpoint inhibitor at least 6 times a week.

In some embodiments, Compound A is administered at about 10 mg to about 20 mg in combination with paclitaxel and a checkpoint inhibitor at least 6 times a week.

In some embodiments, Compound A is administered at about 10 mg to about 15 mg in combination with paclitaxel and a checkpoint inhibitor at least 6 times a week.

In some embodiments, Compound A is administered at about 15 mg to about 50 mg in combination with paclitaxel and a checkpoint inhibitor at least 6 times a week.

In some embodiments, Compound A is administered at about 15 mg to about 45 mg in combination with paclitaxel and a checkpoint inhibitor at least 6 times a week.

In some embodiments, Compound A is administered at about 15 mg to about 40 mg in combination with paclitaxel and a checkpoint inhibitor at least 6 times a week.

In some embodiments, Compound A is administered at about 15 mg to about 35 mg in combination with paclitaxel and a checkpoint inhibitor at least 6 times a week.

In some embodiments, Compound A is administered at about 15 mg to about 30 mg in combination with paclitaxel and a checkpoint inhibitor at least 6 times a week.

In some embodiments, Compound A is administered at about 15 mg to about 25 mg in combination with paclitaxel and a checkpoint inhibitor at least 6 times a week.

In some embodiments, Compound A is administered at about 15 mg to about 20 mg in combination with paclitaxel and a checkpoint inhibitor at least 6 times a week.

In some embodiments, Compound A is administered at about 5 mg in combination with paclitaxel and a checkpoint inhibitor at least 6 times a week.

In some embodiments, Compound A is administered at about 10 mg in combination with paclitaxel and a checkpoint inhibitor at least 6 times a week.

In some embodiments, Compound A is administered at about 15 mg in combination with paclitaxel and a checkpoint inhibitor at least 6 times a week.

In some embodiments, Compound A is administered at about 20 mg in combination with paclitaxel and a checkpoint inhibitor at least 6 times a week.

In some embodiments, Compound A is administered at about 25 mg in combination with paclitaxel and a checkpoint inhibitor at least 6 times a week.

In some embodiments, Compound A is administered at about 30 mg in combination with paclitaxel and a checkpoint inhibitor at least 6 times a week.

In some embodiments, Compound A is administered at about 35 mg in combination with paclitaxel and a checkpoint inhibitor at least 6 times a week.

In some embodiments, Compound A is administered at about 40 mg in combination with paclitaxel and a checkpoint inhibitor at least 6 times a week.

In some embodiments, Compound A is administered at about 45 mg in combination with paclitaxel and a checkpoint inhibitor at least 6 times a week.

In some embodiments, Compound A is administered at about 50 mg in combination with paclitaxel and a checkpoint inhibitor at least 6 times a week.

In some embodiments, the checkpoint inhibitor is administered at an amount of about 1 mg to about 500 mg in combination with paclitaxel and Compound A.

In some embodiments, the checkpoint inhibitor is administered at about 1 mg to about 400 mg in combination with paclitaxel and Compound A.

In some embodiments, the checkpoint inhibitor is administered at about 1 mg to about 300 mg in combination with paclitaxel and Compound A.

In some embodiments, the checkpoint inhibitor is administered at about 1 mg to about 200 mg in combination with paclitaxel and Compound A.

In some embodiments, the checkpoint inhibitor is administered at an amount of about 100 mg to about 500 mg in combination with paclitaxel and Compound A.

In some embodiments, the checkpoint inhibitor is administered at about 100 mg to about 450 mg in combination with paclitaxel and Compound A.

In some embodiments, the checkpoint inhibitor is administered at about 100 mg to about 400 mg in combination with paclitaxel and Compound A.

In some embodiments, the checkpoint inhibitor is administered at about 100 mg to about 350 mg in combination with paclitaxel and Compound A.

In some embodiments, the checkpoint inhibitor is administered at about 100 mg to about 300 mg in combination with paclitaxel and Compound A.

In some embodiments, the checkpoint inhibitor is administered at about 100 mg to about 290 mg in combination with paclitaxel and Compound A.

In some embodiments, the checkpoint inhibitor is administered at about 100 mg to about 280 mg in combination with paclitaxel and Compound A.

In some embodiments, the checkpoint inhibitor is administered at about 100 mg to about 270 mg in combination with paclitaxel and Compound A.

In some embodiments, the checkpoint inhibitor is administered at about 100 mg to about 260 mg in combination with paclitaxel and Compound A.

In some embodiments, the checkpoint inhibitor is administered at about 100 mg to about 250 mg in combination with paclitaxel and Compound A.

In some embodiments, the checkpoint inhibitor is administered at about 100 mg to about 240 mg in combination with paclitaxel and Compound A.

In some embodiments, the checkpoint inhibitor is administered at about 100 mg to about 230 mg in combination with paclitaxel and Compound A.

In some embodiments, the checkpoint inhibitor is administered at about 100 mg to about 220 mg in combination with paclitaxel and Compound A.

In some embodiments, the checkpoint inhibitor is administered at about 100 mg to about 210 mg in combination with paclitaxel and Compound A.

In some embodiments, the checkpoint inhibitor is administered at about 100 mg to about 200 mg in combination with paclitaxel and Compound A.

In some embodiments, the checkpoint inhibitor is administered at about 100 mg to about 190 mg in combination with paclitaxel and Compound A.

In some embodiments, the checkpoint inhibitor is administered at about 100 mg to about 180 mg in combination with paclitaxel and Compound A.

In some embodiments, the checkpoint inhibitor is administered at about 100 mg to about 170 mg in combination with paclitaxel and Compound A.

In some embodiments, the checkpoint inhibitor is administered at about 100 mg to about 160 mg in combination with paclitaxel and Compound A.

In some embodiments, the checkpoint inhibitor is administered at about 100 mg to about 150 mg in combination with paclitaxel and Compound A.

In some embodiments, the checkpoint inhibitor is administered at about 100 mg to about 140 mg in combination with paclitaxel and Compound A.

In some embodiments, the checkpoint inhibitor is administered at about 100 mg to about 130 mg in combination with paclitaxel and Compound A.

In some embodiments, the checkpoint inhibitor is administered at about 100 mg to about 120 mg in combination with paclitaxel and Compound A.

In some embodiments, the checkpoint inhibitor is administered at about 100 mg to about 110 mg in combination with paclitaxel and Compound A.

In some embodiments, the checkpoint inhibitor is administered at an amount of about 150 mg to about 500 mg in combination with paclitaxel and Compound A.

In some embodiments, the checkpoint inhibitor is administered at about 150 mg to about 450 mg in combination with paclitaxel and Compound A.

In some embodiments, the checkpoint inhibitor is administered at about 150 mg to about 400 mg in combination with paclitaxel and Compound A.

In some embodiments, the checkpoint inhibitor is administered at about 150 mg to about 350 mg in combination with paclitaxel and Compound A.

In some embodiments, the checkpoint inhibitor is administered at about 150 mg to about 300 mg in combination with paclitaxel and Compound A.

In some embodiments, the checkpoint inhibitor is administered at about 150 mg to about 290 mg in combination with paclitaxel and Compound A.

In some embodiments, the checkpoint inhibitor is administered at about 150 mg to about 280 mg in combination with paclitaxel and Compound A.

In some embodiments, the checkpoint inhibitor is administered at about 150 mg to about 270 mg in combination with paclitaxel and Compound A.

In some embodiments, the checkpoint inhibitor is administered at about 150 mg to about 260 mg in combination with paclitaxel and Compound A.

In some embodiments, the checkpoint inhibitor is administered at about 150 mg to about 250 mg in combination with paclitaxel and Compound A.

In some embodiments, the checkpoint inhibitor is administered at about 150 mg to about 240 mg in combination with paclitaxel and Compound A.

In some embodiments, the checkpoint inhibitor is administered at about 150 mg to about 230 mg in combination with paclitaxel and Compound A.

In some embodiments, the checkpoint inhibitor is administered at about 150 mg to about 220 mg in combination with paclitaxel and Compound A.

In some embodiments, the checkpoint inhibitor is administered at about 150 mg to about 210 mg in combination with paclitaxel and Compound A.

In some embodiments, the checkpoint inhibitor is administered at about 150 mg to about 200 mg in combination with paclitaxel and Compound A.

In some embodiments, the checkpoint inhibitor is administered at about 150 mg to about 190 mg in combination with paclitaxel and Compound A.

In some embodiments, the checkpoint inhibitor is administered at about 150 mg to about 180 mg in combination with paclitaxel and Compound A.

In some embodiments, the checkpoint inhibitor is administered at about 150 mg to about 170 mg in combination with paclitaxel and Compound A.

In some embodiments, the checkpoint inhibitor is administered at about 150 mg to about 160 mg in combination with paclitaxel and Compound A.

In some embodiments, the checkpoint inhibitor is administered at an amount of about 200 mg to about 500 mg in combination with paclitaxel and Compound A.

In some embodiments, the checkpoint inhibitor is administered at about 200 mg to about 450 mg in combination with paclitaxel and Compound A.

In some embodiments, the checkpoint inhibitor is administered at about 200 mg to about 400 mg in combination with paclitaxel and Compound A.

In some embodiments, the checkpoint inhibitor is administered at about 200 mg to about 350 mg in combination with paclitaxel and Compound A.

In some embodiments, the checkpoint inhibitor is administered at about 200 mg to about 300 mg in combination with paclitaxel and Compound A.

In some embodiments, the checkpoint inhibitor is administered at about 200 mg to about 290 mg in combination with paclitaxel and Compound A.

In some embodiments, the checkpoint inhibitor is administered at about 200 mg to about 280 mg in combination with paclitaxel and Compound A.

In some embodiments, the checkpoint inhibitor is administered at about 200 mg to about 270 mg in combination with paclitaxel and Compound A.

In some embodiments, the checkpoint inhibitor is administered at about 200 mg to about 260 mg in combination with paclitaxel and Compound A.

In some embodiments, the checkpoint inhibitor is administered at about 200 mg to about 250 mg in combination with paclitaxel and Compound A.

In some embodiments, the checkpoint inhibitor is administered at about 200 mg to about 240 mg in combination with paclitaxel and Compound A.

In some embodiments, the checkpoint inhibitor is administered at about 200 mg to about 230 mg in combination with paclitaxel and Compound A.

In some embodiments, the checkpoint inhibitor is administered at about 200 mg to about 220 mg in combination with paclitaxel and Compound A.

In some embodiments, the checkpoint inhibitor is administered at about 200 mg to about 210 mg in combination with paclitaxel and Compound A.

In some embodiments, the checkpoint inhibitor is administered at about 500 mg in combination with paclitaxel and Compound A.

In some embodiments, the checkpoint inhibitor is administered at about 450 mg in combination with paclitaxel and Compound A.

In some embodiments, the checkpoint inhibitor is administered at about 440 mg in combination with paclitaxel and Compound A.

In some embodiments, the checkpoint inhibitor is administered at about 430 mg in combination with paclitaxel and Compound A.

In some embodiments, the checkpoint inhibitor is administered at about 420 mg in combination with paclitaxel and Compound A.

In some embodiments, the checkpoint inhibitor is administered at about 410 mg in combination with paclitaxel and Compound A.

In some embodiments, the checkpoint inhibitor is administered at about 400 mg in combination with paclitaxel and Compound A.

In some embodiments, the checkpoint inhibitor is administered at about 390 mg in combination with paclitaxel and Compound A.

In some embodiments, the checkpoint inhibitor is administered at about 380 mg in combination with paclitaxel and Compound A.

In some embodiments, the checkpoint inhibitor is administered at about 370 mg in combination with paclitaxel and Compound A.

In some embodiments, the checkpoint inhibitor is administered at about 360 mg in combination with paclitaxel and Compound A.

In some embodiments, the checkpoint inhibitor is administered at about 350 mg in combination with paclitaxel and Compound A.

In some embodiments, the checkpoint inhibitor is administered at about 340 mg in combination with paclitaxel and Compound A.

In some embodiments, the checkpoint inhibitor is administered at about 330 mg in combination with paclitaxel and Compound A.

In some embodiments, the checkpoint inhibitor is administered at about 320 mg in combination with paclitaxel and Compound A.

In some embodiments, the checkpoint inhibitor is administered at about 310 mg in combination with paclitaxel and Compound A.

In some embodiments, the checkpoint inhibitor is administered at about 300 mg in combination with paclitaxel and Compound A.

In some embodiments, the checkpoint inhibitor is administered at about 290 mg in combination with paclitaxel and Compound A.

In some embodiments, the checkpoint inhibitor is administered at about 280 mg in combination with paclitaxel and Compound A.

In some embodiments, the checkpoint inhibitor is administered at about 270 mg in combination with paclitaxel and Compound A.

In some embodiments, the checkpoint inhibitor is administered at about 260 mg in combination with paclitaxel and Compound A.

In some embodiments, the checkpoint inhibitor is administered at about 250 mg in combination with paclitaxel and Compound A.

In some embodiments, the checkpoint inhibitor is administered at about 240 mg in combination with paclitaxel and Compound A.

In some embodiments, the checkpoint inhibitor is administered at about 230 mg in combination with paclitaxel and Compound A.

In some embodiments, the checkpoint inhibitor is administered at about 220 mg in combination with paclitaxel and Compound A.

In some embodiments, the checkpoint inhibitor is administered at about 210 mg in combination with paclitaxel and Compound A.

In some embodiments, the checkpoint inhibitor is administered at about 200 mg in combination with paclitaxel and Compound A.

In some embodiments, the checkpoint inhibitor is administered at about 190 mg in combination with paclitaxel and Compound A.

In some embodiments, the checkpoint inhibitor is administered at about 180 mg in combination with paclitaxel and Compound A.

In some embodiments, the checkpoint inhibitor is administered at about 170 mg in combination with paclitaxel and Compound A.

In some embodiments, the checkpoint inhibitor is administered at about 160 mg in combination with paclitaxel and Compound A.

In some embodiments, the checkpoint inhibitor is administered at about 150 mg in combination with paclitaxel and Compound A.

In some embodiments, the checkpoint inhibitor is administered at about 140 mg in combination with paclitaxel and Compound A.

In some embodiments, the checkpoint inhibitor is administered at about 130 mg in combination with paclitaxel and Compound A.

In some embodiments, the checkpoint inhibitor is administered at about 120 mg in combination with paclitaxel and Compound A.

In some embodiments, the checkpoint inhibitor is administered at about 110 mg in combination with paclitaxel and Compound A.

In some embodiments, the checkpoint inhibitor is administered at about 100 mg in combination with paclitaxel and Compound A.

In some embodiments, the checkpoint inhibitor is administered at about 50 mg in combination with paclitaxel and Compound A.

In some embodiments, the checkpoint inhibitor is administered on consecutive days in combination with paclitaxel and Compound A.

In some embodiments, the checkpoint inhibitor is administered 1-7 times per week in combination with paclitaxel and Compound A.

In some embodiments, the checkpoint inhibitor is administered 2-7 times per week in combination with paclitaxel and Compound A.

In some embodiments, the checkpoint inhibitor is administered 3-7 times per week in combination with paclitaxel and Compound A.

In some embodiments, the checkpoint inhibitor is administered 4-7 times per week in combination with paclitaxel and Compound A.

In some embodiments, the checkpoint inhibitor is administered 5-7 times per week in combination with paclitaxel and Compound A.

In some embodiments, the checkpoint inhibitor is administered 6-7 times per week in combination with paclitaxel and Compound A.

In some embodiments, the checkpoint inhibitor is administered 1-6 times per week in combination with paclitaxel and Compound A. In some embodiments, the checkpoint inhibitor is administered 1-5 times per week in combination with paclitaxel and Compound A. In some embodiments, the checkpoint inhibitor is administered 1-4 times per week in combination with paclitaxel and Compound A. In some embodiments, the checkpoint inhibitor is administered 1-3 times per week in combination with paclitaxel and Compound A. In some embodiments, the checkpoint inhibitor is administered 1-2 times per week in combination with paclitaxel and Compound A.

In some embodiments, the checkpoint inhibitor is administered 2-6 times per week in combination with paclitaxel and Compound A. In some embodiments, the checkpoint inhibitor is administered 2-5 times per week in combination with paclitaxel and Compound A. In some embodiments, the checkpoint inhibitor is administered 2-4 times per week in combination with paclitaxel and Compound A. In some embodiments, the checkpoint inhibitor is administered 2-3 times per week in combination with paclitaxel and Compound A.

In some embodiments, the checkpoint inhibitor is administered 3-6 times per week in combination with paclitaxel and Compound A. In some embodiments, the checkpoint inhibitor is administered 3-5 times per week in combination with paclitaxel and Compound A. In some embodiments, the checkpoint inhibitor is administered 3-4 times per week in combination with paclitaxel and Compound A.

In some embodiments, the checkpoint inhibitor is administered 4-6 times per week in combination with paclitaxel and Compound A. In some embodiments, the checkpoint inhibitor is administered 4-5 times per week in combination with paclitaxel and Compound A.

In some embodiments, the checkpoint inhibitor is administered 5-6 times per week in combination with paclitaxel and Compound A.

In some embodiments, the checkpoint inhibitor is administered at least 1 time per week in combination with paclitaxel and Compound A.

In some embodiments, the checkpoint inhibitor is administered at least 2 times per week in combination with paclitaxel and Compound A.

In some embodiments, the checkpoint inhibitor is administered at least 3 times per week in combination with paclitaxel and Compound A.

In some embodiments, the checkpoint inhibitor is administered at least 4 times per week in combination with paclitaxel and Compound A.

In some embodiments, the checkpoint inhibitor is administered at least 5 times per week in combination with paclitaxel and Compound A.

In some embodiments, the checkpoint inhibitor is administered at least 6 times per week in combination with paclitaxel and Compound A.

In some embodiments, the checkpoint inhibitor is administered daily per week in combination with paclitaxel and Compound A.

In some embodiments, the checkpoint inhibitor is administered at least 1 time per week at an amount of about 1 mg to about 500 mg in combination with paclitaxel and Compound A.

In some embodiments, the checkpoint inhibitor is administered at least 1 time per week at an amount of about 1 mg to about 400 mg in combination with paclitaxel and Compound A.

In some embodiments, the checkpoint inhibitor is administered at least 1 time per week at an amount of about 1 mg to about 300 mg in combination with paclitaxel and Compound A.

In some embodiments, the checkpoint inhibitor is administered at least 1 time per week at an amount of about 1 mg to about 200 mg in combination with paclitaxel and Compound A.

In some embodiments, the checkpoint inhibitor is administered at least 1 time per week at an amount of about 10 mg to about 500 mg in combination with paclitaxel and Compound A.

In some embodiments, the checkpoint inhibitor is administered at least 1 time per week at an amount of about 200 mg to about 500 mg in combination with paclitaxel and Compound A.

In some embodiments, the checkpoint inhibitor is administered at least 1 time per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 1 time per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 1 time per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 1 time per week at an amount of about 400 mg in combination with paclitaxel and Compound A. In some embodiments, the checkpoint inhibitor is administered at least 1 time per week at an amount of about 200 mg in combination with paclitaxel and Compound A.

In some embodiments, the checkpoint inhibitor is administered at least 2 times per week at an amount of about 1 mg to about 500 mg in combination with paclitaxel and Compound A.

In some embodiments, the checkpoint inhibitor is administered at least 2 times per week at an amount of about 1 mg to about 400 mg in combination with paclitaxel and Compound A.

In some embodiments, the checkpoint inhibitor is administered at least 2 times per week at an amount of about 1 mg to about 300 mg in combination with paclitaxel and Compound A.

In some embodiments, the checkpoint inhibitor is administered at least 2 times per week at an amount of about 1 mg to about 200 mg in combination with paclitaxel and Compound A.

In some embodiments, the checkpoint inhibitor is administered at least 2 times per week at an amount of about 10 mg to about 500 mg in combination with paclitaxel and Compound A.

In some embodiments, the checkpoint inhibitor is administered at least 2 times per week at an amount of about 200 mg to about 500 mg in combination with paclitaxel and Compound A.

In some embodiments, the checkpoint inhibitor is administered at least 2 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 2 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 2 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 2 times per week at an amount of about 400 mg in combination with paclitaxel and Compound A. In some embodiments, the checkpoint inhibitor is administered at least 2 times per week at an amount of about 200 mg in combination with paclitaxel and Compound A.

In some embodiments, the checkpoint inhibitor is administered at least 3 times per week at an amount of about 1 mg to about 500 mg in combination with paclitaxel and Compound A.

In some embodiments, the checkpoint inhibitor is administered at least 3 times per week at an amount of about 1 mg to about 400 mg in combination with paclitaxel and Compound A.

In some embodiments, the checkpoint inhibitor is administered at least 3 times per week at an amount of about 1 mg to about 300 mg in combination with paclitaxel and Compound A.

In some embodiments, the checkpoint inhibitor is administered at least 3 times per week at an amount of about 1 mg to about 200 mg in combination with paclitaxel and Compound A.

In some embodiments, the checkpoint inhibitor is administered at least 3 times per week at an amount of about 10 mg to about 500 mg in combination with paclitaxel and Compound A.

In some embodiments, the checkpoint inhibitor is administered at least 3 times per week at an amount of about 200 mg to about 500 mg in combination with paclitaxel and Compound A.

In some embodiments, the checkpoint inhibitor is administered at least 3 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 3 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 3 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 3 times per week at an amount of about 400 mg in combination with paclitaxel and Compound A. In some embodiments, the checkpoint inhibitor is administered at least 3 times per week at an amount of about 200 mg in combination with paclitaxel and Compound A.

In some embodiments, the checkpoint inhibitor is administered at least 4 times per week at an amount of about 1 mg to about 500 mg in combination with paclitaxel and Compound A.

In some embodiments, the checkpoint inhibitor is administered at least 4 times per week at an amount of about 1 mg to about 400 mg in combination with paclitaxel and Compound A.

In some embodiments, the checkpoint inhibitor is administered at least 4 times per week at an amount of about 1 mg to about 300 mg in combination with paclitaxel and Compound A.

In some embodiments, the checkpoint inhibitor is administered at least 4 times per week at an amount of about 1 mg to about 200 mg in combination with paclitaxel and Compound A.

In some embodiments, the checkpoint inhibitor is administered at least 4 times per week at an amount of about 10 mg to about 500 mg in combination with paclitaxel and Compound A.

In some embodiments, the checkpoint inhibitor is administered at least 4 times per week at an amount of about 200 mg to about 500 mg in combination with paclitaxel and Compound A.

In some embodiments, the checkpoint inhibitor is administered at least 4 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 4 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 4 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 4 times per week at an amount of about 400 mg in combination with paclitaxel and Compound A. In some embodiments, the checkpoint inhibitor is administered at least 4 times per week at an amount of about 200 mg in combination with paclitaxel and Compound A.

In some embodiments, the checkpoint inhibitor is administered at least 5 times per week at an amount of about 1 mg to about 500 mg in combination with paclitaxel and Compound A.

In some embodiments, the checkpoint inhibitor is administered at least 5 times per week at an amount of about 1 mg to about 400 mg in combination with paclitaxel and Compound A.

In some embodiments, the checkpoint inhibitor is administered at least 5 times per week at an amount of about 1 mg to about 300 mg in combination with paclitaxel and Compound A.

In some embodiments, the checkpoint inhibitor is administered at least 5 times per week at an amount of about 1 mg to about 200 mg in combination with paclitaxel and Compound A.

In some embodiments, the checkpoint inhibitor is administered at least 5 times per week at an amount of about 10 mg to about 500 mg in combination with paclitaxel and Compound A.

In some embodiments, the checkpoint inhibitor is administered at least 5 times per week at an amount of about 200 mg to about 500 mg in combination with paclitaxel and Compound A.

In some embodiments, the checkpoint inhibitor is administered at least 5 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 5 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 5 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 5 times per week at an amount of about 400 mg in combination with paclitaxel and Compound A. In some embodiments, the checkpoint inhibitor is administered at least 5 times per week at an amount of about 200 mg in combination with paclitaxel and Compound A.

In some embodiments, the checkpoint inhibitor is administered at least 6 times per week at an amount of about 1 mg to about 500 mg in combination with paclitaxel and Compound A.

In some embodiments, the checkpoint inhibitor is administered at least 6 times per week at an amount of about 1 mg to about 400 mg in combination with paclitaxel and Compound A.

In some embodiments, the checkpoint inhibitor is administered at least 6 times per week at an amount of about 1 mg to about 300 mg in combination with paclitaxel and Compound A.

In some embodiments, the checkpoint inhibitor is administered at least 6 times per week at an amount of about 1 mg to about 200 mg in combination with paclitaxel and Compound A.

In some embodiments, the checkpoint inhibitor is administered at least 6 times per week at an amount of about 10 mg to about 500 mg in combination with paclitaxel and Compound A.

In some embodiments, the checkpoint inhibitor is administered at least 6 times per week at an amount of about 200 mg to about 500 mg in combination with paclitaxel and Compound A.

In some embodiments, the checkpoint inhibitor is administered at least 6 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, the checkpoint inhibitor is administered at least 6 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, the checkpoint inhibitor is administered at least 6 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, the checkpoint inhibitor is administered at least 6 times per week at an amount of about 400 mg in combination with paclitaxel and Compound A. In some embodiments, the checkpoint inhibitor is administered at least 6 times per week at an amount of about 200 mg in combination with paclitaxel and Compound A.

In some embodiments, the checkpoint inhibitor is administered once every three weeks in combination with paclitaxel and Compound A.

In some embodiments, the checkpoint inhibitor is administered once every six weeks in combination with paclitaxel and Compound A.

In some embodiments, the checkpoint inhibitor is administered once every three weeks from about 1 mg to about 500 mg in combination with paclitaxel and Compound A.

In some embodiments, the checkpoint inhibitor is administered once every six weeks about 1 mg to about 500 mg in combination with paclitaxel and Compound A.

In some embodiments, the checkpoint inhibitor is administered once every three weeks from about 1 mg to about 400 mg in combination with paclitaxel and Compound A.

In some embodiments, the checkpoint inhibitor is administered once every six weeks from about 1 mg to about 400 mg in combination with paclitaxel and Compound A.

In some embodiments, the checkpoint inhibitor is administered once every three weeks from about 1 mg to about 300 mg in combination with paclitaxel and Compound A.

In some embodiments, the checkpoint inhibitor is administered once every six weeks from about 1 mg to about 300 mg in combination with paclitaxel and Compound A.

In some embodiments, the checkpoint inhibitor is administered once every three weeks from about 1 mg to about 200 mg in combination with paclitaxel and Compound A.

In some embodiments, the checkpoint inhibitor is administered once every six weeks from about 1 mg to about 200 mg in combination with paclitaxel and Compound A.

In some embodiments, the checkpoint inhibitor is administered once every three weeks from about 10 mg to about 500 mg in combination with paclitaxel and Compound A.

In some embodiments, the checkpoint inhibitor is administered once every six weeks from about 10 mg to about 500 mg in combination with paclitaxel and Compound A.

In some embodiments, the checkpoint inhibitor is administered once every three weeks from about 200 mg to about 500 mg in combination with paclitaxel and Compound A.

In some embodiments, the checkpoint inhibitor is administered once every six weeks from about 200 mg to about 500 mg in combination with paclitaxel and Compound A.

In some embodiments, the checkpoint inhibitor is administered once every three weeks at about 200 mg in combination with paclitaxel and Compound A.

In some embodiments, the checkpoint inhibitor is administered once every six weeks at about 200 mg in combination with paclitaxel and Compound A.

In some embodiments, the checkpoint inhibitor is administered once every three weeks at about 400 mg in combination with paclitaxel and Compound A.

In some embodiments, the checkpoint inhibitor is administered once every six weeks at about 400 mg in combination with paclitaxel and Compound A.

In some embodiments, pembrolizumab is administered at an amount of about 1 mg to about 500 mg in combination with paclitaxel and Compound A.

In some embodiments, pembrolizumab is administered at about 1 mg to about 400 mg in combination with paclitaxel and Compound A.

In some embodiments, pembrolizumab is administered at about 1 mg to about 300 mg in combination with paclitaxel and Compound A.

In some embodiments, pembrolizumab is administered at about 1 mg to about 200 mg in combination with paclitaxel and Compound A.

In some embodiments, pembrolizumab is administered at an amount of about 100 mg to about 500 mg in combination with paclitaxel and Compound A.

In some embodiments, pembrolizumab is administered at about 100 mg to about 450 mg in combination with paclitaxel and Compound A.

In some embodiments, pembrolizumab is administered at about 100 mg to about 400 mg in combination with paclitaxel and Compound A.

In some embodiments, pembrolizumab is administered at about 100 mg to about 350 mg in combination with paclitaxel and Compound A.

In some embodiments, pembrolizumab is administered at about 100 mg to about 300 mg in combination with paclitaxel and Compound A.

In some embodiments, pembrolizumab is administered at about 100 mg to about 290 mg in combination with paclitaxel and Compound A.

In some embodiments, pembrolizumab is administered at about 100 mg to about 280 mg in combination with paclitaxel and Compound A.

In some embodiments, pembrolizumab is administered at about 100 mg to about 270 mg in combination with paclitaxel and Compound A.

In some embodiments, pembrolizumab is administered at about 100 mg to about 260 mg in combination with paclitaxel and Compound A.

In some embodiments, pembrolizumab is administered at about 100 mg to about 250 mg in combination with paclitaxel and Compound A.

In some embodiments, pembrolizumab is administered at about 100 mg to about 240 mg in combination with paclitaxel and Compound A.

In some embodiments, pembrolizumab is administered at about 100 mg to about 230 mg in combination with paclitaxel and Compound A.

In some embodiments, pembrolizumab is administered at about 100 mg to about 220 mg in combination with paclitaxel and Compound A.

In some embodiments, pembrolizumab is administered at about 100 mg to about 210 mg in combination with paclitaxel and Compound A.

In some embodiments, pembrolizumab is administered at about 100 mg to about 200 mg in combination with paclitaxel and Compound A.

In some embodiments, pembrolizumab is administered at about 100 mg to about 190 mg in combination with paclitaxel and Compound A.

In some embodiments, pembrolizumab is administered at about 100 mg to about 180 mg in combination with paclitaxel and Compound A.

In some embodiments, pembrolizumab is administered at about 100 mg to about 170 mg in combination with paclitaxel and Compound A.

In some embodiments, pembrolizumab is administered at about 100 mg to about 160 mg in combination with paclitaxel and Compound A.

In some embodiments, pembrolizumab is administered at about 100 mg to about 150 mg in combination with paclitaxel and Compound A.

In some embodiments, pembrolizumab is administered at about 100 mg to about 140 mg in combination with paclitaxel and Compound A.

In some embodiments, pembrolizumab is administered at about 100 mg to about 130 mg in combination with paclitaxel and Compound A.

In some embodiments, pembrolizumab is administered at about 100 mg to about 120 mg in combination with paclitaxel and Compound A.

In some embodiments, pembrolizumab is administered at about 100 mg to about 110 mg in combination with paclitaxel and Compound A.

In some embodiments, pembrolizumab is administered at an amount of about 150 mg to about 500 mg in combination with paclitaxel and Compound A.

In some embodiments, pembrolizumab is administered at about 150 mg to about 450 mg in combination with paclitaxel and Compound A.

In some embodiments, pembrolizumab is administered at about 150 mg to about 400 mg in combination with paclitaxel and Compound A.

In some embodiments, pembrolizumab is administered at about 150 mg to about 350 mg in combination with paclitaxel and Compound A.

In some embodiments, pembrolizumab is administered at about 150 mg to about 300 mg in combination with paclitaxel and Compound A.

In some embodiments, pembrolizumab is administered at about 150 mg to about 290 mg in combination with paclitaxel and Compound A.

In some embodiments, pembrolizumab is administered at about 150 mg to about 280 mg in combination with paclitaxel and Compound A.

In some embodiments, pembrolizumab is administered at about 150 mg to about 270 mg in combination with paclitaxel and Compound A.

In some embodiments, pembrolizumab is administered at about 150 mg to about 260 mg in combination with paclitaxel and Compound A.

In some embodiments, pembrolizumab is administered at about 150 mg to about 250 mg in combination with paclitaxel and Compound A.

In some embodiments, pembrolizumab is administered at about 150 mg to about 240 mg in combination with paclitaxel and Compound A.

In some embodiments, pembrolizumab is administered at about 150 mg to about 230 mg in combination with paclitaxel and Compound A.

In some embodiments, pembrolizumab is administered at about 150 mg to about 220 mg in combination with paclitaxel and Compound A.

In some embodiments, pembrolizumab is administered at about 150 mg to about 210 mg in combination with paclitaxel and Compound A.

In some embodiments, pembrolizumab is administered at about 150 mg to about 200 mg in combination with paclitaxel and Compound A.

In some embodiments, pembrolizumab is administered at about 150 mg to about 190 mg in combination with paclitaxel and Compound A.

In some embodiments, pembrolizumab is administered at about 150 mg to about 180 mg in combination with paclitaxel and Compound A.

In some embodiments, pembrolizumab is administered at about 150 mg to about 170 mg in combination with paclitaxel and Compound A.

In some embodiments, pembrolizumab is administered at about 150 mg to about 160 mg in combination with paclitaxel and Compound A.

In some embodiments, pembrolizumab is administered at an amount of about 200 mg to about 500 mg in combination with paclitaxel and Compound A.

In some embodiments, pembrolizumab is administered at about 200 mg to about 450 mg in combination with paclitaxel and Compound A.

In some embodiments, pembrolizumab is administered at about 200 mg to about 400 mg in combination with paclitaxel and Compound A.

In some embodiments, pembrolizumab is administered at about 200 mg to about 350 mg in combination with paclitaxel and Compound A.

In some embodiments, pembrolizumab is administered at about 200 mg to about 300 mg in combination with paclitaxel and Compound A.

In some embodiments, pembrolizumab is administered at about 200 mg to about 290 mg in combination with paclitaxel and Compound A.

In some embodiments, pembrolizumab is administered at about 200 mg to about 280 mg in combination with paclitaxel and Compound A.

In some embodiments, pembrolizumab is administered at about 200 mg to about 270 mg in combination with paclitaxel and Compound A.

In some embodiments, pembrolizumab is administered at about 200 mg to about 260 mg in combination with paclitaxel and Compound A.

In some embodiments, pembrolizumab is administered at about 200 mg to about 250 mg in combination with paclitaxel and Compound A.

In some embodiments, pembrolizumab is administered at about 200 mg to about 240 mg in combination with paclitaxel and Compound A.

In some embodiments, pembrolizumab is administered at about 200 mg to about 230 mg in combination with paclitaxel and Compound A.

In some embodiments, pembrolizumab is administered at about 200 mg to about 220 mg in combination with paclitaxel and Compound A.

In some embodiments, pembrolizumab is administered at about 200 mg to about 210 mg in combination with paclitaxel and Compound A.

In some embodiments, pembrolizumab is administered at about 500 mg in combination with paclitaxel and Compound A.

In some embodiments, pembrolizumab is administered at about 450 mg in combination with paclitaxel and Compound A.

In some embodiments, pembrolizumab is administered at about 440 mg in combination with paclitaxel and Compound A.

In some embodiments, pembrolizumab is administered at about 430 mg in combination with paclitaxel and Compound A.

In some embodiments, pembrolizumab is administered at about 420 mg in combination with paclitaxel and Compound A.

In some embodiments, pembrolizumab is administered at about 410 mg in combination with paclitaxel and Compound A.

In some embodiments, pembrolizumab is administered at about 400 mg in combination with paclitaxel and Compound A.

In some embodiments, pembrolizumab is administered at about 390 mg in combination with paclitaxel and Compound A.

In some embodiments, pembrolizumab is administered at about 380 mg in combination with paclitaxel and Compound A.

In some embodiments, pembrolizumab is administered at about 370 mg in combination with paclitaxel and Compound A.

In some embodiments, pembrolizumab is administered at about 360 mg in combination with paclitaxel and Compound A.

In some embodiments, pembrolizumab is administered at about 350 mg in combination with paclitaxel and Compound A.

In some embodiments, pembrolizumab is administered at about 340 mg in combination with paclitaxel and Compound A.

In some embodiments, pembrolizumab is administered at about 330 mg in combination with paclitaxel and Compound A.

In some embodiments, pembrolizumab is administered at about 320 mg In some embodiments, pembrolizumab is administered at about 310 mg in combination with paclitaxel and Compound A.

In some embodiments, pembrolizumab is administered at about 300 mg in combination with paclitaxel and Compound A.

In some embodiments, pembrolizumab is administered at about 290 mg in combination with paclitaxel and Compound A.

In some embodiments, pembrolizumab is administered at about 280 mg in combination with paclitaxel and Compound A.

In some embodiments, pembrolizumab is administered at about 270 mg in combination with paclitaxel and Compound A.

In some embodiments, pembrolizumab is administered at about 260 mg in combination with paclitaxel and Compound A.

In some embodiments, pembrolizumab is administered at about 250 mg in combination with paclitaxel and Compound A.

In some embodiments, pembrolizumab is administered at about 240 mg in combination with paclitaxel and Compound A.

In some embodiments, pembrolizumab is administered at about 230 mg in combination with paclitaxel and Compound A.

In some embodiments, pembrolizumab is administered at about 220 mg in combination with paclitaxel and Compound A.

In some embodiments, pembrolizumab is administered at about 210 mg in combination with paclitaxel and Compound A.

In some embodiments, pembrolizumab is administered at about 200 mg in combination with paclitaxel and Compound A.

In some embodiments, pembrolizumab is administered at about 190 mg in combination with paclitaxel and Compound A.

In some embodiments, pembrolizumab is administered at about 180 mg in combination with paclitaxel and Compound A.

In some embodiments, pembrolizumab is administered at about 170 mg in combination with paclitaxel and Compound A.

In some embodiments, pembrolizumab is administered at about 160 mg in combination with paclitaxel and Compound A.

In some embodiments, pembrolizumab is administered at about 150 mg in combination with paclitaxel and Compound A.

In some embodiments, pembrolizumab is administered at about 140 mg in combination with paclitaxel and Compound A.

In some embodiments, pembrolizumab is administered at about 130 mg in combination with paclitaxel and Compound A.

In some embodiments, pembrolizumab is administered at about 120 mg In some embodiments, pembrolizumab is administered at about 110 mg in combination with paclitaxel and Compound A.

In some embodiments, pembrolizumab is administered at about 100 mg in combination with paclitaxel and Compound A.

In some embodiments, pembrolizumab is administered at about 50 mg in combination with paclitaxel and Compound A.

In some embodiments, pembrolizumab in the present application is formulated for intravenous administration at the various doses described herein with the various dosing frequencies in the methods, compositions, medicaments, therapeutic combinations, combinational therapies, and uses, described herein in combination with paclitaxel and Compound A.

In some embodiments, pembrolizumab is administered on consecutive days in combination with paclitaxel and Compound A.

In some embodiments, pembrolizumab is administered 1-7 times per week in combination with paclitaxel and Compound A.

In some embodiments, pembrolizumab is administered 2-7 times per week in combination with paclitaxel and Compound A.

In some embodiments, pembrolizumab is administered 3-7 times per week in combination with paclitaxel and Compound A.

In some embodiments, pembrolizumab is administered 4-7 times per week in combination with paclitaxel and Compound A.

In some embodiments, pembrolizumab is administered 5-7 times per week in combination with paclitaxel and Compound A.

In some embodiments, pembrolizumab is administered 6-7 times per week in combination with paclitaxel and Compound A.

In some embodiments, pembrolizumab is administered 1-6 times per week in combination with paclitaxel and Compound A. In some embodiments, pembrolizumab is administered 1-5 times per week in combination with paclitaxel and Compound A. In some embodiments, pembrolizumab is administered 1-4 times per week in combination with paclitaxel and Compound A. In some embodiments, pembrolizumab is administered 1-3 times per week in combination with paclitaxel and Compound A. In some embodiments, pembrolizumab is administered 1-2 times per week in combination with paclitaxel and Compound A.

In some embodiments, pembrolizumab is administered 2-6 times per week in combination with paclitaxel and Compound A. In some embodiments, pembrolizumab is administered 2-5 times per week in combination with paclitaxel and Compound A. In some embodiments, pembrolizumab is administered 2-4 times per week in combination with paclitaxel and Compound A. In some embodiments, pembrolizumab is administered 2-3 times per week in combination with paclitaxel and Compound A.

In some embodiments, pembrolizumab is administered 3-6 times per week in combination with paclitaxel and Compound A. In some embodiments, pembrolizumab is administered 3-5 times per week in combination with paclitaxel and Compound A. In some embodiments, pembrolizumab is administered 3-4 times per week in combination with paclitaxel and Compound A.

In some embodiments, pembrolizumab is administered 4-6 times per week in combination with paclitaxel and Compound A. In some embodiments, pembrolizumab is administered 4-5 times per week in combination with paclitaxel and Compound A.

In some embodiments, pembrolizumab is administered 5-6 times per week in combination with paclitaxel and Compound A.

In some embodiments, pembrolizumab is administered at least 1 time per week in combination with paclitaxel and Compound A.

In some embodiments, pembrolizumab is administered at least 2 times per week in combination with paclitaxel and Compound A.

In some embodiments, pembrolizumab is administered at least 3 times per week in combination with paclitaxel and Compound A.

In some embodiments, pembrolizumab is administered at least 4 times per week in combination with paclitaxel and Compound A.

In some embodiments, pembrolizumab is administered at least 5 times per week in combination with paclitaxel and Compound A.

In some embodiments, pembrolizumab is administered at least 6 times per week in combination with paclitaxel and Compound A.

In some embodiments, pembrolizumab is administered daily per week in combination with paclitaxel and Compound A.

In some embodiments, pembrolizumab is administered at least 1 time per week at an amount of about 1 mg to about 500 mg in combination with paclitaxel and Compound A.

In some embodiments, pembrolizumab is administered at least 1 time per week at an amount of about 1 mg to about 400 mg in combination with paclitaxel and Compound A.

In some embodiments, pembrolizumab is administered at least 1 time per week at an amount of about 1 mg to about 300 mg in combination with paclitaxel and Compound A.

In some embodiments, pembrolizumab is administered at least 1 time per week at an amount of about 1 mg to about 200 mg in combination with paclitaxel and Compound A.

In some embodiments, pembrolizumab is administered at least 1 time per week at an amount of about 10 mg to about 500 mg in combination with paclitaxel and Compound A.

In some embodiments, pembrolizumab is administered at least 1 time per week at an amount of about 200 mg to about 500 mg in combination with paclitaxel and Compound A.

In some embodiments, pembrolizumab is administered at least 1 time per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 1 time per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 1 time per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 1 time per week at an amount of about 400 mg in combination with paclitaxel and Compound A. In some embodiments, pembrolizumab is administered at least 1 time per week at an amount of about 200 mg in combination with paclitaxel and Compound A.

In some embodiments, pembrolizumab is administered at least 2 times per week at an amount of about 1 mg to about 500 mg in combination with paclitaxel and Compound A.

In some embodiments, pembrolizumab is administered at least 2 times per week at an amount of about 1 mg to about 400 mg in combination with paclitaxel and Compound A.

In some embodiments, pembrolizumab is administered at least 2 times per week at an amount of about 1 mg to about 300 mg in combination with paclitaxel and Compound A.

In some embodiments, pembrolizumab is administered at least 2 times per week at an amount of about 1 mg to about 200 mg in combination with paclitaxel and Compound A.

In some embodiments, pembrolizumab is administered at least 2 times per week at an amount of about 10 mg to about 500 mg in combination with paclitaxel and Compound A.

In some embodiments, pembrolizumab is administered at least 2 times per week at an amount of about 200 mg to about 500 mg in combination with paclitaxel and Compound A.

In some embodiments, pembrolizumab is administered at least 2 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 2 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 2 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 2 times per week at an amount of about 400 mg in combination with paclitaxel and Compound A. In some embodiments, pembrolizumab is administered at least 2 times per week at an amount of about 200 mg in combination with paclitaxel and Compound A.

In some embodiments, pembrolizumab is administered at least 3 times per week at an amount of about 1 mg to about 500 mg in combination with paclitaxel and Compound A.

In some embodiments, pembrolizumab is administered at least 3 times per week at an amount of about 1 mg to about 400 mg in combination with paclitaxel and Compound A.

In some embodiments, pembrolizumab is administered at least 3 times per week at an amount of about 1 mg to about 300 mg in combination with paclitaxel and Compound A.

In some embodiments, pembrolizumab is administered at least 3 times per week at an amount of about 1 mg to about 200 mg in combination with paclitaxel and Compound A.

In some embodiments, pembrolizumab is administered at least 3 times per week at an amount of about 10 mg to about 500 mg in combination with paclitaxel and Compound A.

In some embodiments, pembrolizumab is administered at least 3 times per week at an amount of about 200 mg to about 500 mg in combination with paclitaxel and Compound A.

In some embodiments, pembrolizumab is administered at least 3 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 3 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 3 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 3 times per week at an amount of about 400 mg in combination with paclitaxel and Compound A. In some embodiments, pembrolizumab is administered at least 3 times per week at an amount of about 200 mg in combination with paclitaxel and Compound A.

In some embodiments, pembrolizumab is administered at least 4 times per week at an amount of about 1 mg to about 500 mg in combination with paclitaxel and Compound A.

In some embodiments, pembrolizumab is administered at least 4 times per week at an amount of about 1 mg to about 400 mg in combination with paclitaxel and Compound A.

In some embodiments, pembrolizumab is administered at least 4 times per week at an amount of about 1 mg to about 300 mg in combination with paclitaxel and Compound A.

In some embodiments, pembrolizumab is administered at least 4 times per week at an amount of about 1 mg to about 200 mg in combination with paclitaxel and Compound A.

In some embodiments, pembrolizumab is administered at least 4 times per week at an amount of about 10 mg to about 500 mg in combination with paclitaxel and Compound A.

In some embodiments, pembrolizumab is administered at least 4 times per week at an amount of about 200 mg to about 500 mg in combination with paclitaxel and Compound A.

In some embodiments, pembrolizumab is administered at least 4 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 4 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 4 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 4 times per week at an amount of about 400 mg in combination with paclitaxel and Compound A. In some embodiments, pembrolizumab is administered at least 4 times per week at an amount of about 200 mg in combination with paclitaxel and Compound A.

In some embodiments, pembrolizumab is administered at least 5 times per week at an amount of about 1 mg to about 500 mg in combination with paclitaxel and Compound A.

In some embodiments, pembrolizumab is administered at least 5 times per week at an amount of about 1 mg to about 400 mg in combination with paclitaxel and Compound A.

In some embodiments, pembrolizumab is administered at least 5 times per week at an amount of about 1 mg to about 300 mg in combination with paclitaxel and Compound A.

In some embodiments, pembrolizumab is administered at least 5 times per week at an amount of about 1 mg to about 200 mg in combination with paclitaxel and Compound A.

In some embodiments, pembrolizumab is administered at least 5 times per week at an amount of about 10 mg to about 500 mg in combination with paclitaxel and Compound A.

In some embodiments, pembrolizumab is administered at least 5 times per week at an amount of about 200 mg to about 500 mg in combination with paclitaxel and Compound A.

In some embodiments, pembrolizumab is administered at least 5 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 5 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 5 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 5 times per week at an amount of about 400 mg in combination with paclitaxel and Compound A. In some embodiments, pembrolizumab is administered at least 5 times per week at an amount of about 200 mg in combination with paclitaxel and Compound A.

In some embodiments, pembrolizumab is administered at least 6 times per week at an amount of about 1 mg to about 500 mg in combination with paclitaxel and Compound A.

In some embodiments, pembrolizumab is administered at least 6 times per week at an amount of about 1 mg to about 400 mg in combination with paclitaxel and Compound A.

In some embodiments, pembrolizumab is administered at least 6 times per week at an amount of about 1 mg to about 300 mg in combination with paclitaxel and Compound A.

In some embodiments, pembrolizumab is administered at least 6 times per week at an amount of about 1 mg to about 200 mg in combination with paclitaxel and Compound A.

In some embodiments, pembrolizumab is administered at least 6 times per week at an amount of about 10 mg to about 500 mg in combination with paclitaxel and Compound A.

In some embodiments, pembrolizumab is administered at least 6 times per week at an amount of about 200 mg to about 500 mg in combination with paclitaxel and Compound A.

In some embodiments, pembrolizumab is administered at least 6 times per week at an amount of about 500 mg, about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, about 150 mg, about 140 mg, about 130 mg, about 120 mg, about 110 mg, about 100 mg, or about 50 mg in combination with paclitaxel and Compound A. In some embodiments, pembrolizumab is administered at least 6 times per week at an amount of about 450 mg, about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, about 200 mg, about 190 mg, about 180 mg, about 170 mg, about 160 mg, or about 150 mg in combination with paclitaxel and Compound A. In some embodiments, pembrolizumab is administered at least 6 times per week at an amount of about 440 mg, about 430 mg, about 420 mg, about 410 mg, about 400 mg, about 390 mg, about 380 mg, about 370 mg, about 360 mg, about 350 mg, about 340 mg, about 330 mg, about 320 mg, about 310 mg, about 300 mg, about 290 mg, about 280 mg, about 270 mg, about 260 mg, about 250 mg, about 240 mg, about 230 mg, about 220 mg, about 210 mg, or about 200 mg in combination with paclitaxel and Compound A. In some embodiments, pembrolizumab is administered at least 6 times per week at an amount of about 400 mg in combination with paclitaxel and Compound A. In some embodiments, pembrolizumab is administered at least 6 times per week at an amount of about 200 mg in combination with paclitaxel and Compound A.

In some embodiments, pembrolizumab is administered once every three weeks in combination with paclitaxel and Compound A.

In some embodiments, pembrolizumab is administered once every six weeks in combination with paclitaxel and Compound A.

In some embodiments, pembrolizumab is administered once every three weeks from about 1 mg to about 500 mg in combination with paclitaxel and Compound A.

In some embodiments, pembrolizumab is administered once every six weeks about 1 mg to about 500 mg in combination with paclitaxel and Compound A.

In some embodiments, pembrolizumab is administered once every three weeks from about 1 mg to about 400 mg in combination with paclitaxel and Compound A.

In some embodiments, pembrolizumab is administered once every six weeks from about 1 mg to about 400 mg in combination with paclitaxel and Compound A.

In some embodiments, pembrolizumab is administered once every three weeks from about 1 mg to about 300 mg in combination with paclitaxel and Compound A.

In some embodiments, pembrolizumab is administered once every six weeks from about 1 mg to about 300 mg in combination with paclitaxel and Compound A.

In some embodiments, pembrolizumab is administered once every three weeks from about 1 mg to about 200 mg in combination with paclitaxel and Compound A.

In some embodiments, pembrolizumab is administered once every six weeks from about 1 mg to about 200 mg in combination with paclitaxel and Compound A.

In some embodiments, pembrolizumab is administered once every three weeks from about 10 mg to about 500 mg in combination with paclitaxel and Compound A.

In some embodiments, pembrolizumab is administered once every six weeks from about 10 mg to about 500 mg in combination with paclitaxel and Compound A.

In some embodiments, pembrolizumab is administered once every three weeks from about 200 mg to about 500 mg in combination with paclitaxel and Compound A.

In some embodiments, pembrolizumab is administered once every six weeks from about 200 mg to about 500 mg in combination with paclitaxel and Compound A.

In some embodiments, pembrolizumab is administered once every three weeks at about 200 mg in combination with paclitaxel and Compound A.

In some embodiments, pembrolizumab is administered once every six weeks at about 200 mg in combination with paclitaxel and Compound A.

In some embodiments, pembrolizumab is administered once every three weeks at about 400 mg in combination with paclitaxel and Compound A.

In some embodiments, pembrolizumab is administered once every six weeks at about 400 mg in combination with paclitaxel and Compound A.

In some embodiments, the paclitaxel is administered in combination with Compound A and a checkpoint inhibitor, wherein Compound A is administered prior to paclitaxel.

In some embodiments, Compound A is administered at about 15 mg in combination with paclitaxel administered at about 270 mg and a checkpoint inhibitor at 200 mg, wherein Compound A is administered prior to paclitaxel.

In some embodiments, Compound A is administered at about 15 mg in combination with paclitaxel administered at about 270 mg and a checkpoint inhibitor at 400 mg, wherein Compound A is administered prior to paclitaxel.

In some embodiments, Compound A is administered at about 15 mg in combination with paclitaxel administered at about 270 mg and a checkpoint inhibitor at 200 mg, wherein Compound A is administered prior to paclitaxel, wherein the Compound A and paclitaxel are administered 3 days per week for two week of a three week cycle and the checkpoint inhibitor is administered once every three weeks.

In some embodiments, Compound A is administered at about 15 mg in combination with paclitaxel administered at about 270 mg and a checkpoint inhibitor at 400 mg, wherein Compound A is administered prior to paclitaxel, wherein the Compound A and paclitaxel are administered 3 days per week for two week of a three week cycle and the checkpoint inhibitor is administered once every six weeks.

In some embodiments, Compound A is administered at about 15 mg in combination with paclitaxel administered at about 300 mg and a checkpoint inhibitor at 200 mg, wherein Compound A is administered prior to paclitaxel.

In some embodiments, Compound A is administered at about 15 mg in combination with paclitaxel administered at about 300 mg and a checkpoint inhibitor at 400 mg, wherein Compound A is administered prior to paclitaxel.

In some embodiments, Compound A is administered at about 15 mg in combination with paclitaxel administered at about 300 mg and a checkpoint inhibitor at 200 mg, wherein Compound A is administered prior to paclitaxel, wherein the Compound A and paclitaxel are administered 3 days per week for two week of a three week cycle and the checkpoint inhibitor is administered once every three weeks.

In some embodiments, Compound A is administered at about 15 mg in combination with paclitaxel administered at about 300 mg and a checkpoint inhibitor at 400 mg, wherein Compound A is administered prior to paclitaxel, wherein the Compound A and paclitaxel are administered 3 days per week for two week of a three week cycle and the checkpoint inhibitor is administered once every six weeks.

In some embodiments, Compound A is administered at about 15 mg in combination with paclitaxel administered at about 330 mg and a checkpoint inhibitor at 200 mg, wherein Compound A is administered prior to paclitaxel.

In some embodiments, Compound A is administered at about 15 mg in combination with paclitaxel administered at about 330 mg and a checkpoint inhibitor at 400 mg, wherein Compound A is administered prior to paclitaxel.

In some embodiments, Compound A is administered at about 15 mg in combination with paclitaxel administered at about 330 mg and a checkpoint inhibitor at 200 mg, wherein Compound A is administered prior to paclitaxel, wherein the Compound A and paclitaxel are administered 3 days per week for two week of a three week cycle and the checkpoint inhibitor is administered once every three weeks.

In some embodiments, Compound A is administered at about 15 mg in combination with paclitaxel administered at about 330 mg and a checkpoint inhibitor at 400 mg, wherein Compound A is administered prior to paclitaxel, wherein the Compound A and paclitaxel are administered 3 days per week for two week of a three week cycle and the checkpoint inhibitor is administered once every six weeks.

In some embodiments, the paclitaxel, Compound A, and/or the checkpoint inhibitor are administered at the various doses and dosing frequencies described herein for a duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks.

In some embodiments, the paclitaxel, Compound A, and/or the checkpoint inhibitor are administered at the various doses and dosing frequencies described herein for a duration of between 1 week and 2 weeks, between 1 week and 3 weeks, between 1 week and 4 weeks, between 1 week and 5 weeks, between 1 week and 6 weeks, between 1 week and 7 weeks, between 1 week and 8 weeks, between 1 week and 9 weeks, between 1 week and 10 weeks, between 1 week and 11 weeks, between 1 week and 12 weeks, between 1 week and 13 weeks, between 1 week and 14 weeks, between 1 week and 15 weeks, between 1 week and 16 weeks, between 1 week and 17 weeks, between 1 week and 18 weeks, between 1 week and 19 weeks, between 1 week and 20 weeks, between 1 week and 21 weeks, between 1 week and 22 weeks, between 1 week and 23 weeks, between 1 week and 24 weeks, between 1 week and 25 weeks, between 1 week and 26 weeks, between 1 week and 27 weeks, between 1 week and 28 weeks, between 1 week and 29 weeks, or between 1 week and 30 weeks.

In some embodiments, the paclitaxel, Compound A, and/or the checkpoint inhibitor are administered at the various doses and dosing frequencies described herein for a duration of between 2 weeks and 3 weeks, between 2 weeks and 4 weeks, between 2 weeks and 5 weeks, between 2 weeks and 6 weeks, between 2 weeks and 7 weeks, between 2 weeks and 8 weeks, between 2 weeks and 9 weeks, between 2 weeks and 10 weeks, between 2 weeks and 11 weeks, between 2 weeks and 12 weeks, between 2 weeks and 13 weeks, between 2 weeks and 14 weeks, between 2 weeks and 15 weeks, between 2 weeks and 16 weeks, between 2 weeks and 17 weeks, between 2 weeks and 18 weeks, between 2 weeks and 19 weeks, between 2 weeks and 20 weeks, between 2 weeks and 21 weeks, between 2 weeks and 22 weeks, between 2 weeks and 23 weeks, between 2 weeks and 24 weeks, between 2 weeks and 25 weeks, between 2 weeks and 26 weeks, between 2 weeks and 27 weeks, between 2 weeks and 28 weeks, between 2 weeks and 29 weeks, or between 2 weeks and 30 weeks.

In some embodiments, the paclitaxel, Compound A, and/or the checkpoint inhibitor are administered at the various doses and dosing frequencies described herein for a duration of between 3 weeks and 4 weeks, between 3 weeks and 5 weeks, between 3 weeks and 6 weeks, between 3 weeks and 7 weeks, between 3 weeks and 8 weeks, between 3 weeks and 9 weeks, between 3 weeks and 10 weeks, between 3 weeks and 11 weeks, between 3 weeks and 12 weeks, between 3 weeks and 13 weeks, between 3 weeks and 14 weeks, between 3 weeks and 15 weeks, between 3 weeks and 16 weeks, between 3 weeks and 17 weeks, between 3 weeks and 18 weeks, between 3 weeks and 19 weeks, between 3 weeks and 20 weeks, between 3 weeks and 21 weeks, between 3 weeks and 22 weeks, between 3 weeks and 23 weeks, between 3 weeks and 24 weeks, between 3 weeks and 25 weeks, between 3 weeks and 26 weeks, between 3 weeks and 27 weeks, between 3 weeks and 28 weeks, between 3 weeks and 29 weeks, or between 3 weeks and 30 weeks.

In some embodiments, the paclitaxel, Compound A, and/or the checkpoint inhibitor are administered at the various doses and dosing frequencies described herein for a duration of between 4 weeks and 5 weeks, between 4 weeks and 6 weeks, between 4 weeks and 7 weeks, between 4 weeks and 8 weeks, between 4 weeks and 9 weeks, between 4 weeks and 10 weeks, between 4 weeks and 11 weeks, between 4 weeks and 12 weeks, between 4 weeks and 13 weeks, between 4 weeks and 14 weeks, between 4 weeks and 15 weeks, between 4 weeks and 16 weeks, between 4 weeks and 17 weeks, between 4 weeks and 18 weeks, between 4 weeks and 19 weeks, between 4 weeks and 20 weeks, between 4 weeks and 21 weeks, between 4 weeks and 22 weeks, between 4 weeks and 23 weeks, between 4 weeks and 24 weeks, between 4 weeks and 25 weeks, between 4 weeks and 26 weeks, between 4 weeks and 27 weeks, between 4 weeks and 28 weeks, between 4 weeks and 29 weeks, or between 4 weeks and 30 weeks.

In some embodiments, the paclitaxel, Compound A, and/or the checkpoint inhibitor are administered at the various doses and dosing frequencies described herein for a duration of between 5 weeks and 6 weeks, between 5 weeks and 7 weeks, between 5 weeks and 8 weeks, between 5 weeks and 9 weeks, between 5 weeks and 10 weeks, between 5 weeks and 11 weeks, between 5 weeks and 12 weeks, between 5 weeks and 13 weeks, between 5 weeks and 14 weeks, between 5 weeks and 15 weeks, between 5 weeks and 16 weeks, between 5 weeks and 17 weeks, between 5 weeks and 18 weeks, between 5 weeks and 19 weeks, between 5 weeks and 20 weeks, between 5 weeks and 21 weeks, between 5 weeks and 22 weeks, between 5 weeks and 23 weeks, between 5 weeks and 24 weeks, between 5 weeks and 25 weeks, between 5 weeks and 26 weeks, between 5 weeks and 27 weeks, between 5 weeks and 28 weeks, between 5 weeks and 29 weeks, or between 5 weeks and 30 weeks.

In some embodiments, the paclitaxel, Compound A, and/or the checkpoint inhibitor are administered at the various doses and dosing frequencies described herein for a duration of between 6 weeks and 7 weeks, between 6 weeks and 8 weeks, between 6 weeks and 9 weeks, between 6 weeks and 10 weeks, between 6 weeks and 11 weeks, between 6 weeks and 12 weeks, between 6 weeks and 13 weeks, between 6 weeks and 14 weeks, between 6 weeks and 15 weeks, between 6 weeks and 16 weeks, between 6 weeks and 17 weeks, between 6 weeks and 18 weeks, between 6 weeks and 19 weeks, between 6 weeks and 20 weeks, between 6 weeks and 21 weeks, between 6 weeks and 22 weeks, between 6 weeks and 23 weeks, between 6 weeks and 24 weeks, between 6 weeks and 25 weeks, between 6 weeks and 26 weeks, between 6 weeks and 27 weeks, between 6 weeks and 28 weeks, between 6 weeks and 29 weeks, or between 6 weeks and 30 weeks.

In some embodiments, the paclitaxel, Compound A, and/or the checkpoint inhibitor are administered at the various doses and dosing frequencies described herein for a duration of between 7 weeks and 8 weeks, between 7 weeks and 9 weeks, between 7 weeks and 10 weeks, between 7 weeks and 11 weeks, between 7 weeks and 12 weeks, between 7 weeks and 13 weeks, between 7 weeks and 14 weeks, between 7 weeks and 15 weeks, between 7 weeks and 16 weeks, between 7 weeks and 17 weeks, between 7 weeks and 18 weeks, between 7 weeks and 19 weeks, between 7 weeks and 20 weeks, between 7 weeks and 21 weeks, between 7 weeks and 22 weeks, between 7 weeks and 23 weeks, between 7 weeks and 24 weeks, between 7 weeks and 25 weeks, between 7 weeks and 26 weeks, between 7 weeks and 27 weeks, between 7 weeks and 28 weeks, between 7 weeks and 29 weeks, or between 7 weeks and 30 weeks.

In some embodiments, the paclitaxel, Compound A, and/or the checkpoint inhibitor are administered at the various doses and dosing frequencies described herein for a duration of between 8 weeks and 9 weeks, between 8 weeks and 10 weeks, between 8 weeks and 11 weeks, between 8 weeks and 12 weeks, between 8 weeks and 13 weeks, between 8 weeks and 14 weeks, between 8 weeks and 15 weeks, between 8 weeks and 16 weeks, between 8 weeks and 17 weeks, between 8 weeks and 18 weeks, between 8 weeks and 19 weeks, between 8 weeks and 20 weeks, between 8 weeks and 21 weeks, between 8 weeks and 22 weeks, between 8 weeks and 23 weeks, between 8 weeks and 24 weeks, between 8 weeks and 25 weeks, between 8 weeks and 26 weeks, between 8 weeks and 27 weeks, between 8 weeks and 28 weeks, between 8 weeks and 29 weeks, or between 8 weeks and 30 weeks.

In some embodiments, the paclitaxel, Compound A, and/or the checkpoint inhibitor are administered at the various doses and dosing frequencies described herein for a duration of between 9 weeks and 10 weeks, between 9 weeks and 11 weeks, between 9 weeks and 12 weeks, between 9 weeks and 13 weeks, between 9 weeks and 14 weeks, between 9 weeks and 15 weeks, between 9 weeks and 16 weeks, between 9 weeks and 17 weeks, between 9 weeks and 18 weeks, between 9 weeks and 19 weeks, between 9 weeks and 20 weeks, between 9 weeks and 21 weeks, between 9 weeks and 22 weeks, between 9 weeks and 23 weeks, between 9 weeks and 24 weeks, between 9 weeks and 25 weeks, between 9 weeks and 26 weeks, between 9 weeks and 27 weeks, between 9 weeks and 28 weeks, between 9 weeks and 29 weeks, or between 9 weeks and 30 weeks.

In some embodiments, the paclitaxel, Compound A, and/or the checkpoint inhibitor are administered at the various doses and dosing frequencies described herein for a duration of between 10 weeks and 11 weeks, between 10 weeks and 12 weeks, between 10 weeks and 13 weeks, between 10 weeks and 14 weeks, between 10 weeks and 15 weeks, between 10 weeks and 16 weeks, between 10 weeks and 17 weeks, between 10 weeks and 18 weeks, between 10 weeks and 19 weeks, between 10 weeks and 20 weeks, between 10 weeks and 21 weeks, between 10 weeks and 22 weeks, between 10 weeks and 23 weeks, between 10 weeks and 24 weeks, between 10 weeks and 25 weeks, between 10 weeks and 26 weeks, between 10 weeks and 27 weeks, between 10 weeks and 28 weeks, between 10 weeks and 29 weeks, or between 10 weeks and 30 weeks.

In some embodiments, the paclitaxel, Compound A, and/or the checkpoint inhibitor are administered at the various doses and dosing frequencies described herein for a duration of between 11 weeks and 12 weeks, between 11 weeks and 13 weeks, between 11 weeks and 14 weeks, between 11 weeks and 15 weeks, between 11 weeks and 16 weeks, between 11 weeks and 17 weeks, between 11 weeks and 18 weeks, between 11 weeks and 19 weeks, between 11 weeks and 20 weeks, between 11 weeks and 21 weeks, between 11 weeks and 22 weeks, between 11 weeks and 23 weeks, between 11 weeks and 24 weeks, between 11 weeks and 25 weeks, between 11 weeks and 26 weeks, between 11 weeks and 27 weeks, between 11 weeks and 28 weeks, between 11 weeks and 29 weeks, or between 11 weeks and 30 weeks.

In some embodiments, the paclitaxel, Compound A, and/or the checkpoint inhibitor are administered at the various doses and dosing frequencies described herein for a duration of between 12 weeks and 13 weeks, between 12 weeks and 14 weeks, between 12 weeks and 15 weeks, between 12 weeks and 16 weeks, between 12 weeks and 17 weeks, between 12 weeks and 18 weeks, between 12 weeks and 19 weeks, between 12 weeks and 20 weeks, between 12 weeks and 21 weeks, between 12 weeks and 22 weeks, between 12 weeks and 23 weeks, between 12 weeks and 24 weeks, between 12 weeks and 25 weeks, between 12 weeks and 26 weeks, between 12 weeks and 27 weeks, between 12 weeks and 28 weeks, between 12 weeks and 29 weeks, or between 12 weeks and 30 weeks.

In some embodiments, the paclitaxel, Compound A, and/or the checkpoint inhibitor are administered at the various doses and dosing frequencies described herein for a duration of between 13 weeks and 14 weeks, between 13 weeks and 15 weeks, between 13 weeks and 16 weeks, between 13 weeks and 17 weeks, between 13 weeks and 18 weeks, between 13 weeks and 19 weeks, between 13 weeks and 20 weeks, between 13 weeks and 21 weeks, between 13 weeks and 22 weeks, between 13 weeks and 23 weeks, between 13 weeks and 24 weeks, between 13 weeks and 25 weeks, between 13 weeks and 26 weeks, between 13 weeks and 27 weeks, between 13 weeks and 28 weeks, between 13 weeks and 29 weeks, or between 13 weeks and 30 weeks.

In some embodiments, the paclitaxel, Compound A, and/or the checkpoint inhibitor are administered at the various doses and dosing frequencies described herein for a duration of between 14 weeks and 15 weeks, between 14 weeks and 16 weeks, between 14 weeks and 17 weeks, between 14 weeks and 18 weeks, between 14 weeks and 19 weeks, between 14 weeks and 20 weeks, between 14 weeks and 21 weeks, between 14 weeks and 22 weeks, between 14 weeks and 23 weeks, between 14 weeks and 24 weeks, between 14 weeks and 25 weeks, between 14 weeks and 26 weeks, between 14 weeks and 27 weeks, between 14 weeks and 28 weeks, between 14 weeks and 29 weeks, or between 14 weeks and 30 weeks.

In some embodiments, the paclitaxel, Compound A, and/or the checkpoint inhibitor are administered at the various doses and dosing frequencies described herein for a duration of between 15 weeks and 16 weeks, between 15 weeks and 17 weeks, between 15 weeks and 18 weeks, between 15 weeks and 19 weeks, between 15 weeks and 20 weeks, between 15 weeks and 21 weeks, between 15 weeks and 22 weeks, between 15 weeks and 23 weeks, between 15 weeks and 24 weeks, between 15 weeks and 25 weeks, between 15 weeks and 26 weeks, between 15 weeks and 27 weeks, between 15 weeks and 28 weeks, between 15 weeks and 29 weeks, or between 15 weeks and 30 weeks.

In some embodiments, the paclitaxel, Compound A, and/or the checkpoint inhibitor are administered at the various doses and dosing frequencies described herein for a duration of between 16 weeks and 17 weeks, between 16 weeks and 18 weeks, between 16 weeks and 19 weeks, between 16 weeks and 20 weeks, between 16 weeks and 21 weeks, between 16 weeks and 22 weeks, between 16 weeks and 23 weeks, between 16 weeks and 24 weeks, between 16 weeks and 25 weeks, between 16 weeks and 26 weeks, between 16 weeks and 27 weeks, between 16 weeks and 28 weeks, between 16 weeks and 29 weeks, or between 16 weeks and 30 weeks.

In some embodiments, the paclitaxel, Compound A, and/or the checkpoint inhibitor are administered at the various doses and dosing frequencies described herein for a duration of between 17 weeks and 18 weeks, between 17 weeks and 19 weeks, between 17 weeks and 20 weeks, between 17 weeks and 21 weeks, between 17 weeks and 22 weeks, between 17 weeks and 23 weeks, between 17 weeks and 24 weeks, between 17 weeks and 25 weeks, between 17 weeks and 26 weeks, between 17 weeks and 27 weeks, between 17 weeks and 28 weeks, between 17 weeks and 29 weeks, or between 17 weeks and 30 weeks.

In some embodiments, the paclitaxel, Compound A, and/or the checkpoint inhibitor are administered at the various doses and dosing frequencies described herein for a duration of between 18 weeks and 19 weeks, between 18 weeks and 20 weeks, between 18 weeks and 21 weeks, between 18 weeks and 22 weeks, between 18 weeks and 23 weeks, between 18 weeks and 24 weeks, between 18 weeks and 25 weeks, between 18 weeks and 26 weeks, between 18 weeks and 27 weeks, between 18 weeks and 28 weeks, between 18 weeks and 29 weeks, or between 18 weeks and 30 weeks.

In some embodiments, the paclitaxel, Compound A, and/or the checkpoint inhibitor are administered at the various doses and dosing frequencies described herein for a duration of between 19 weeks and 20 weeks, between 19 weeks and 21 weeks, between 19 weeks and 22 weeks, between 19 weeks and 23 weeks, between 19 weeks and 24 weeks, between 19 weeks and 25 weeks, between 19 weeks and 26 weeks, between 19 weeks and 27 weeks, between 19 weeks and 28 weeks, between 19 weeks and 29 weeks, or between 19 weeks and 30 weeks.

In some embodiments, the paclitaxel, Compound A, and/or the checkpoint inhibitor are administered at the various doses and dosing frequencies described herein for a duration of between 20 weeks and 21 weeks, between 20 weeks and 22 weeks, between 20 weeks and 23 weeks, between 20 weeks and 24 weeks, between 20 weeks and 25 weeks, between 20 weeks and 26 weeks, between 20 weeks and 27 weeks, between 20 weeks and 28 weeks, between 20 weeks and 29 weeks, or between 20 weeks and 30 weeks.

In some embodiments, the paclitaxel, Compound A, and/or the checkpoint inhibitor are administered at the various doses and dosing frequencies described herein for a duration of 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the initial duration of administration of the paclitaxel, Compound A, and/or the checkpoint inhibitor at the various doses and dosing frequencies described herein, the paclitaxel, Compound A, and/or the checkpoint inhibitor are administered for a second duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration and the second duration is at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration and the second duration is 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the initial duration and the second duration of administration of the paclitaxel, Compound A, and/or the checkpoint inhibitor at the various doses and dosing frequencies described herein, the paclitaxel, Compound A, and/or the checkpoint inhibitor are administered for a third duration of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration, the second duration, and the third duration is at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks. In some embodiments, the total duration of the initial duration, the second duration, and the third duration is 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, or 30 weeks.

In some embodiments, after the paclitaxel, Compound A, and/or the checkpoint inhibitor are each administered at a first dose described herein and at dosing frequencies described herein for a first duration described herein (e.g., at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, or at least 30 weeks), the paclitaxel, Compound A, and/or the checkpoint inhibitor are each administered at a second dose described herein and at dosing frequencies described herein for a second duration described herein. In some embodiments, the second dose for the paclitaxel, Compound A, and/or the checkpoint inhibitor is the same as the first dose for the paclitaxel, Compound A, and/or the checkpoint inhibitor. In some embodiments, the second dose for the paclitaxel, Compound A, and/or the checkpoint inhibitor is different from the first dose for the paclitaxel, Compound A, and/or the checkpoint inhibitor. In some embodiments, the second dose for the paclitaxel, Compound A, and/or the checkpoint inhibitor is reduced from the first dose for the paclitaxel, Compound A, and/or the checkpoint inhibitor (e.g., for paclitaxel, the second dose is about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose).

In some embodiments, after the paclitaxel, Compound A, and/or the checkpoint inhibitor are each administered at a first dose described herein and at dosing frequencies described herein for a first duration described herein, followed by a second dose described herein and at dosing frequencies described herein for a second duration described herein, the paclitaxel, Compound A, and/or the checkpoint inhibitor are each administered at a third dose described herein and at dosing frequencies described herein for a third duration described herein. In some embodiments, the third dose for the paclitaxel, Compound A, and/or the checkpoint inhibitor is the same as the second dose for the paclitaxel, Compound A, and/or the checkpoint inhibitor. In some embodiments, the third dose for the paclitaxel, Compound A, and/or the checkpoint inhibitor is different from the second dose for the paclitaxel, Compound A, and/or the checkpoint inhibitor.

In some embodiments, the third dose for the paclitaxel, Compound A, and/or the checkpoint inhibitor is reduced from the second dose for the paclitaxel, Compound A, and/or the checkpoint inhibitor (e.g., for paclitaxel, the second dose is about 5 mg/m², about 10 mg/m², about 15 mg/m², about 20 mg/m², about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², or about 100 mg/m² less than the first dose).

In some embodiments, the paclitaxel, Compound A, and/or the checkpoint inhibitor are each administered for a duration until the subject is treated. In some embodiments, the subject is treated when there is no disease progression (e.g., the cancer is not growing in size and/or severity, and not metastasizing). In some embodiments, the subject is treated when the disease is reversed (e.g., the cancer is reducing size and/or severity).

In some embodiments, the paclitaxel, Compound A, and/or the checkpoint inhibitor are each administered for a duration until the subject displays one or more adverse reactions. In some embodiments, the paclitaxel, Compound A, and/or the checkpoint inhibitor are each administered for a duration until the subject displays disease progression (e.g., the cancer is growing in size and/or severity, and/or the cancer is metastasizing).

In some embodiments, the paclitaxel is administered in capsules. In some embodiments, the paclitaxel is administered in 10 mg capsules. In some embodiments, the paclitaxel is administered in 15 mg capsules. In some embodiments, the paclitaxel is administered in 20 mg capsules. In some embodiments, the paclitaxel is administered in 25 mg capsules. In some embodiments, the paclitaxel is administered in 30 mg capsules. In some embodiments, the paclitaxel is administered in 35 mg capsules. In some embodiments, the paclitaxel is administered in 40 mg capsules. In some embodiments, the paclitaxel is administered in 45 mg capsules. In some embodiments, the paclitaxel is administered in 50 mg capsules.

In some embodiments, Compound A and the paclitaxel are administered on the same day.

In some embodiments, Compound A is administered before the paclitaxel is administered.

In some embodiments, Compound A is administered about 5 minutes before the paclitaxel is administered.

In some embodiments, Compound A is administered about 10 minutes before the paclitaxel is administered.

In some embodiments, Compound A is administered about 15 minutes before the paclitaxel is administered.

In some embodiments, Compound A is administered about 30 minutes before the paclitaxel is administered.

In some embodiments, Compound A is administered about 45 minutes before the paclitaxel is administered.

In some embodiments, Compound A is administered about 60 minutes before the paclitaxel is administered.

In some embodiments, Compound A is administered about 65 minutes before the paclitaxel is administered.

In some embodiments, Compound A is administered about 70 minutes before the paclitaxel is administered.

In some embodiments, Compound A is administered about 75 minutes before the paclitaxel is administered.

In some embodiments, Compound A is administered about 80 minutes before the paclitaxel is administered.

In some embodiments, Compound A is administered about 85 minutes before the paclitaxel is administered.

In some embodiments, Compound A is administered about 90 minutes before the paclitaxel is administered.

In some embodiments, Compound A is administered about 2 hours before the paclitaxel is administered.

In some embodiments, Compound A is administered about 3 hours before the paclitaxel is administered.

In some embodiments, Compound A is administered about 4 hours before the paclitaxel is administered.

In some embodiments, Compound A is administered about 6 hours before the paclitaxel is administered.

In some embodiments, Compound A is administered about 8 hours before the paclitaxel is administered.

In some embodiments, about 15 mg of Compound A is administered before the paclitaxel is administered.

In some embodiments, about 15 mg of Compound A is administered about 5 minutes before the paclitaxel is administered.

In some embodiments, about 15 mg of Compound A is administered about 10 minutes before the paclitaxel is administered.

In some embodiments, about 15 mg of Compound A is administered about 15 minutes before the paclitaxel is administered.

In some embodiments, about 15 mg of Compound A is administered about 30 minutes before the paclitaxel is administered.

In some embodiments, about 15 mg of Compound A is administered about 45 minutes before the paclitaxel is administered.

In some embodiments, about 15 mg of Compound A is administered about 60 minutes before the paclitaxel is administered.

In some embodiments, about 15 mg of Compound A is administered about 65 minutes before the paclitaxel is administered.

In some embodiments, about 15 mg of Compound A is administered about 70 minutes before the paclitaxel is administered.

In some embodiments, about 15 mg of Compound A is administered about 75 minutes before the paclitaxel is administered.

In some embodiments, about 15 mg of Compound A is administered about 80 minutes before the paclitaxel is administered.

In some embodiments, about 15 mg of Compound A is administered about 85 minutes before the paclitaxel is administered.

In some embodiments, about 15 mg of Compound A is administered about 90 minutes before the paclitaxel is administered.

In some embodiments, about 15 mg of Compound A is administered about 2 hours before the paclitaxel is administered.

In some embodiments, about 15 mg of Compound A is administered about 3 hours before the paclitaxel is administered.

In some embodiments, about 15 mg of Compound A is administered about 4 hours before the paclitaxel is administered.

In some embodiments, about 15 mg of Compound A is administered about 6 hours before the paclitaxel is administered.

In some embodiments, about 15 mg of Compound A is administered about 8 hours before the paclitaxel is administered.

In some embodiments, an anti-emetic is administered. In some embodiments, the anti-emetic is administered simultaneously with or prior to Compound A. In some embodiments, the anti-emetic is administered simultaneously with Compound A. In some embodiments, the anti-emetic is administered about 5 minutes before Compound A is administered. In some embodiments, the anti-emetic is administered about 10 minutes before Compound A is administered. In some embodiments, the anti-emetic is administered about 15 minutes before Compound A is administered. In some embodiments, the anti-emetic is administered about 30 minutes before Compound A is administered. In some embodiments, the anti-emetic is administered about 45 minutes before Compound A is administered. In some embodiments, the anti-emetic is administered about 60 minutes before Compound A is administered.

In some embodiments, the anti-emetic is aprepitant. In some embodiments, the anti-emetic is dexamethasone. In some embodiments, the anti-emetic is dolasetron. In some embodiments, the anti-emetic is granisetron. In some embodiments, the anti-emetic is ondansetron. In some embodiments, the anti-emetic is palonosetron. In some embodiments, the anti-emetic is prochlorperazine. In some embodiments, the anti-emetic is rolapitant. In some embodiments, the anti-emetic is droperidol. In some embodiments, the anti-emetic is metoclopramide. In some embodiments, the anti-emetic is dimenhydrinate. In some embodiments, the anti-emetic is diphenhydramine. In some embodiments, the anti-emetic is meclizine. In some embodiments, the anti-emetic is promethazine.

In some embodiments, a 5-hydroxytryptamine (5-HT3) antagonist is administered. In some embodiments, a 5-HT3 antagonist is administered simultaneously with Compound A. In some embodiments, a 5-HT3 antagonist is administered about 5 minutes before Compound A is administered. In some embodiments, a 5-HT3 antagonist is administered about 10 minutes before Compound A is administered. In some embodiments, a 5-HT3 antagonist is administered about 15 minutes before Compound A is administered. In some embodiments, a 5-HT3 antagonist is administered about 30 minutes before Compound A is administered. In some embodiments, a 5-HT3 antagonist is administered about 45 minutes before Compound A is administered. In some embodiments, a 5-HT3 antagonist is administered about 60 minutes before Compound A is administered. In some embodiment, 5-hydroxytryptamine (5-HT3) antagonist is administered for prophylaxis. In some embodiment, 5-hydroxytryptamine (5-HT3) antagonist is administered for reducing nausea. In some embodiment, a 5-hydroxytryptamine (5-HT3) antagonist is administered for reducing vomiting.

In some embodiments, a steroid antagonist is administered. In some embodiments, a steroid is administered simultaneously with Compound A. In some embodiments, a steroid is administered about 5 minutes before Compound A is administered. In some embodiments, a steroid is administered about 10 minutes before Compound A is administered. In some embodiments, a steroid is administered about 15 minutes before Compound A is administered. In some embodiments, a steroid is administered about 30 minutes before Compound A is administered. In some embodiments, a steroid is administered about 45 minutes before Compound A is administered. In some embodiments, a steroid is administered about 60 minutes before Compound A is administered. In some embodiment, a steroid is administered for reducing hypersensitivity-type reactions.

In some embodiments, a H-1 receptor antagonist is administered. In some embodiments, a H-1 receptor antagonist is administered at the same time as Compound A. In some embodiments, a H-1 receptor antagonist is administered simultaneously with Compound A. In some embodiments, a H-1 receptor antagonist is administered about 5 minutes before Compound A is administered. In some embodiments, a H-1 receptor antagonist is administered about 10 minutes before Compound A is administered. In some embodiments, a H-1 receptor antagonist is administered about 15 minutes before Compound A is administered. In some embodiments, a H-1 receptor antagonist is administered about 30 minutes before Compound A is administered. In some embodiments, a H-1 receptor antagonist is administered about 45 minutes before Compound A is administered. In some embodiments, a H-1 receptor antagonist is administered about 60 minutes before Compound A is administered. In some embodiment, a H-1 receptor antagonist is administered for reducing hypersensitivity-type reactions.

In some embodiments, a potential drug interaction exists between Compound A and moderate to strong inhibitors or inducers of CYP3A4 or CYP2C8. In some embodiments, a subject is not taking a medication which is a moderate to strong inhibitor or inducer of CYP3A4.

In some embodiments, a subject is not taking a medication which is a moderate to strong inhibitor or inducer of CYP2C8. In some embodiments, a potential drug interaction exists between Compound A and inhibitors or inducers of P-gp. In some embodiments, a subject is not taking a medication which is an inhibitor or inducer of P-gp.

In some embodiments, a subject is not administered a medication known to be a strong P-gp inhibitor or inducer within 14 days before the oral administration of paclitaxel. In some embodiments, a subject is not administered an oral medication with a narrow therapeutic index known to be a P-gp substrate (e.g., dabigatran and digoxin) within 24 hours before the oral administration of paclitaxel. In some embodiments, a subject is not administered a medication known to be a strong CYP3A4 inhibitor (e.g., ketoconazole) or inducer (e.g., rifampin or St. John's Wort) within 14 days before the oral administration of paclitaxel. In some embodiments, a subject is not administered a medication known to be a strong inhibitor (e.g., gemfibrozil) or inducers (e.g., rifampin) of CYP2C8 within 14 days before the oral administration of paclitaxel.

In some embodiments, the AUC_{(0→24)} of the orally administered paclitaxel is about 100 ng·h/mL to about 10,000 ng·h/mL.

In some embodiments, the AUC_{(0→24)} of the orally administered paclitaxel is about 100 ng·h/mL to about 1,000 ng·h/mL.

In some embodiments, the AUC_{(0→24)} of the orally administered paclitaxel is about 100 ng·h/mL to about 900 ng·h/mL.

In some embodiments, the AUC_{(0→24)} of the orally administered paclitaxel is about 100 ng·h/mL to about 800 ng·h/mL.

In some embodiments, the AUC_{(0→24)} of the orally administered paclitaxel is about 100 ng·h/mL to about 700 ng·h/mL.

In some embodiments, the AUC_{(0→24)} of the orally administered paclitaxel is about 100 ng·h/mL to about 600 ng·h/mL.

In some embodiments, the AUC_{(0→24)} of the orally administered paclitaxel is about 100 ng·h/mL to about 500 ng·h/mL.

In some embodiments, the AUC_{(0→24)} of the orally administered paclitaxel is about 100 ng·h/mL to about 400 ng·h/mL.

In some embodiments, the AUC_{(0→24)} of the orally administered paclitaxel is about 100 ng·h/mL to about 300 ng·h/mL.

In some embodiments, the AUC_{(0→24)} of the orally administered paclitaxel is about 100 ng·h/mL to about 200 ng·h/mL.

In some embodiments, the AUC_{(0→24)} of the orally administered paclitaxel is about 200 ng·h/mL to about 1,000 ng·h/mL.

In some embodiments, the AUC_{(0→24)} of the orally administered paclitaxel is about 300 ng·h/mL to about 1,000 ng·h/mL.

In some embodiments, the AUC_{(0→24)} of the orally administered paclitaxel is about 400 ng·h/mL to about 1,000 ng·h/mL.

In some embodiments, the AUC_{(0→24)} of the orally administered paclitaxel is about 500 ng·h/mL to about 1,000 ng·h/mL.

In some embodiments, the AUC_{(0→24)} of the orally administered paclitaxel is about 600 ng·h/mL to about 1,000 ng·h/mL.

In some embodiments, the AUC_{(0→24)} of the orally administered paclitaxel is about 700 ng·h/mL to about 1,000 ng·h/mL.

In some embodiments, the AUC_{(0→24)} of the orally administered paclitaxel is about 800 ng·h/mL to about 1,000 ng·h/mL.

In some embodiments, the AUC_{(0→24)} of the orally administered paclitaxel is about 900 ng·h/mL to about 1,000 ng·h/mL.

In some embodiments, the AUC_{(0→24)} of the orally administered paclitaxel is about 100 ng·h/mL.

In some embodiments, the AUC_{(0→24)} of the orally administered paclitaxel is about 200 ng·h/mL.

In some embodiments, the AUC_{(0→24)} of the orally administered paclitaxel is about 300 ng·h/mL.

In some embodiments, the AUC_{(0→24)} of the orally administered paclitaxel is about 400 ng·h/mL.

In some embodiments, the AUC_{(0→24)} of the orally administered paclitaxel is about 500 ng·h/mL.

In some embodiments, the AUC_{(0→24)} of the orally administered paclitaxel is about 600 ng·h/mL.

In some embodiments, the AUC_{(0→24)} of the orally administered paclitaxel is about 700 ng·h/mL.

In some embodiments, the AUC_{(0→24)} of the orally administered paclitaxel is about 800 ng·h/mL.

In some embodiments, the AUC_{(0→24)} of the orally administered paclitaxel is about 900 ng·h/mL.

In some embodiments, the AUC_{(0→24)} of the orally administered paclitaxel is about 1,000 ng·h/mL.

In some embodiments, the AUC_{(0→24)} of the orally administered paclitaxel is about 2,000 ng·h/mL.

In some embodiments, the AUC_{(0→24)} of the orally administered paclitaxel is about 3,000 ng·h/mL.

In some embodiments, the AUC_{(0→24)} of the orally administered paclitaxel is about 4,000 ng·h/mL.

In some embodiments, the AUC_{(0→24)} of the orally administered paclitaxel is about 5,000 ng·h/mL.

In some embodiments, the AUC_{(0→24)} of the orally administered paclitaxel is about 6,000 ng·h/mL.

In some embodiments, the AUC_{(0→24)} of the orally administered paclitaxel is about 7,000 ng·h/mL.

In some embodiments, the AUC_{(0→24)} of the orally administered paclitaxel is about 8,000 ng·h/mL.

In some embodiments, the AUC_{(0→24)} of the orally administered paclitaxel is about 9,000 ng·h/mL.

In some embodiments, the AUC_{(0→24)} of the orally administered paclitaxel is about 10,000 ng·h/mL.

### Methods of Use

In some aspects, the present disclosure provides a method of treating or preventing a disease or disorder disclosed herein in a subject in need thereof, comprising administering to the subject a therapeutically effective amount of a combination of the present disclosure or a pharmaceutical composition of the present disclosure.

In some aspects, the present disclosure provides a method of treating a disease or disorder disclosed herein in a subject in need thereof, comprising administering to the subject a therapeutically effective amount of a combination of the present disclosure or a pharmaceutical composition of the present disclosure.

In some embodiments, the disease or disorder is cancer. In some embodiments, the disease or disorder is a solid tumor. In some embodiments, the cancer is lung cancer. In some embodiments, the cancer is non-small cell lung cancer.

In some aspects, the present disclosure provides a method of treating or preventing cancer in a subject in need thereof, comprising administering to the subject a therapeutically effective amount of a combination of the present disclosure or a pharmaceutical composition of the present disclosure.

In some aspects, the present disclosure provides a method of treating cancer in a subject in need thereof, comprising administering to the subject a therapeutically effective amount of a combination of the present disclosure or a pharmaceutical composition of the present disclosure.

In some aspects, the present disclosure provides a method of treating or preventing lung cancer in a subject in need thereof, comprising administering to the subject a therapeutically effective amount of a combination of the present disclosure or a pharmaceutical composition of the present disclosure.

In some aspects, the present disclosure provides a method of treating lung cancer in a subject in need thereof, comprising administering to the subject a therapeutically effective amount of a combination of the present disclosure or a pharmaceutical composition of the present disclosure.

In some aspects, the present disclosure provides a method of treating or preventing non-small cell lung cancer in a subject in need thereof, comprising administering to the subject a therapeutically effective amount of a combination of the present disclosure or a pharmaceutical composition of the present disclosure.

In some aspects, the present disclosure provides a method of treating non-small cell lung cancer in a subject in need thereof, comprising administering to the subject a therapeutically effective amount of a combination of the present disclosure or a pharmaceutical composition of the present disclosure.

In some aspects, the present disclosure provides a combination of the present disclosure or a pharmaceutical composition thereof for use in treating or preventing a disease or disorder disclosed herein.

In some aspects, the present disclosure provides a combination of the present disclosure or a pharmaceutical composition thereof for use in treating a disease or disorder disclosed herein.

In some aspects, the present disclosure provides a combination of the present disclosure or a pharmaceutical composition thereof for use in treating or preventing a cancer.

In some aspects, the present disclosure provides a combination of the present disclosure or a pharmaceutical composition thereof for use in treating a cancer.

In some aspects, the present disclosure provides a combination of the present disclosure or a pharmaceutical composition thereof for use in treating or preventing lung cancer.

In some aspects, the present disclosure provides a combination of the present disclosure or a pharmaceutical composition thereof for use in treating lung cancer.

In some aspects, the present disclosure provides a combination of the present disclosure or a pharmaceutical composition thereof for use in treating or preventing non-small cell lung cancer.

In some aspects, the present disclosure provides a combination of the present disclosure or a pharmaceutical composition thereof for use in treating non-small cell lung cancer.

In some aspects, the present disclosure provides use of a combination of the present disclosure or a pharmaceutical composition thereof in the manufacture of a medicament for treating or preventing a disease or disorder disclosed herein.

In some aspects, the present disclosure provides use of a combination of the present disclosure or a pharmaceutical composition thereof in the manufacture of a medicament for treating a disease or disorder disclosed herein.

In some aspects, the present disclosure provides use of a combination of the present disclosure or a pharmaceutical composition thereof in the manufacture of a medicament for treating or preventing a cancer.

In some aspects, the present disclosure provides use of a combination of the present disclosure or a pharmaceutical composition thereof in the manufacture of a medicament for treating a cancer.

In some aspects, the present disclosure provides use of a combination of the present disclosure or a pharmaceutical composition thereof in the manufacture of a medicament for treating or preventing lung cancer.

In some aspects, the present disclosure provides use of a combination of the present disclosure or a pharmaceutical composition thereof in the manufacture of a medicament for treating lung cancer.

In some aspects, the present disclosure provides use of a combination of the present disclosure or a pharmaceutical composition thereof in the manufacture of a medicament for treating or preventing non-small cell lung cancer.

In some aspects, the present disclosure provides use of a combination of the present disclosure or a pharmaceutical composition thereof in the manufacture of a medicament for treating non-small cell lung cancer.

In some embodiments, the cancer is bladder cancer, lung cancer, colon cancer, tongue cancer, sarcoma, esophageal cancer, uveal cancer, mandible cancer, larynx cancer, or mandibular gland cancer.

In some embodiments, the cancer is colorectal cancer, breast cancer, lung cancer, (e.g., non-small cell lung cancer (NSCLC)), prostate cancer, glioblastoma, mantel cell lymphoma (MCL), chronic myeloid leukemia (CML) and acute myeloid leukemia (AML), tyrosine kinase-activated leukemia, endometrial cancer, neuroblastoma, testicular cancer, germ cell tumors, Ewing's sarcoma, malignant lymphoma, ovarian cancer, fallopian tube cancer, or primary peritoneal cancer.

In some embodiments, the cancer may be selected from one or more of the group consisting of Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Cutaneous T-Cell (Mycosis Fungoides and Sezary Syndrome), Hodgkin Cancer, Non-Hodgkin Cancer, Macroglobulinemia, Waldenstrom, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenstrom Macroglobulinemia, Wilms Tumor, and Women's Cancers.

Effectiveness of combinations of the disclosure can be determined by industry-accepted assays/disease models according to standard practices of elucidating the same as described in the art and are found in the current general knowledge.

In some embodiments, the subject is fasted before the paclitaxel, Compound A, and/or the checkpoint inhibitor is administered. In some embodiments, the subject is fasted for at least 3 hours, at least 4 hours, at least 6 hours, at least 8 hours, at least 12 hours, at least 18 hours, or at least 24 hours before treatment. In some embodiments, the subject is fasted for at least 3 hours before treatment. In some embodiments, the subject is fasted for at least 4 hours before treatment. In some embodiments, the subject is fasted for at least 5 hours before treatment. In some embodiments, the subject is fasted for at least 6 hours before treatment. In some embodiments, the subject is fasted for at least 7 hours before treatment. In some embodiments, the subject is fasted for at least 8 hours before treatment. In some embodiments, the subject is fasted for at least 9 hours before treatment. In some embodiments, the subject is fasted for at least 10 hours before treatment. In some embodiments, the subject is fasted for at least 12 hours, at least 18 hours, or at least 24 hours before treatment.

In some embodiments, the subject is fasted after the paclitaxel, Compound A, and/or the checkpoint inhibitor is administered. In some embodiments, the subject is fasted for at least 30 minutes, at least 45 minutes, at least 60 minutes, at least 75 minutes, at least 90 minutes, at least 105 minutes, at least 120 minutes, at least 135 minutes, at least 150 minutes, at least 165 minutes, at least 180 minutes, at least 210 minutes, or at least 240 minutes after treatment. In some embodiments, the subject is fasted for at least 30 minutes after treatment. In some embodiments, the subject is fasted for at least 45 minutes after treatment. In some embodiments, the subject is fasted for at least 60 minutes after treatment. In some embodiments, the subject is fasted for at least 75 minutes after treatment. In some embodiments, the subject is fasted for at least 90 minutes after treatment. In some embodiments, the subject is fasted for at least 105 minutes after treatment. In some embodiments, the subject is fasted for at least 120 minutes after treatment. In some embodiments, the subject is fasted for at least 135 minutes after treatment. In some embodiments, the subject is fasted for at least 150 minutes after treatment. In some embodiments, the subject is fasted for at least 165 minutes after treatment. In some embodiments, the subject is fasted for at least 180 minutes after treatment. In some embodiments, the subject is fasted for at least 210 minutes after treatment. In some embodiments, the subject is fasted for at least 240 minutes after treatment.

In some embodiments, the size of the tumor decreases for at least 10% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 20% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 30% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 40% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 50% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 60% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 70% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 80% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 90% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor decreases for at least 95% within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, or within 20 weeks, of the administration of paclitaxel, Compound A, and the checkpoint inhibitor.

In some embodiments, the size of the tumor is measured according to RECIST v 1.1. In some embodiments, the size of the tumor is measured in at least 1 dimension and the longest diameter in the plane of measurement is recorded.

In some embodiments, there is no adverse reactions within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, within 20 weeks, within 25 weeks, within 30 weeks, within 35 weeks, or within 40 weeks, of the administration of paclitaxel. In some embodiments, there is no adverse reactions during the entire duration of the administration of paclitaxel.

In some embodiments, there is no severe adverse reactions within 1 week, within 2 weeks, within 3 weeks, within 4 weeks, within 5 weeks, within 6 weeks, within 7 weeks, within 8 weeks, within 9 weeks, within 10 weeks, within 12 weeks, within 14 weeks, within 16 weeks, within 18 weeks, within 20 weeks, within 25 weeks, within 30 weeks, within 35 weeks, or within 40 weeks, of the administration of paclitaxel. In some embodiments, there is no severe adverse reactions during the entire duration of the administration of paclitaxel.

An adverse reaction, as used herein, is any reaction caused by or associated with the administration of the paclitaxel, Compound A, and/or the checkpoint inhibitor that is or is considered to be undesirable or harmful. An adverse reaction can be mild, moderate, or severe, depending on the type of the adverse reaction and/or the level/amount of the adverse reaction. In some embodiments, the adverse reaction is gastrointestinal (e.g., vomiting, nausea, abdominal discomfort, or diarrhea). In some embodiments, the adverse reaction is neurotoxicity (e.g., peripheral neuropathy). In some embodiments, the adverse reaction is hematological in nature (e.g., hematological toxicities associated with IV paclitaxel). In some embodiments, the adverse reaction is leucopenia. In some embodiments, the adverse reaction is febrile neutropenia. In some embodiments, the adverse reaction is neutropenia. In some embodiments, the adverse reaction is neutropenia with infection. In some embodiments, the adverse reaction is neutropenia without infection. In some embodiments, the adverse reaction is any unfavorable or unintended sign. In some embodiments, the adverse reaction is any new disease or exacerbation of an existing disease. In some embodiments, the adverse reaction is any deterioration in nonprotocol-required measurements of a laboratory value or other clinical test. In some embodiments, the adverse reaction is recurrence of an intermittent medical condition. In some embodiments, the adverse reaction is headache. In some embodiments, the adverse reaction is life threatening.

In some embodiments, an adverse reaction includes neutropenia, alopecia, anemia, arthralgia/myalgia, diarrhea, leukopenia, nausea/vomiting, opportunistic infections, peripheral neuropathy, thrombocytopenia, mucositis, hypersensitivity, renal impairment, hypotension, bradycardia, grand mal seizures, cardiac conduction abnormalities, Pyrexia, dehydration, pancytopenia, congestive heart failure, left ventricular dysfunction, Stevens-Johnson syndrome, toxic epidermal necrolysis, and extravasation.

In some embodiments, the subject meets the Inclusion Criteria for treatment. In some embodiments, the subject is 18 years or older. In some embodiments, the subject has a tumor that was not amenable to prior therapies (e.g., a monotherapy of nivoluman or pembrolizumab). In some embodiments, the prior therapy is selected from, but not limited to, interleukin, nivolumab, gemcitabine, pemetrexed, radiation, cisplatin, trametinib, atezolizumab, bevacizumab, carboplatin, lobectomy, lymph node dissection, whole brain radiation, erlotinib, docetaxel/bevacizumab, and monotherapy pembrolizumab. In some embodiments, the cancer is relapsed. In some embodiments, the cancer is refractory. In some embodiments, the disease is measurable per RECIST v.1.1. In some embodiments, the subject has an Eastern Cooperative Oncology Group (ECOG11) performance status ≤1. In some embodiments, the subject has resolution of all acute AEs from prior cancer therapies to NCI CTCAE v4.03 Grade ≤1 or to that subject's baseline.

In some embodiments, the subject has adequate organ function. In some embodiments, adequate organ function is renal function as evidenced by serum creatinine ≤1.5×upper limit of normal (ULN) or calculated creatinine clearance ≥50 mL/min per the Cockcroft and Gault formula. In some embodiments, adequate organ function is bone marrow function as evidenced by absolute neutrophil count, hemoglobin, and platelet count. In some embodiments, adequate organ function is liver function as evidenced by total bilirubin within normal limits, alanine aminotransferase (ALT)≤3×ULN, and aspartate aminotransferase (AST)≤3×ULN, gamma-glutamyl transferase (GGT)≤10×ULN, and alkaline phosphatase ≤3×ULN.

In some embodiments, the subject is male. In some embodiments, the subject is male and sterile (including vasectomy confirmed by post vasectomy semen analysis).

In some embodiments, the subject is female. In some embodiments, the female is of non-child bearing potential due to surgical sterilization confirmed by medical history or menopause.

In some embodiments, the female is of child-bearing potential and tests negative for pregnancy based on serum pregnancy test.

In some embodiments, the subject has a life expectancy of at least 3 months.

In some embodiments, the subject is between the ages of about 0 years old to about 110 years old. In some embodiments, the subject is between the ages of about 0 years old to about 10 years old. In some embodiments, the subject is between the ages of about 10 years old to about 20 years old. In some embodiments, the subject is between the ages of about 20 years old to about 30 years old. In some embodiments, the subject is between the ages of about 30 years old to about 40 years old. In some embodiments, the subject is between the ages of about 40 years old to about 50 years old. In some embodiments, the subject is between the ages of about 50 years old to about 60 years old. In some embodiments, the subject is between the ages of about 60 years old to about 70 years old. In some embodiments, the subject is between the ages of about 70 years old to about 80 years old. In some embodiments, the subject is between the ages of about 80 years old to about 90 years old. In some embodiments, the subject is between the ages of about 90 years old to about 100 years old. In some embodiments, the subject is between the ages of about 90 years old to about 110 years old. In some embodiments, the subject is between the ages of about 60 years old to about 110 years old. In some embodiments, the subject is between the ages of about 60 years old to about 100 years old. In some embodiments, the subject is between the ages of about 60 years old to about 90 years old. In some embodiments, the subject is between the ages of about 60 years old to about 80 years old. In some embodiments, the subject is between the ages of about 60 years old to about 70 years old.

Unless explicitly indicated otherwise, the terms “approximately” and “about” are synonymous. In some embodiments, “approximately” and “about” refer to the recited amount, dose, value (for example, AUC_{(0→∞)}, or duration ±20%, ±15%, ±10%, ±8%, ±6%, ±5%, ±4%, ±2%, ±1%, or ±0.5%. In another embodiment, “approximately” and “about” refer to the listed amount, dose, value, or duration ±10%, ±8%, ±6%, ±5%, ±4%, or ±2%. In yet another embodiment, “approximately” and “about” refer to the listed amount, dose, value, or duration ±5%. In yet another embodiment, “approximately” and “about” refer to the listed amount, dose, value, or duration ±2%. In yet another embodiment, “approximately” and “about” refer to the listed amount, dose, value, or duration ±1%. When the terms “approximately” and “about” are used when reciting temperature or temperature range, these terms refer to the recited temperature or temperature range±5° C., ±2° C., or ±1° C. In another embodiment, the terms “approximately” and “about” refer to the recited temperature or temperature range±2° C.

The term “paclitaxel” refers to 5,20-Epoxy-1,2,4,7,10-13-hexahydroxytax-11-en-9-one-4,10-diacetate 2-benzoate 13-ester with (2R,3S)-N-benzoyl-3-phenylisoserine, i.e., the compound with the following structure:

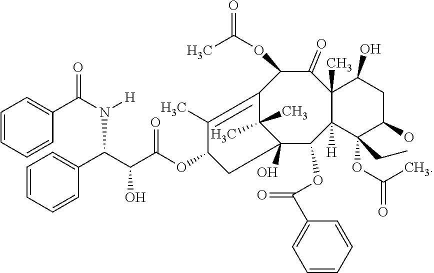

Unless otherwise indicated, the term “paclitaxel” includes pharmaceutically acceptable salts and/or solvates thereof.

Paclitaxel suitable for intravenous administration or intravenously administered paclitaxel includes compositions that comprise paclitaxel and a pharmaceutical excipient or carrier that facilitates the intravenous administration of paclitaxel. Such pharmaceutical excipient or carrier includes polyethoxylated castor oil, e.g., Cremophor®. In some embodiments, paclitaxel suitable for intravenous administration or intravenously administered paclitaxel includes the brand name product, TAXOL®, and generic versions thereof. In some embodiments, paclitaxel suitable for intravenous administration or intravenously administered paclitaxel includes a composition comprising protein-bound paclitaxel, i.e., Abraxane®. In some embodiments, paclitaxel suitable for intravenous administration exhibits clinical toxicities. In some embodiments, paclitaxel suitable for intravenous administration exhibits clinical toxicities due to the excipient or carrier. In some embodiments, paclitaxel suitable for intravenous administration exhibits clinical toxicities due to the polyethoxylated castor oil excipient or carrier.

Paclitaxel suitable for oral administration or orally administered paclitaxel refers to a formulation of paclitaxel that is administered orally. In some embodiments, the orally administered paclitaxel is in capsule form. In some embodiments, each capsule contains paclitaxel and a surfactant, e.g., polysorbate 80. In some embodiments, each capsule contains 30 mg-200 mg of paclitaxel. In some embodiments, each capsule contains 30 mg, 45 mg, 60 mg, 75 mg, 90 mg, 100 mg, 125 mg, 150 mg, 175 mg, or 200 mg of paclitaxel. In some embodiments, each capsule contains 30 mg of paclitaxel. In some embodiments, each capsule contains 500 mg of polysorbate 80. In some embodiments each capsule contains 30 mg of paclitaxel and 500 mg of polysorbate 80. In some embodiments, the orally administered paclitaxel is in tablet form. In some embodiments, each tablet contains 30 mg-200 mg of paclitaxel. In some embodiments, each tablet contains 30 mg, 45 mg, 60 mg, 75 mg, 90 mg, 100 mg, 125 mg, 150 mg, 175 mg, or 200 mg of paclitaxel. In some embodiments, each tablet contains 30 mg of paclitaxel.

Oral formulation of paclitaxel (e.g., capsule or tablet) may be formulated by any suitable methods known in the art.

The term “Compound A” refers to a compound, or a pharmaceutically acceptable salt and/or solvate thereof, which is a P-gp pump inhibitor and has the following structure:

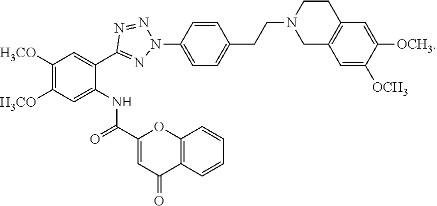 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]

Unless indicated otherwise, the terms “Compound A,” “HM30181 methanesulfonate monohydrate,” “HM30181A,” “HM30181 AK,” and “HM30181 AK-US” are all equivalent and are used interchangeably. In some embodiments, Compound A refers to a methanesulfonate salt monohydrate of Compound A:

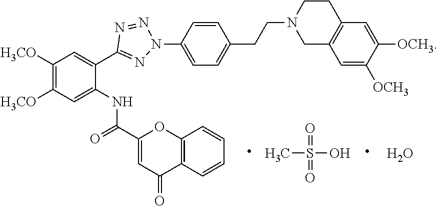

In some embodiments, Compound A refers to a methanesulfonate salt monohydrate of Compound A. Compound A is commercially available, e.g., in tablet form suitable for oral administration. In some embodiments, Compound A is administered in 15 mg tablets suitable for oral administration.

Compound A may be formulated by any suitable methods known in the art.

The term “checkpoint inhibitor” refers to a compound selected from, but not limited to, anti-PD-1 antibody, an anti-PD-L1 antibody, an anti-CTLA4 antibody, an anti-A2AR antibody, an anti-B7-H3 antibody, an anti-B7-H4 antibody, an anti-BTLA antibody, an anti-ICOS antibody, an anti-OX-40 antibody, an anti-41BB antibody, an anti-BITR antibody, an anti-IDO antibody, an anti-KIR antibody, an anti-LAG3 antibody, an anti-TIM3 antibody, and an anti-VISTA (V-domain Ig suppressor of T cell activation) antibody.

In some embodiments, the checkpoint inhibitor is a small-molecule compound or peptidic molecule selected from, but not limited to, small-molecule inhibitors of PD-1 and PDl-1 (e.g., PD-1/PD-L1 Inhibitor 3, BMS202, AUNP-12, and PD-1/PD-L1 inhibitor 1).

The term “pembrolizumab” refers to a compound, or a pharmaceutically acceptable salt and/or solvate thereof, which is a checkpoint inhibitor. Unless indicated otherwise, the terms “pembrolizumab,” “pem,” “Keytruda,” “ML-3475,” and “lambrolizumab” are all equivalent and are used interchangeably.

Pembrolizumab may be formulated by any suitable methods known in the art.

The term “subject” includes any living organism that has an angiosarcoma or is at a risk of developing an angiosarcoma. In some embodiments, the term “subject” refers to a mammal that has an angiosarcoma or is at a risk of developing an angiosarcoma. In some embodiments, the term subject refers to a human being that has an angiosarcoma or is at a risk of developing an angiosarcoma. In some embodiments, the term subject refers to an angiosarcoma patient, i.e., a patient.

The term “AUC_{(o→∞)}” refers to the total exposure to a drug and is expressed in unit of concentration time. In some embodiments, it is the concentration of a drug over a time interval circulating in the body, e.g., in the plasma, blood, or serum. In some embodiments, AUC_{(o→∞)}=AUC_{(o→Tlast)}+(C_{Tlast}/K_{clim}), where C_{Tlast} is the last measurable drug concentration and Kₑₗᵢₘ is the terminal elimination rate constant, expressed in time⁻¹ units.

In some embodiments, C_{Tlast} may be determined from about 1 day to about 21 days after oral administration of paclitaxel.

In some embodiments, C_{Tlast} may be determined from about 2 days to about 14 days after oral administration of paclitaxel.

In some embodiments, C_{Tlast} may be determined from about 3 days to about 7 days after oral administration of paclitaxel.

In some embodiments, C_{Tlast} may be determined at about 3 days after oral administration of paclitaxel.

In some embodiments, C_{Tlast} may be determined at about 4 days after oral administration of paclitaxel.

In some embodiments, C_{Tlast} may be determined at about 5 days after oral administration of paclitaxel.

In some embodiments, C_{Tlast} may be determined at about 6 days after oral administration of paclitaxel.

In some embodiments, C_{Tlast} may be determined at about 7 days after oral administration of paclitaxel.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject depends upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any compound, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active ingredient or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing paclitaxel may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active ingredient into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF, Parsippany, N.J.) or phosphate buffered saline (PBS). In all cases, the composition is sterile and should be fluid to the extent that easy syringeability exists. The composition is stable under the conditions of manufacture and storage and is preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating paclitaxel in the required amount in an appropriate solvent with one or a combination of ingredients enumerated above, as required, followed by filtered sterilization. Generally, dispersions are prepared by incorporating form paclitaxel into a sterile vehicle that contains a basic dispersion medium and the required other ingredients from those enumerated above. In the case of sterile powders for the preparation of sterile injectable solutions, methods of preparation are vacuum drying and freeze-drying that yields a powder of paclitaxel plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, form can be incorporated with excipients and used in the form of tablets, troches, or capsules. Oral compositions can also be prepared using a fluid carrier for use as a mouthwash, wherein in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The pharmaceutical compositions described herein can be included in a container, pack, or dispenser together with instructions for administration.

All percentages and ratios used herein, unless otherwise indicated, are by weight.

Other features and advantages of the present application are apparent from the different examples. The provided examples illustrate different components and methodology useful in practicing the present application.

The examples do not limit the claimed application. Based on the present disclosure the skilled artisan can identify and employ other components and methodology useful for practicing the present application.

### Examples

### Example 1—Safety and Tolerability of Oral Paclitarel, Compound A, and Pembrolizumab

A clinical study was conducted to determine the safety and tolerability and recommended phase 2 dose (RP2D) and to evaluate early clinical activity. The dose escalation utilized the “3+3” design (Table 1). Eligible patients had metastatic or unresectable solid tumors for which Pem is an FDA approved therapy. The oral paclitaxel dose range explored was 270-330 mg administered for 2 days up to a maximum of 5 days per week×2 weeks of a 3-week cycle. Pembrolizumab 200 mg IV was administered every three weeks. PK samples were collected for PK-PD analysis.

In total, 21 patients, age range 45 to 81 years were enrolled, including 10 lung cancer, 4 head and neck cancer (H&N), 3 oesophageal cancer, 1 bladder cancer, 1 colon cancer, 1 sarcoma, and 1 uveal cancer. The MTD was not reached. 1 DLT (febrile neutropenia grade (G) 4) was observed at the 270 mg×4 days dose level. 3 patients experienced neutropaenia (2 patients G3, 1 patient G4). Three patients experienced SAEs related to treatment (neutropenia G3 and G4 and diarrhea G3). No deaths due to treatment occurred. Clinical activity was seen at all dose levels. Of 15 evaluable patients 6 had a partial response (5 lung cancer, 1 colon cancer), 6 had stable disease (2 lung cancer, 2 H&N, 1 uveal cancer, 1 oesophageal), and 3 had progressive disease (1 H&N, 1 bladder, 1 oesophageal). PK analysis of oral paclitaxel identified an approximate linear relationship between dose and AUCt and no difference from historic monotherapy PK. The study determined 270 mg×3 days per week for 2 weeks (Days 1-3, 8-10) of a 3-week cycle as the RP2D.

The study demonstrated that addition of oral paclitaxel and Encequidar to pembrolizumab was safe, and an RP2D was identified.

### Subject Eligibility

Histologically confirmed metastatic or unresectable solid tumours for which pembrolizumab is an FDA-approved therapy.
Previously professed on or become intolerant of at least one line of systemic chemotherapy for metastatic or advanced disease
Adequate baseline hepatic, hematological and renal function.
At least one measurable site of disease as defined as per RECIST v1.1 criteria

### Objectives

The primary objectives for Part A of the study, was to determine the maximum tolerated dose (MTD) and identify the recommended Phase 2 dose (RP2D) of oral paclitaxel and Encequidar in combination with pembrolizumab in subjects with advanced solid tumours.
Secondary objectives included safety and tolerability, the PK of paclitaxel and preliminary anti-tumour activity.

### Study Design

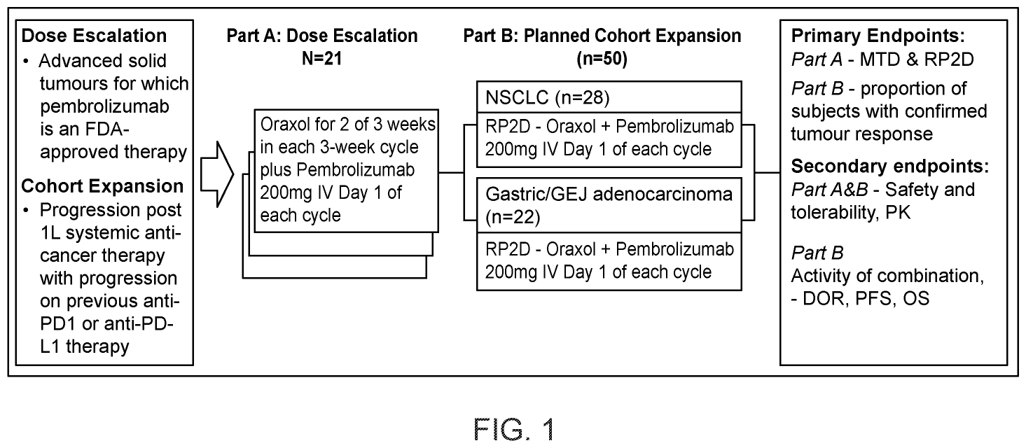

Open-label, Phase 1 dose-escalation “3+3” design study followed by a 2-arm expansion cohort (FIG. 1).
MTD and dose limiting toxicities (DLT) were determined based on toxicities observed during the first 3-week cycle of treatment.
Response was determined by CT scan evaluated by RECIST 1.1 every 9 weeks.
The Part B dose expansion phase will enroll subjects into 2 independent cohorts to further evaluate the activity, safety and tolerability of the study treatment in subjects with advanced/metastatic gastric/gastro-esophageal cancer or non-small cell lung cancer.

TABLE 1
Dosage Regimen

| | | | Oral Paclitaxel | | |
|---|---|---|---|---|---|
| | Compound | Oral | and Compound | Pembrolizumab | Subjects |
| Cohort | A | Paclitaxel | A Dosing Days | (IV) | Enrolled |
| 1 | 15 mg | 270 mg | Days 1-2 for 2 of | 200 mg Q3W | 3 |
| | | | 3 weeks | | |
| 2 | 15 mg | 270 mg | Days 1-3 for 2 of | 200 mg Q3W | 3 |
| | | | 3 weeks | | |
| 3 | 15 mg | 270 mg | Days 1-4 for 2 of | 200 mg Q3W | 6 |
| | | | 3 weeks | | |
| 4 | 15 mg | 270 mg | Days 1-5 for 2 of | 200 mg Q3W | 3 |
| | | | 3 weeks | | |
| 5 | 15 mg | 300 mg | Days 1-5 for 2 of | 200 mg Q3W | 3 |
| | | | 3 weeks | | |
| 6 | 15 mg | 330 mg | Days 1-5 for 2 of | 200 mg Q3W | 3 |
| | | | 3 weeks | | |

### Subject Characteristics

Subject characteristics are presented in Table 2.
21 Subjects with a mean age of 65.6 years were enrolled into Part 1, including 10 subjects with non-small cell lung cancer.
PD-L1 status: six positive, four negative, and 11 unknown.
In the subject of 10 subjects with non-small cell lung cancer:
   PD-L1 status was available in seven subjects: three were PD-L1 positive, four were PD-L1 negative
   Five subjects had received prior pembrolizumab, three received prior nivolumab, one received prior atezolizumab, and one received prior nivolumab and atezolizumab.

TABLE 2
Subject Characteristics

| | Oral Paclitaxel, Compound A, |
|---|---|
| | Pembrolizumab (N = 21) |
| Age (Years) | |
| Mean (SD) | 65.5 (9.16) |
| Median | 65 |
| Race, n (%) | |
| Black or African American | 1 (5) |
| White | 20 (95) |
| Baseline ECOG, n (%) | |
| 0 | 7 (33) |
| 1 | 14 (67) |
| Tumour type | |
| Non-small cell lung cancer | 10 (48) |
| Head and neck | 4 (19) |
| Oesphageal cancer | 3 (14) |
| Bladder cancer | 1 (5) |
| Colon cancer | 1 (5) |
| Sarcoma | 1 (5) |
| Uveal melanoma | 1 (5) |
| PD-L1 status, n (%) | |
| Positive | 6 (29) |
| Negative | 4 (19) |
| Not Available | 11 (52) |
| Prior taxane exposure, n (%) | |
| Docetaxel/paclitaxel | 4 (20) |
| Subjects with visceral metastases, | 13 (62) |
| n (%) | |
| Prior chemotherapy regimens, n (%) | |
| No prior chemotherapy | 0 |
| 1 Line | 3 (14) |
| ≥2 lines | 18 (86) |

The dose range (270, 300, and 330 mg) resulted in a linear PK. PK profiles and derived paclitaxel parameters after administration or oral paclitaxel [Cₘₐₓ 161 ng/mL; (CV % 62); AUC₀₋₂₄ 692 h*ng/mL. (CV % 64)] in combination with pembrolizumab were similar to those previously reported for oral paclitaxel monotherapy. The MTD was not reached at the highest dose level of oral paclitaxel (330 mg QD on Days 1-5/week for 2 weeks of each 3-week cycle) and Compound A at 15 mg. A summary of the grade≥3 toxicities are presented in Table 3 (Compound A was administered at 15 mg). One dose-limiting toxicity was observed: febrile neutropenia occurred in cohort 3. Two additional subjects experienced febrile neutropenia: one in cohort five during cycle 2 and one in cohort six during cycle 5. No deaths were attributed to the treatment of oral paclitaxel and Compound A. One fatal pneumonitis occurred in cohort six during cycle 8 and was attributed to pembroliztunab. The RP2D of oral paclitaxel and Compound A in combination with pembrolizumab was determined.

TABLE 3
Safety Summary

| | Oral | | |
|---|---|---|---|
| | Paclitaxel | Treatment-Related | |
| Cohort | Dose | Toxicity (Grade ≥3) | Cycle AE Occurred |
| 1 | 270 mg, | Anemia (G3) | 5 |
| | Days 1-2 | | |
| 2 | 270 mg, | Maculopapular Rash (G3) | 3 |
| | Days 1-3 | | |
| 3 | 270 mg, | Alt Increased (G3) | 3 |
| | Days 1-4 | Bilirubin Increased (G4) | 3 |
| | | Anemia (G3) | 2 |
| | | Anorexia (G3) | 1 |
| | | Febrile Neutropenia (G3)* | 1 |
| | | Pneumonia (G3) | 1 |
| 4 | 270 mg, | Neutropenia (G4) | 1 |
| | Days 1-5 | Diarrhea (G3) | 2 |
| 5 | 300 mg, | Febrile Neutropenia (G3) | 2 |
| | Days 1-5 | | |
| 6 | 330 mg, | GGT Elevation (G3) | 1 |
| | Days 1-5 | Pneumonitis (G5) | 9 |
| | | Febrile Neutropenia (G4) | 5 |
| | | Sepsis (G4) | 5 |
| | | Neutropenia (G3) | 1 |

*Indicates dose limiting toxicity

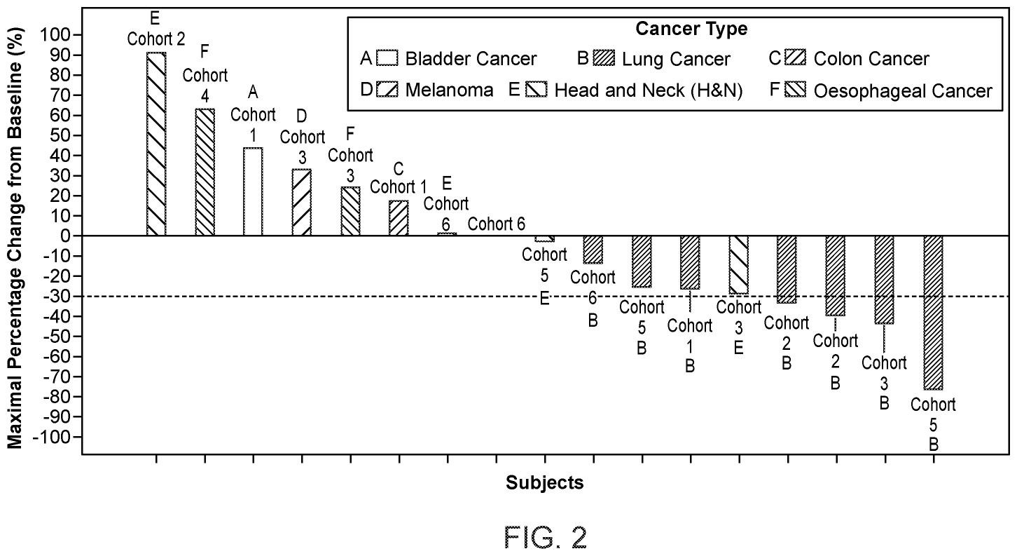

Overall, 17 subjects were evaluable for a response: 4 had partial response (4 non-small cell lung cancer), 10 had stable disease (4, non-small cell lung cancer, 3 head and neck cancer, 1 uveal melanoma, 1 oesophageal cancer, and 1 colon cancer), 3 had progressive disease (1 head and neck, 1 bladder cancer, 1 oesophageal cancer) (FIG. 2). Duration on treatment ranged from 9 to over 676 days.

Non-Small Cell Lung Cancer (n=10)

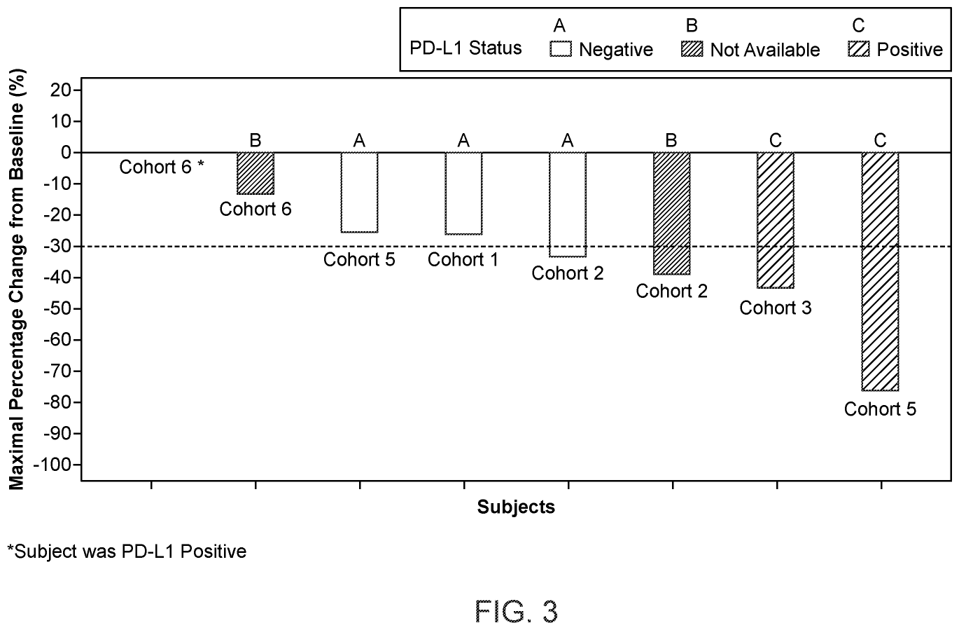

There were eight evaluable non-small cell lung cancer subjects of which four had PR and four subjects had SD, the overall response rate (ORR) was 40% (see FIG. 3). Ten subjects had prior anti-PD-1/anto-PD-L1 therapy:
Five subjects had prior pembrolizumab. All discontinued due to progressive disease. Best responses were—2 PRs, 2 SDs, 1 response (non-RECIST).
Three subjects last prior immunotherapy was nivolumab. All discontinues due to progressive disease. Best prior responses were—1 PR and 2 PDs.
Two subjects last prior immunotherapy was atezolizumab. All discontinued due to progressive disease. Best prior responses were—1 SD, 1 response (non-RECIST).
Only one subject had prior taxane therapy.

In the current study, of the three subjects who were PD-L1 positive, two subjects had a PR and 1 had a SD; of the four PD-L1 negative subjects, one had PR, two had SD and one was not evaluable (FIG. 3).

### Results

The combination of oral paclitaxel and Compound A and pembrolizumab had a tolerable safety profile. The MTD of the combination was not reached. The RP2D in combination with pembrolizumab was identified as oral paclitaxel 270 mg QD Days 1-3 for 2 weeks of a 3-week cycle and 15 mg Compound A. Anti-tumor activity was seen in subjects with non-small cell lung cancer who had previously progressed on prior checkpoint inhibitor therapy, with an ORR of 40%. Part B expansion cohorts are enrolling subjects with non-small cell lung cancer or gastric/GEJ carcinoma to further evaluate the safety and promising clinical activity of oral paclitaxel and Compound A with pembrolizumab.

### INCORPORATION BY REFERENCE

The entire disclosure of each of the patent documents and scientific articles referred to herein is incorporated by reference for all purposes.

### EQUIVALENTS

The disclosure of the application can be embodied in other specific forms without departing from the spirit or essential characteristics thereof. The foregoing embodiments are therefore to be considered in all respects illustrative rather than limiting on the disclosure of the application described herein. Scope of the disclosure of the application is thus indicated by the appended claims rather than by the foregoing description, and all changes that come within the meaning and range of equivalency of the claims are intended to be embraced therein.

What is claimed is:

1. A method for treating a cancer in a subject in need thereof, comprising:
a. oral administration of paclitaxel at an amount of about 270 mg to the subject once a day and for 3 times a week; and
b. oral administration of Compound A: 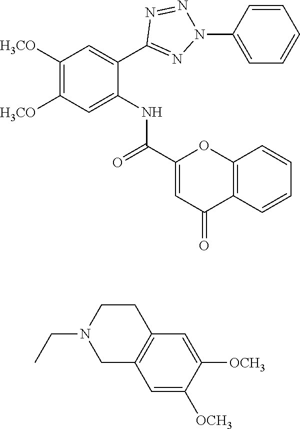 [SMILES: COc1cc(NC(=O)c2cc(=O)c3ccccc3o2)c(-c2nnn(-c3ccccc3)n2)cc1OC ; CCN1CCc2cc(OC)c(OC)cc2C1]
to the subject once a day and for 1-3 times a week; and
c. intravenous administration of a checkpoint inhibitor, wherein Compound A is administered prior to the paclitaxel.

2. The method of claim 1, comprising oral administration of paclitaxel on consecutive days.

3. The method of claim 1, comprising oral administration of Compound A at an amount of about 15 mg.

4. The method of claim 1 or 2, comprising oral administration of Compound A for 3 times a week.

5. The method of claim 1, comprising oral administration of Compound A on consecutive days.

6. The method of claim 1, comprising oral administration of Compound A at an amount of about 15 mg and for 3 times a week.

7. The method of claim 1, wherein the check point inhibitor is pembrolizumab.

8. The method of claim 1, comprising intravenous administration of the checkpoint inhibitor at an amount of about 200 mg or 400 mg.

9. The method of claim 1, wherein the checkpoint inhibitor is administered one every three weeks or six weeks.

10. The method of claim 1, wherein the cancer is lung cancer.

11. The method of claim 10, wherein the lung cancer is non-small cell lung cancer.

12. A method for treating a cancer in a subject in need thereof, comprising:
a. oral administration of paclitaxel at an amount of about 300 mg to the subject once a day and for 3 times a week; and
b. oral administration of Compound A: 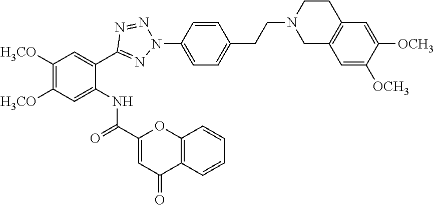 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]
to the subject once a day and for 1-3 times a week; and
c. intravenous administration of a checkpoint inhibitor, wherein Compound A is administered prior to the paclitaxel.

13. A method for treating a cancer in a subject in need thereof comprising:
a. oral administration of paclitaxel at an amount of about 330 mg to the subject once a day and for 3 times a week; and
b. oral administration of Compound A: 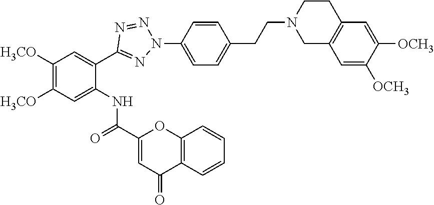 [SMILES: COc1cc2c(cc1OC)CN(CCc1ccc(-n3nnc(-c4cc(OC)c(OC)cc4NC(=O)c4cc(=O)c5ccccc5o4)n3)cc1)CC2]
to the subject once a day and for 1-3 times a week; and
c. intravenous administration of a checkpoint inhibitor, wherein Compound A is administered prior to the paclitaxel.
